(19) United States
(12) Patent Application Publication
(10) Pub. No.: US 2021/0299237 A1
(43) Pub. Date: Sep. 30, 2021

(54) TRITERPENE SAPONIN SYNTHESIS, INTERMEDIATES AND ADJUVANT COMBINATIONS

(71) Applicant: ADJUVANCE TECHNOLOGIES, INC., New York, NY (US)

(72) Inventors: Amanda CHAN, Hopewell Junction, NY (US); Jeffrey GARDNER, New York, NY (US); Lars NORDSTROEM, New York, NY (US); William WALKOWICZ, Brooklyn, NY (US); J Tyler MARTIN, Roca, NE (US); Weidong Zhang, Albany, NY (US)

(73) Assignee: ADJUVANCE TECHNOLOGIES, INC., New York, NY (US)

(21) Appl. No.: 16/604,867

(22) PCT Filed: Apr. 13, 2018
(86) PCT No.: PCT/US2018/027462

§ 371 Date: Oct. 11, 2019

Related U.S. Application Data
(60) Provisional application No. 62/485,260, filed on Apr. 13, 2017.
(60) Provisional application No. 62/488,287, filed on Apr. 21, 2017.
(60) Provisional application No. 62/489,546, filed on Apr. 25, 2017.

(51) Int. Cl. A61K 39/00; C07H 1/08; C07H 15/18; A61K 39/05; A61K 39/08; A61K 39/09; A61K 39/02; A61K 39/29; A61K 39/39; A61P 31/20; A61P 31/04

(52) U.S. Cl. CPC A61K 39/0018; C07H 1/08; C07H 15/18; A61K 39/05; A61K 39/08; A61K 39/092; A61K 2039/55577; A61K 39/292; A61K 39/39; A61P 31/20; A61P 31/04; A61K 2039/6037; A61K 39/099

(57) ABSTRACT

The present application relates to triterpene glycoside saponin-derived adjuvants, syntheses thereof, and intermediates thereto. The application also provides pharmaceutical compositions comprising compounds of the present invention and methods of using said compounds or compositions in the treatment of and immunization for infectious diseases.

### INCORPORATION BY REFERENCE OF RELATED PATENT APPLICATIONS

This application is based upon and claims priority under 35 U.S.C. § 119(e) to U.S. provisional application U.S. Ser. No. 62/485,260 filed Apr. 13, 2017, to U.S. provisional application U.S. Ser. No. 62/488,287 filed Apr. 21, 2017, and to U.S. provisional application U.S. Ser. No. 62/489,546 filed Apr. 25, 2017, the entire contents of which are incorporated herein by reference in their entirety.

### GOVERNMENT SUPPORT

Some embodiments of the subject matter in this application were made with United States Government support under grant GRANT11540722 awarded by the National Institutes of Health. The United States Government has certain rights in the subject matter of this application.

### FIELD OF THE INVENTION

The present application relates to triterpene glycoside saponin-derived adjuvants, syntheses thereof, and intermediates thereto. The application also provides pharmaceutical compositions comprising compounds of the present invention and methods of using said compounds or compositions in the treatment of infectious diseases.

### BACKGROUND

Vaccines against infectious diseases continue to improve public health across the world. With increased knowledge of etiologic pathogens and necessary immune responses have come increasingly defined or targeted vaccines. Hepatitis B, DTaP, HPV, pneumococcal and other widely used vaccines require use of the immunological adjuvant alum. However, alum, which was introduced over 80 years ago, is a poor adjuvant restricting the potency of some of these vaccines and requiring higher or more doses of others. A leading candidate as a far more potent adjuvant than alum is the natural saponin adjuvant QS-21, used widely despite 3 major liabilities: dose limiting toxicity, poor stability, and limited availability of quality product.

Saponins are glycosidic compounds that are produced as secondary metabolites of steroids and triterpenes. The chemical structure of saponins imparts a wide range of pharmacological and biological activities, including some potent and efficacious immunological activity. Semi-purified saponin extracts from the bark of the South American Quillaja saponaria Molina tree (Quillaja saponins) exhibit remarkable immunoadjuvant activity. Because the Quillaja saponins are found as a mixture of at least one hundred structurally related saponin glycosides, their separation and isolation is often difficult if not prohibitive. The most active fraction of these extracts, designated QS-21, has been found to include a mixture of two principal isomeric triterpene glycoside saponins, each incorporating a quillaic acid triterpene core, flanked on either side by complex oligosaccharides and a stereochemically rich glycosylated fatty acyl chain.

The potency of QS-21 and its favorable toxicity profile in dozens of recent and ongoing vaccine clinical trials (melanoma, breast cancer, small cell lung cancer, prostate cancer, HIV-1, malaria) have established it as a promising new adjuvant for immune response potentiation and dose-sparing. However, the tolerated dose of QS-21 in cancer patients does not exceed 100-150 μg, above which significant local and systemic side effects arise. The highest practical tolerable dose in well (non-cancer) adult and child recipients is 25-50 mcg, an immunologically suboptimal dose. As a result, the clinical success of non-cancer vaccines continues to critically depend on the identification of, and access to, novel, potent adjuvants that are more tolerable.

### SUMMARY

The present invention encompasses the recognition that the clinical use of QS-21 as an adjuvant is limited due to toxicity at higher doses, and that QS-7, a related Quillaja saponin, is difficult to isolate in pure form. Moreover, synthetic access to QS-21, QS-7, and other triterpene glycoside saponins is hindered by their structural complexity. The present application provides compounds that are analogs of QS-21 and QS-7.

In one aspect, the present application provides compounds of Formula I:

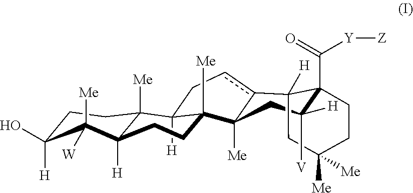

or a pharmaceutically acceptable salt thereof, wherein
[symbol] is a single or double bond;
W is —CHO;
V is hydrogen or OR^{x};
Y is CH₂, —O—, —NR—, or —NH—;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

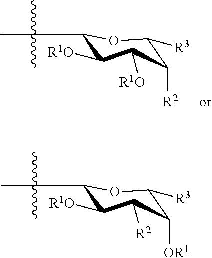

wherein each occurrence of R¹ is R^{x} or a carbohydrate domain having the structure:

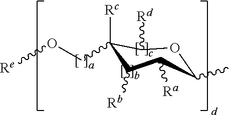

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

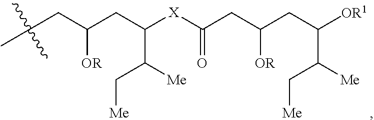

wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, carbamates, and carbonates;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
   two R on the same nitrogen atom are taken with the nitrogen atom to form a 4-7-membered heterocyclic ring having 1-2 heteroatoms independently selected from the group consisting of nitrogen, oxygen, and sulfur.

In one aspect, the present application provides compounds of Formula II:

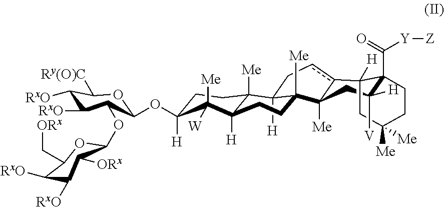

or a pharmaceutically acceptable salt thereof, wherein
[symbol] is a single or double bond;
W is Me, —CHO, or

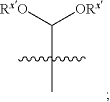

V is hydrogen or OR^{x};
Y is CH₂, —O—, —NR—, or —NH—;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

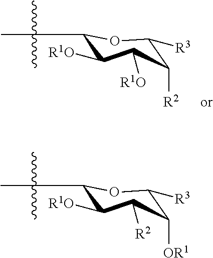

wherein each occurrence of R¹ is R^{x} or a carbohydrate domain having the structure:

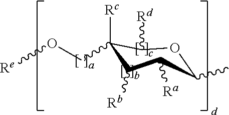

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;

R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

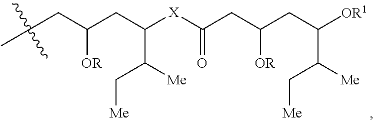

wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, carbamates, and carbonates;
R^{y} is —OH, —OR, or a carboxyl protecting group selected from the group consisting of ester, amides, and hydrazides;
R^{s} is

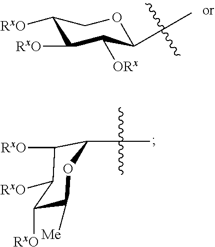

each occurrence of R^{x′} is independently an optionally substituted group selected from 6-10-membered aryl, C₁₋₆ aliphatic, or C₁₋₆ heteroaliphatic having 1-2 heteroatoms independently selected from the group consisting of nitrogen, oxygen, and sulfur; or:
   two R^{x′} are taken together to form a 5-7-membered heterocyclic ring having 1-2 heteroatoms independently selected from the group consisting of nitrogen, oxygen, and sulfur;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
   two R on the same nitrogen atom are taken with the nitrogen atom to form a 4-7-membered heterocyclic ring having 1-2 heteroatoms independently selected from the group consisting of nitrogen, oxygen, and sulfur.

It will be appreciated by one of ordinary skill in the art that the compounds of the present application include, but are not necessarily limited to, those compounds encompassed in the genus set forth herein. The compounds encompassed by this application include at least all of the compounds disclosed in the entire specification as a whole, including all individual species within each genus.

In another aspect, the present invention provides novel semi-synthetic methods for synthesizing QS-7, QS-21, and related analogs, the method comprising coupling a triterpene compound with a compound comprising a saccharide to form a compound of Formula II. In some embodiments, the method comprises the steps of:
(a) providing a compound of Formula III:

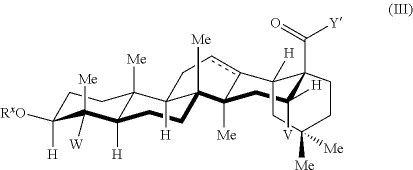

wherein:
[symbol] is a single or double bond;
Y′ is hydrogen, halogen, alkyl, aryl, OR, OR^{y}, OH, NR₂, NR₃⁺, NHR, NH₂, SR, or NROR;
W is Me, —CHO, —CH₂OR^{x}, —C(O)R^{y}, or

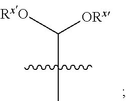

V is hydrogen or —OR^{x};
R^{y} is —OH, or a carboxyl protecting group selected from the group consisting of ester, amides, and hydrazides;
each occurrence of R^{x′} is independently an optionally substituted group selected from 6-10-membered aryl, C1-6 aliphatic, or C₁₋₆heteroaliphatic having 1-2 heteroatoms independently selected from the group consisting of nitrogen, oxygen, and sulfur; or:
   two R^{x′} are taken together to form a 5-7-membered heterocyclic ring having 1-2 heteroatoms independently selected from the group consisting of nitrogen, oxygen, and sulfur;
each occurrence of R is independently hydrogen, an optionally substituted group selected from acyl, arylalkyl, 6-10-membered aryl, C₁₋₁₂ aliphatic, or C₁₋₁₂ heteroaliphatic having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, and carbonates;
(b) treating said compound of Formula III under suitable conditions with a compound of Formula V:

LG-Z (V)
wherein:
Z is hydrogen; a cyclic or acyclic, optionally substituted moiety selected from the group consisting of acyl, aliphatic, heteroaliphatic, aryl, arylalkyl, and heteroaryl; or a carbohydrate domain having the structure:

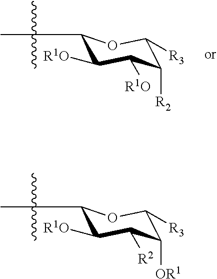

wherein:
each occurrence of R¹ is Rx or a carbohydrate domain having the structure:

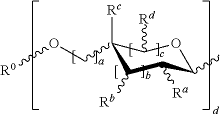

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

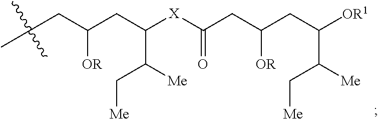

wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR^{1′}, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
R^{1′} is R^{x} or a carbohydrate domain having the structure:

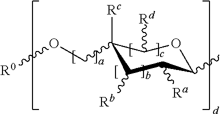

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
each occurrence of R^{x} is as defined for compounds of Formula III; and
LG is a suitable leaving group selected from the group consisting of halogen, imidate, alkoxy, sulphonyloxy, optionally substituted alkylsulphonyl, optionally substituted alkenylsulfonyl, optionally substituted arylsulfonyl, and diazonium moieties;
(c) to give a compound of formula I as described herein.

In some embodiments, the method comprises the steps of:
(a) Providing a compound of Formula IV:

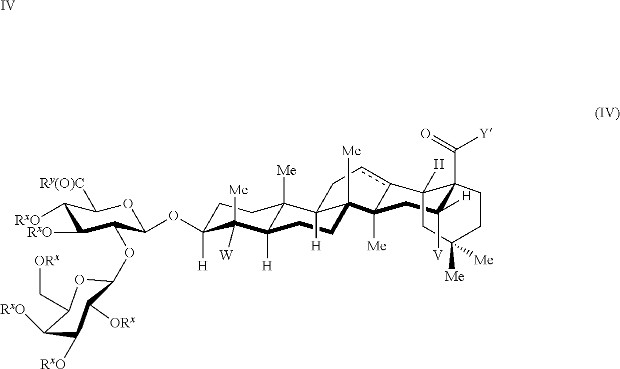

wherein:
[symbol] is a single or double bond;
Y′ is hydrogen, halogen, alkyl, aryl, OR, OR^{y}, OH, NR₂, NR₃⁺, NHR, NH₂, SR, or NROR;
W is Me, —CHO, —CH₂OR^{x}, —C(O)R^{y}, or

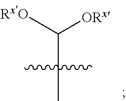

V is hydrogen or —OR^{x};
R^{y} is —OH, or a carboxyl protecting group selected from the group consisting of ester, amides, and hydrazides;
R^{s} is

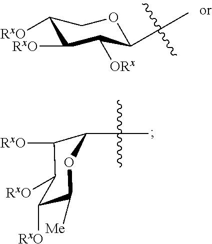

each occurrence of R^{x′} is independently an optionally substituted group selected from 6-10-membered aryl, C1-6 aliphatic, or C₁₋₆ heteroaliphatic having 1-2 heteroatoms independently selected from the group consisting of nitrogen, oxygen, and sulfur; or:
   two R^{x′} are taken together to form a 5-7-membered heterocyclic ring having 1-2 heteroatoms independently selected from the group consisting of nitrogen, oxygen, and sulfur;
each occurrence of R is independently hydrogen, an optionally substituted group selected from acyl, arylalkyl, 6-10-membered aryl, C₁₋₁₂ aliphatic, or C₁₋₁₂ heteroaliphatic having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, and carbonates;
(b) treating said compound of Formula IV under suitable conditions with a compound of formula V:

LG-Z (V)
wherein:
Z is hydrogen; a cyclic or acyclic, optionally substituted moiety selected from the group consisting of acyl, aliphatic, heteroaliphatic, aryl, arylalkyl, and heteroaryl; or a carbohydrate domain having the structure:

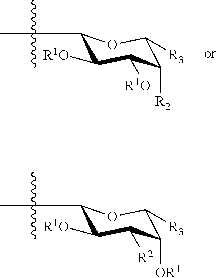

wherein:
each occurrence of R1 is Rx or a carbohydrate domain having the structure:

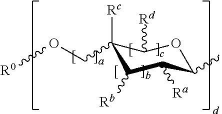

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

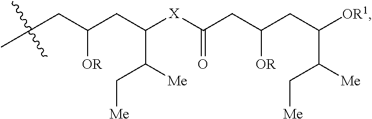

wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR^{1′}, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
R^{1′} is R^{x} or a carbohydrate domain having the structure:

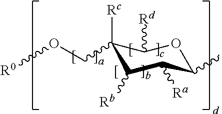

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
each occurrence of R^{x} is as defined for compounds of formula IV; and
LG is a suitable leaving group selected from the group consisting of halogen, imidate, alkoxy, sulphonyloxy, optionally substituted alkylsulphonyl, optionally substituted alkenylsulfonyl, optionally substituted arylsulfonyl, and diazonium moieties;
(c) to give a compound of Formula II as described herein.

According to another aspect of the present subject matter, the compounds disclosed in this application have been shown to be useful as adjuvants. In another aspect, the present application provides a method for preparing compounds according to the embodiments of this application. In another aspect, the present invention provides a method of potentiating an immune response to an antigen, comprising administering to a subject a provided vaccine in an effective amount to potentiate the immune response of said subject to said antigen.

In another aspect, the present invention provides methods of vaccinating a subject, comprising administering a provided vaccine to said subject. In some embodiments, the subject is human. In some embodiments, the vaccine is administered as an injectable.

In another aspect, the invention provides pharmaceutical compositions comprising compounds of the invention and pharmaceutically acceptable excipients. In certain embodiments, the pharmaceutical composition is a vaccine comprising an antigen and an inventive adjuvant.

In another aspect, the invention provides kits comprising pharmaceutical compositions of inventive compounds. In some embodiments, the kits comprise prescribing information. In some embodiments, such kits include the combination of an inventive adjuvant compound and another immunotherapeutic agent. The agents may be packaged separately or together. The kit optionally includes instructions for prescribing the medication. In certain embodiments, the kit includes multiple doses of each agent. The kit may include sufficient quantities of each component to treat a subject for a week, two weeks, three weeks, four weeks, or multiple months. In certain embodiments, the kit includes one cycle of immunotherapy. In certain embodiments, the kit includes a sufficient quantity of a pharmaceutical composition to immunize a subject against an antigen long term.

As used herein, the following definitions shall apply unless otherwise indicated.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-12 aliphatic carbon atoms. In some embodiments, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C3-C6 hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR+ (as in N-substituted pyrrolidinyl)).

The term “unsaturated,” as used herein, means that a moiety has one or more units of unsaturation.

As used herein, the term “bivalent C₁₋₁₂ (or C₁₋₂₆, C₁₋₁₆, C₁₋₈) or saturated or unsaturated, straight or branched, hydrocarbon chain,” refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)n-, wherein n is a positive integer, preferably from 1 to 30, from 1 to 28, from 1 to 26, from 1 to 24, from 1 to 22, from 1 to 20, from 1 to 18, from 1 to 16, from 1 to 14, from 1 to 12, from 1 to 10, from 1 to 8, from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “alkynylene” refers to a bivalent alkynyl group. A substituted alkynylene chain is a polymethylene group containing at least one double bond in which one or more hydrogen atoms are replaced with a substituent. Suitable substituents include those described below for a substituted aliphatic group.

The term “acyl,” used alone or a part of a larger moiety, refers to groups formed by removing a hydroxy group from a carboxylic acid.

The term “halogen” means F, Cl, Br, or I.

The terms “aralkyl” and “arylalkyl” are used interchangeably and refer to alkyl groups in which a hydrogen atom has been replaced with an aryl group. Such groups include, without limitation, benzyl, cinnamyl, and dihyrocinnamyl.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.”

In certain embodiments of the present invention, “aryl” refers to an aromatic ring system which includes, but not limited to, phenyl, biphenyl, naphthyl, anthracyl and the like, which may bear one or more substituents. Also, included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The terms “heteroaralkyl” and “heteroarylalkyl” refer to an alkyl group substituted by a heteroaryl moiety, wherein the alkyl and heteroaryl portions independently are optionally substituted.

The term “heteroaliphatic,” as used herein, means aliphatic groups wherein one or two carbon atoms are independently replaced by one or more of oxygen, sulfur, nitrogen, or phosphorus. Heteroaliphatic groups may be substituted or unsubstituted, branched or unbranched, cyclic or acyclic, and include “heterocycle,” “heterocyclyl,” “heterocycloaliphatic,” or “heterocyclic” groups.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

In another aspect, the present invention provides “pharmaceutically acceptable” compositions, which comprise a therapeutically effective amount of one or more of the compounds described herein, formulated together with one or more pharmaceutically acceptable carriers (additives) and/or diluents. As described in detail, the pharmaceutical compositions of the present invention may be specially formulated for administration by injection.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

In other cases, the compounds of the present invention may contain one or more acidic functional groups and, thus, are capable of forming pharmaceutically-acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary, tertiary, or quaternary amine. Salts derived from appropriate bases include alkali metal, alkaline earth metal, ammonium and N+(C₁₋₄alkyl)4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Representative organic amines useful for the formation of base addition salts include ethylamine, diethylamine, ethylenediamine, ethanolamine, diethanolamine, piperazine and the like. (See, for example, Berge et al., supra).

Unless otherwise stated, structures depicted herein are also meant to include all isomeric (e.g., enantiomeric, diastereomeric, and geometric (or conformational)) forms of the structure; for example, the R and S configurations for each stereocenter, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Provided compounds may comprise one or more saccharide moieties. Unless otherwise specified, both D- and L-configurations, and mixtures thereof, are within the scope of the invention. Unless otherwise specified, both α- and β-linked embodiments, and mixtures thereof, are contemplated by the present invention.

If, for instance, a particular enantiomer of a compound of the present invention is desired, it may be prepared by asymmetric synthesis, chiral chromatography, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. Such compounds are useful, for example, as analytical tools, as probes in biological assays, or as therapeutic agents in accordance with the present invention.

One of ordinary skill in the art will appreciate that the synthetic methods, as described herein, utilize a variety of protecting groups. By the term “protecting group,” as used herein, it is meant that a particular functional moiety, e.g., O, S, or N, is masked or blocked, permitting, if desired, a reaction to be carried out selectively at another reactive site in a multifunctional compound. In preferred embodiments, a protecting group reacts selectively in good yield to give a protected substrate that is stable to the projected reactions; the protecting group is preferably selectively removable by readily available, preferably non-toxic reagents that do not attack the other functional groups; the protecting group forms a separable derivative (more preferably without the generation of new stereogenic centers); and the protecting group will preferably have a minimum of additional functionality to avoid further sites of reaction. As detailed herein, oxygen, sulfur, nitrogen, and carbon protecting groups may be utilized. By way of non-limiting example, hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methyl pentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [241,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)-amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N′,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-di phenyl borinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described by Greene and Wuts (supra).

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph, which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄(O)R^{o}; —C(S)R^{o}; —(CH₂)₀₋₄C(O)OR^{o}; —(CH₂)₀₋₄(O)SR^{o}; —(CH₂)₀₋₄(O)OSiR^{o}₃; —(CH₂)₀₋₄OC(O)R^{o}; —OC(O)(CH₂)₀₋₄SR, —SC(S)SR^{o}; —(CH₂)₀₋₄SC(O)R^{o}; —(CH₂)₀₋₄(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched)alkylene)O—N(R^{o})₂; or —(C₁₋₄ straight or branched)alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6-membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{Δ}, -(haloR^{Δ}), —(CH₂)₀₋₂OH, —(CH₂)₀₋₂OR^{Δ}, —(CH₂)₀₋₂CH(OR^{Δ})₂; —O(haloR^{Δ}), —CN, —N₃, —(CH₂)₀₋₂C(O)R^{Δ}, —(CH₂)₀₋₂C(O)OH, —(CH₂)₀₋₂C(O)OR^{Δ}, —(CH₂)₀₋₂SR^{Δ}, —(CH₂)₀₋₂SH, —(CH₂)₀₋₂NH₂, —(CH₂)₀₋₂NHR^{Δ}, —(CH₂)₀₋₂NR^{Δ}₂, —NO₂, —SiR^{Δ}₃, —OSiR^{Δ}₃, —C(O)SR^{Δ}, —(C₁₋₄ straight or branched alkylene)C(O)OR^{Δ}, or —SSR. wherein each R^{Δ} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{Δ}, -(haloR^{Δ}), —OH, —OR^{Δ}, —O(haloR^{Δ}), —CN, —C(O)OH, —C(O)OR^{Δ}, —NH₂, —NHR^{Δ}, —NR^{Δ}₂, or —NO₂, wherein each R^{Δ} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each Rt is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur. Suitable substituents on the aliphatic group of Rt are independently halogen, —R^{Δ}, -(haloR^{Δ}), —OH, —OR^{Δ}, —O(haloR^{Δ}), —CN, —C(O)OH, —C(O)OR^{Δ}, —NH₂, —NHR^{Δ}, —NR^{Δ}₂, or —NO₂, wherein each R^{Δ} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The term “enriched” as used herein refers to a mixture having an increased proportion of one or more species. In some embodiments, the mixture is “enriched” following a process that increases the proportion of one or more desired species in the mixture. In some embodiments, the desired species comprise(s) greater than 10% of the mixture. In some embodiments, the desired species comprise(s) greater than 25% of the mixture. In some embodiments, the desired species comprise(s) greater than 40% of the mixture. In some embodiments, the desired species comprise(s) greater than 60% of the mixture. In some embodiments, the desired species comprise(s) greater than 75% of the mixture. In some embodiments, the desired species comprise(s) greater than 85% of the mixture. In some embodiments, the desired species comprise(s) greater than 90% of the mixture. In some embodiments, the desired species comprise(s) greater than 95% of the mixture. Such proportions can be measured any number of ways, for example, as a molar ratio, volume to volume, or weight to weight.

The term “pure” refers to compounds that are substantially free of compounds of related non-target structure or chemical precursors (when chemically synthesized). This quality may be measured or expressed as “purity.” In some embodiments, a target compound has less than about 30%, 20%, 10%, 5%, 2%, 1%, 0.5%, and 0.1% of non-target structures or chemical precursors. In certain embodiments, a pure compound of present invention is only one prosapogenin compound (i.e., separation of target prosapogenin from other prosapogenins).

The term “carbohydrate” refers to a sugar or polymer of sugars. The terms “saccharide”, “polysaccharide”, “carbohydrate”, and “oligosaccharide”, may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose. (e.g., 2′-fluororibose, deoxyribose, and hexose). Carbohydrates may exist in many different forms, for example, conformers, cyclic forms, acyclic forms, stereoisomers, tautomers, anomers, and isomers.

Further objects, features, and advantages of the present application will become apparent form the detailed which is set forth below when considered together with the figures of drawing.

### BRIEF DESCRIPTION OF THE DRAWINGS

FIG. 1 depicts the chemical structure of QS-21-Api and QS-21-Xyl. Percentages correspond to the natural abundance of each isomer in isolated extracts of QS-21.

FIG. 2 depicts data showing the immunogenicity of high or low dose Prevnar-13 or of Lym2-CRM197 conjugate in combination with synthetic QS-21 (SQS-21) or Compound 26 (TiterQuil-1-0-5-5/TQL-1055).

FIG. 3 depicts data showing immunogenicity of Adacel alone or in combination with Compound 26 (TiterQuil-1-0-5-5/TQL-1055) or QS-21 (Pharm/tox study).

FIG. 4 depicts data showing immunogenicity of Engerix-B alone or in combination with 10, 30, 100 or 300 mcg of Compound 26 (TiterQuil-1-0-5-5/TQL-1055).

FIG. 5 depicts data showing the hemolytic activity of QS-21 at 2 uM, 5 uM and 20 uM, and Compound 26 (TiterQuil-1-0-5-5/TQL-1055) at 20 uM, 100 uM and 200 uM. % Hemolytic activity reported as % of Triton-X100/SDS lysis control.

FIGS. 6-31 depict H NMR analyses (CDCl₃) of the materials discussed in Example 1.

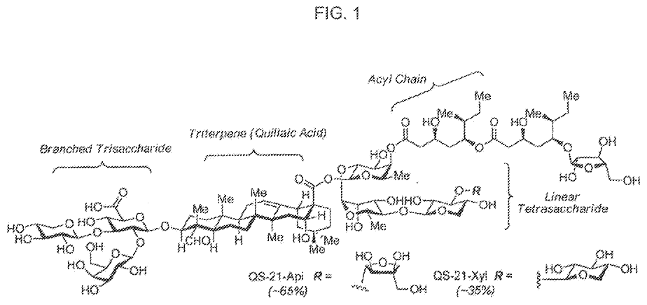

### DETAILED DESCRIPTION OF CERTAIN EMBODIMENTS

The clinical success of anticancer, antiviral and antimicrobial vaccines critically depends on the identification of, and access to, novel potent adjuvants with attenuated toxicity. In this context, specific fractions from extracts of the bark of Quillaja saponaria (QS) have proven to be exceedingly powerful adjuvants in immunotherapy. The QS-21 fraction, comprising isomeric forms of a complex triterpene glycoside saponin had previously been considered the most promising immuno-potentiator in several antitumor (melanoma, breast, small cell lung cancer, prostate) and infectious-disease (HIV, malaria) vaccine therapies.

However, the tolerated dose of QS-21 in cancer patients typically does not exceed 100-150 μg, above which significant local erythema and systemic flu-like symptoms arise. QS-21's inherent instability can lead to toxicities associated with its breakdown. It is also known that QS-21 is hemolytic, and this hemolytic activity had previously been hypothesized that at least some of QS-21's adjuvant activity was related to its hemolytic properties.

The inventors of the present subject matter have found that compounds of the present application, which are in some embodiments synthetic analogues of QS-21 and other QS extraction fractions such as QS-7, possess significant stand-alone adjuvant activity as well as a high degree of tolerability and/or reduced side-effects. These new adjuvant compounds are more cost-effective to produce than natural QS-21, more stable, more efficacious, and less toxic for use in prophylactic and therapeutic vaccination programs. Some embodiments have no detectable toxicity in pharmacology/toxicology studies in mice at doses close to the likely 1000 mcg human dose. Some embodiments are surprisingly completely nonhemolytic while still retaining their adjuvant properties. This is surprising in part because it was initially thought that both QS-21 toxicity and potency were related to hemolysis and other cellular toxicity associated with QS-21. Some embodiments of the present application exhibit greater stability and less hemolytic activity by replacing the unstable ester linkage of the acyl chain in QS-21 with a very stable amide linkage, resulting in adjuvant active analogs of QS-21. Some embodiments also retain adjuvant activity despite having a simplified structure as compared to QS-21, resulting in higher synthetic yields and significantly reduced synthetic steps and cost of manufacture in comparison to synthetic QS-21.

The present application also provides efficient semi-synthetic methods of synthesizing the compounds of the present application, thereby significantly reducing the number of synthetic steps required to access this potent class of adjuvants.

The application also includes pharmaceutical compositions comprising the compounds of the present application together with an immunologically effective amount of an antigen associated with a bacterium or virus. Bacterium or viruses included in the subject matter of this application consist of those associated with Hepatitis B, pneumococcus, diphtheria, tetanus, pertussis, or Lyme disease including the closely related spirochetes of the genus Borrelia such as, B. burgdorferi, B. garinii, B. afzelli, and B. japonica.

The application also includes methods of vaccinating a human patient comprising administering an immunologically effective amount of a pharmaceutical compositions or of the compounds of the present application. The application also includes methods for increasing the immune response to a vaccine comprising administering an immunologically effective amount of a pharmaceutical compositions or of the compounds of the present application.

### Compounds

Compounds of this invention include those described generally above, and are further illustrated by the classes, subclasses, and species disclosed herein. In some embodiments, provided compounds are analogs of naturally occurring triterpene glycoside saponins and intermediates thereto.

### Description of Exemplary Compounds

In some embodiments, provided compounds are analogs of Quillaja saponins. In some embodiments, provided compounds are prosapogenins. In certain embodiments, provided compounds are analogs of QS-7 and QS-21 and possess potent adjuvant activity.

In one aspect, the present application provides compounds of Formula I:

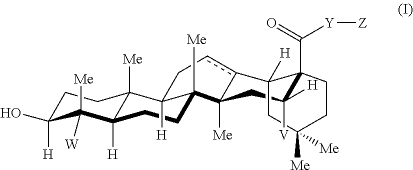

or a pharmaceutically acceptable salt thereof, wherein
[symbol] is a single or double bond;
W is —CHO;
V is hydrogen or OR^{x};
Y is CH₂, —O—, —NR—, or —NH—;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

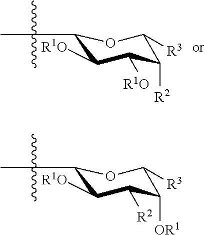

wherein each occurrence of R¹ is R^{x} or a carbohydrate domain having the structure:

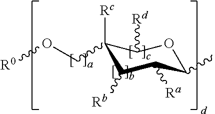

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

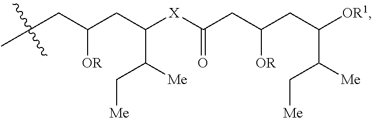

wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, carbamates, and carbonates;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
   two R on the same nitrogen atom are taken with the nitrogen atom to form a 4-7-membered heterocyclic ring having 1-2 heteroatoms independently selected from the group consisting of nitrogen, oxygen, and sulfur.

In one aspect the present application provides compounds of Formula

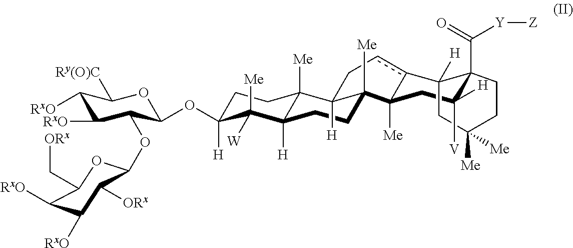

or a pharmaceutically acceptable salt thereof, wherein
[symbol] is a single or double bond;
W is Me, —CHO, or

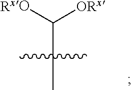

V is hydrogen or OR^{x};
Y is CH₂, —O—, —NR—, or —NH—;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

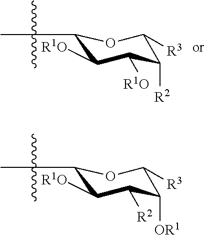

wherein each occurrence of R¹ is R^{x} or a carbohydrate domain having the structure:

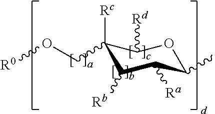

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

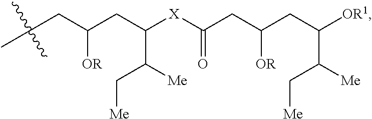

wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, carbamates, and carbonates;
R^{y} is —OH, —OR, or a carboxyl protecting group selected from the group consisting of ester, amides, and hydrazides;
R^{s} is

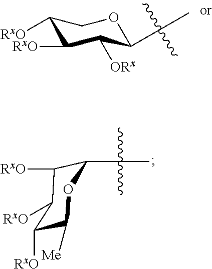

each occurrence of R^{x′} is independently an optionally substituted group selected from 6-10-membered aryl, C₁₋₆ aliphatic, or C₁₋₆ heteroaliphatic having 1-2 heteroatoms independently selected from the group consisting of nitrogen, oxygen, and sulfur; or:
   two R^{x′} are taken together to form a 5-7-membered heterocyclic ring having 1-2 heteroatoms independently selected from the group consisting of nitrogen, oxygen, and sulfur;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
   two R on the same nitrogen atom are taken with the nitrogen atom to form a 4-7-membered heterocyclic ring having 1-2 heteroatoms independently selected from the group consisting of nitrogen, oxygen, and sulfur.

In one aspect, the present application provides compounds of Formula I:

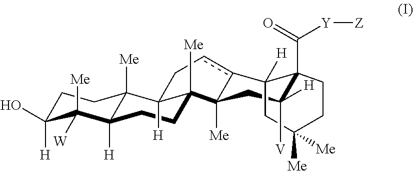

or a pharmaceutically acceptable salt thereof, wherein [symbol]
is a single or double bond;

### W is —CHO;

### V is —OH;

### Y is —O—;

wherein Z is a carbohydrate domain having the structure:

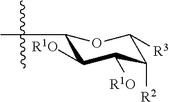

wherein:
R¹ is independently H or

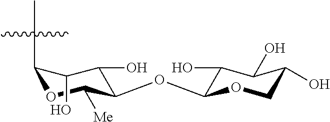

R² is NHR⁴;
R³ is CH₂OH; and
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

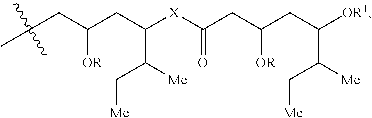

wherein:
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur.

It will be appreciated by one of ordinary skill in the art that the compounds of the present application include but are not necessarily limited to those compounds encompassed in the genus definitions set forth as part of the present section. The compounds encompassed by this application include at least all of the compounds disclosed in the entire specification as a whole, including all individual species within each genus.

In certain embodiments, V is OR^{x}. In certain embodiments V is OH. In certain embodiments, V is H.

In certain embodiments, Y is —O—. In certain embodiments, Y is —NH—. In certain embodiments, Y is —NR—. In certain embodiments, Y is CH₂.

In certain embodiments, Z is hydrogen. In certain embodiments, Z is a cyclic or acyclic, optionally substituted moiety. In certain embodiments, Z is an acyl. In certain embodiments, Z is an aliphatic. In certain embodiments, Z is a heteroaliphatic. In certain embodiments, Z is aryl. In certain embodiments Z is arylalkyl. In certain embodiments, Z is heteroacyl. In certain embodiments, Z is heteroaryl. In certain embodiments, Z is a carbohydrate domain having the structure:

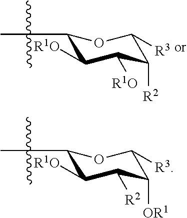

In some embodiments Z is a carbohydrate domain having the structure:

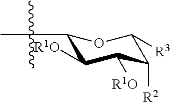

wherein:
R¹ is independently H or

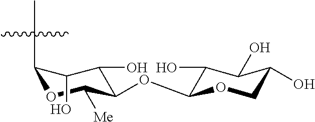

R² is NHR⁴,
R³ is CH₂OH, and
R⁴ is selected from:

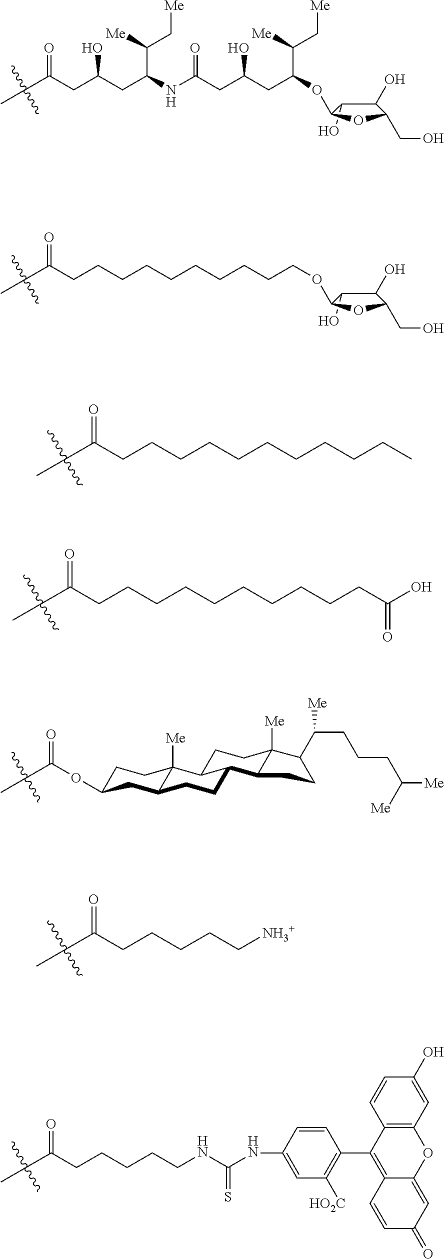

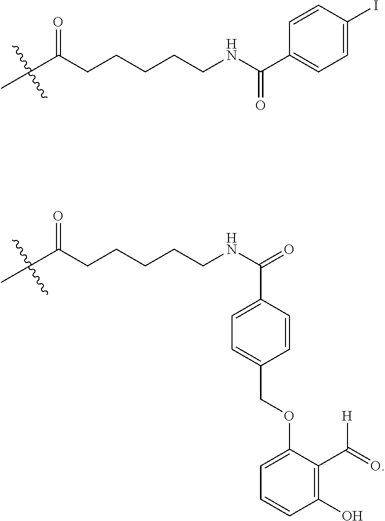 [SMILES: *C(=O)CCCCCNC(=O)c1ccc(I)cc1 ; *C(=O)CCCCCNC(=O)c1ccc(COc2cccc(O)c2C=O)cc1]

In some embodiments, R¹ is R^{x}. In other embodiments, R¹ a carbohydrate domain having the structure:

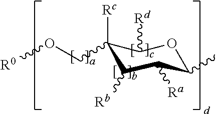

In some aspects, each occurrence of a, b, and c is independently 0, 1, or 2. In some embodiments, d is an integer from 1-5. In some embodiments, each d bracketed structure may be the same. In some embodiments, each d bracketed structure may be different. In some embodiments, the d bracketed structure represents a furanose or a pyranose moiety. In some embodiments, and the sum of b and c is 1 or 2.

In some embodiments, R⁰ is hydrogen. In some embodiments, R⁰ is an oxygen protecting group selected from the group. In some embodiments, R⁰ is an alkyl ether. In some embodiments, R⁰ is a benzyl ether. In some embodiments, R⁰ is a silyl ether. In some embodiments, R⁰ is an acetal. In some embodiments, R⁰ is ketal. In some embodiments, R⁰ is an ester. In some embodiments, R⁰ is a carbamate. In some embodiments, R⁰ is a carbonate. In some embodiments, R⁰ is an optionally substituted moiety. In some embodiments, R⁰ is an acyl. In some embodiments, R⁰ is a C₁₋₁₀ aliphatic. In some embodiments, R⁰ is a C₁₋₆ heteroaliphatic. In some embodiments, R⁰ is a 6-10-membered aryl. In some embodiments, R⁰ is a arylalkyl. In some embodiments, R⁰ is a 5-10 membered heteroaryl having 1-4 heteroatoms independently selected from nitrogen, oxygen, or sulfur. In some embodiments, R⁰ is a 4-7 membered heterocyclyl having 1-2 heteroatoms independently selected from the group consisting of nitrogen, oxygen, and sulfur.

In some embodiments, R^{a} is hydrogen. In some embodiments, R^{a} is a halogen. In some embodiments, R^{a} is OH. In some embodiments, R^{a} is OR. In some embodiments, R^{a} is OR^{x}. In some embodiments, R^{a} is NR₂. In some embodiments, R^{a} is NHCOR. In some embodiments, R^{a} an acyl. In some embodiments, R^{a} is C₁₋₁₀ aliphatic. In some embodiments, R^{a} is C₁₋₆ heteroaliphatic. In some embodiments, R^{a} is 6-10-membered aryl. In some embodiments, R^{a} is arylalkyl. In some embodiments, R^{a} is 5-10-membered heteroaryl having 1-4 heteroatoms independently selected from nitrogen, oxygen, sulfur. In some embodiments, R^{a} is 4-7-membered heterocyclyl having 1-2 heteroatoms independently selected from the group consisting of nitrogen, oxygen, and sulfur.

In some embodiments, R^{b} is hydrogen. In some embodiments, R^{b} is a halogen. In some embodiments, R^{b} is OH. In some embodiments, R^{b} is OR. In some embodiments, R^{b} is OR^{x}. In some embodiments, R^{b} is NR₂. In some embodiments, R^{b} is NHCOR. In some embodiments, R^{b} an acyl. In some embodiments, R^{b} is C₁₋₁₀ aliphatic. In some embodiments, R^{b} is C₁₋₆ heteroaliphatic. In some embodiments, R^{b} is 6-10-membered aryl. In some embodiments, R^{b} is arylalkyl. In some embodiments, R^{b} is 5-10-membered heteroaryl having 1-4 heteroatoms independently selected from nitrogen, oxygen, sulfur. In some embodiments, R^{b} is 4-7-membered heterocyclyl having 1-2 heteroatoms independently selected from the group consisting of nitrogen, oxygen, and sulfur.

In some embodiments, R^{b} is hydrogen. In some embodiments, R^{b} is a halogen. In some embodiments, R^{b} is OH. In some embodiments, R^{b} is OR. In some embodiments, R^{b} is OR^{x}. In some embodiments, R^{b} is NR₂. In some embodiments, R^{b} is NHCOR. In some embodiments, R^{b} an acyl. In some embodiments, R^{b} is C₁₋₁₀ aliphatic. In some embodiments, R^{b} is C₁₋₆ heteroaliphatic. In some embodiments, R^{b} is 6-10-membered aryl. In some embodiments, R^{b} is arylalkyl. In some embodiments, R^{b} is 5-10-membered heteroaryl having 1-4 heteroatoms independently selected from nitrogen, oxygen, sulfur. In some embodiments, R^{b} is 4-7-membered heterocyclyl having 1-2 heteroatoms independently selected from the group consisting of nitrogen, oxygen, and sulfur.

In some embodiments, R^{c} is hydrogen. In some embodiments, R^{c} is a halogen. In some embodiments, R^{c} is OH. In some embodiments, R^{c} is OR. In some embodiments, R^{c} is OR^{x}. In some embodiments, R^{c} is NR₂. In some embodiments, R^{c} is NHCOR. In some embodiments, R^{c} an acyl. In some embodiments, R^{c} is C₁₋₁₀ aliphatic. In some embodiments, R^{c} is C₁₋₆ heteroaliphatic. In some embodiments, R^{c} is 6-10-membered aryl. In some embodiments, R^{c} is arylalkyl. In some embodiments, R^{c} is 5-10-membered heteroaryl having 1-4 heteroatoms independently selected from nitrogen, oxygen, sulfur. In some embodiments, R^{c} is 4-7-membered heterocyclyl having 1-2 heteroatoms independently selected from the group consisting of nitrogen, oxygen, and sulfur.

In some embodiments, R^{d} is hydrogen. In some embodiments, R^{d} is a halogen. In some embodiments, R^{d} is OH. In some embodiments, R^{d} is OR. In some embodiments, R^{d} is OR^{x}. In some embodiments, R^{d} is NR₂. In some embodiments, R^{d} is NHCOR. In some embodiments, R^{d} an acyl. In some embodiments, R^{d} is C₁₋₁₀ aliphatic. In some embodiments, R^{d} is C₁₋₆ heteroaliphatic. In some embodiments, R^{d} is 6-10-membered aryl. In some embodiments, R^{d} is arylalkyl. In some embodiments, R^{d} is 5-10-membered heteroaryl having 1-4 heteroatoms independently selected from nitrogen, oxygen, sulfur. In some embodiments, R^{d} is 4-7-membered heterocyclyl having 1-2 heteroatoms independently selected from the group consisting of nitrogen, oxygen, and sulfur.

In some embodiments, R² is hydrogen. In some embodiments, R² is a halogen. In some embodiments, R² is OH. In some embodiments, R² is OR. In some embodiments, R² is OC(O)R⁴. In some embodiments, R² is OC(O)OR⁴. In some embodiments, R² is OC(O)NHR⁴. In some embodiments, R² is OC(O)NRR⁴. In some embodiments, R² is OC(O)SR⁴. In some embodiments, R² is NHC(O)R⁴. In some embodiments, R² is NRC(O)R⁴. In some embodiments, R² is NHC(O)OR⁴. In some embodiments, R² is NHC(O)NHR⁴. In some embodiments, R² is NHC(O)NRR⁴. In some embodiments, R² is NHR⁴. In some embodiments, R² is N(R⁴)₂. In some embodiments, R² is NHR⁴. In some embodiments, R² is NRR⁴. In some embodiments, R² is N₃. In some embodiments, R² is C₁₋₁₀ aliphatic. In some embodiments, R² is C₁₋₆ heteroaliphatic. In some embodiments, R² is 6-10-membered aryl. In some embodiments, R² is arylalkyl. In some embodiments, R² is 5-10 membered heteroaryl having 1-4 heteroatoms independently selected from the group consisting of nitrogen, oxygen, and sulfur. In some embodiments, R² is 4-7-membered heterocyclyl having 1-2 heteroatoms independently selected from the group consisting of nitrogen, oxygen, and sulfur.

In some embodiments, R³ is hydrogen. In some embodiments, R³ is a halogen. In some embodiments, R³ is CH₂OR¹. In some embodiments, R³ is an acyl. In some embodiments, R³ is C₁₋₁₀ aliphatic. In some embodiments, R³ is C₁₋₆ heteroaliphatic. In some embodiments, R³ is 6-10-membered aryl. In some embodiments, R³ is arylalkyl. In some embodiments, R³ is 5-10-membered heteroaryl having 1-4 heteroatoms independently selected from the group consisting of nitrogen, oxygen, and sulfur. In some embodiments, R³ is 4-7-membered heterocyclyl having 1-2 heteroatoms independently selected from the group consisting of nitrogen, oxygen, and sulfur.

In some embodiments, R⁴ is -T-R^{z}. In some embodiments, R⁴ is —C(O)-T-R^{z}. In some embodiments, R⁴ is —NH-T-R^{z}. In some embodiments, R⁴ is —O-T-R^{z}. In some embodiments, R⁴ is —S-T-R^{z}. In some embodiments, R⁴ is —C(O)NH-T-R^{z}. In some embodiments, R⁴ is C(O)O-T-R^{z}. In some embodiments, R⁴ is C(O)S-T-R^{z}. In some embodiments, R⁴ is C(O)NH-T-O-T-R^{z}. In some embodiments, R⁴ is —O-T-R^{z}. In some embodiments, R⁴ is -T-O-T-R^{z}. In some embodiments, R⁴ is -T-S-T-R^{z}. In some embodiments, R⁴ is

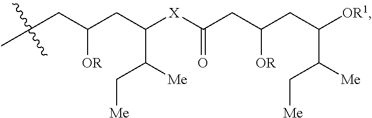

In some embodiments, X is —O—. In some embodiments, X is —NR—. In some embodiments, X is T-R^{z}.

In some embodiments, T is a covalent bond or a bivalent C₁₋₂₆ saturated or unsaturated, straight or branched, aliphatic or heteroaliphatic chain.

In some embodiments, R^{z} is hydrogen. In some embodiments, R^{z} is a halogen. In some embodiments, R^{z} is —OR. In some embodiments, R^{z} is —OR^{x}. In some embodiments, R^{z} is —OR¹. In some embodiments, R^{z} is —OR^{1′}. In some embodiments, R^{z} is —SR. In some embodiments, R^{z} is NR₂. In some embodiments, R^{z} is —C(O)OR. In some embodiments, R^{z} is —C(O)R. In some embodiments, R^{z} is —NHC(O)R. In some embodiments, R^{z} is —NHC(O)OR. In some embodiments, R^{z} is NC(O)OR. In some embodiments, R^{z} is an acyl. In some embodiments, R^{z} is arylalkyl. In some embodiments, R^{z} is heteroarylalkyl. In some embodiments, R^{z} is C₁₋₆ aliphatic. In some embodiments, R^{z} is 6-10-membered aryl. In some embodiments, R^{z} is 5-10-membered heteroaryl having 1-4 heteroatoms independently selected from nitrogen, oxygen, or sulfur. In some embodiments, R^{z} is 4-7-membered heterocyclyl having 1-2 heteroatoms independently selected from the group consisting of nitrogen, oxygen, and sulfur.

In some embodiments, R^{x} is hydrogen. In some embodiments, R^{x} is an oxygen protecting group. In some embodiments, R^{x} is an alkyl ether. In some embodiments, R^{x} is a benzyl ether. In some embodiments, R^{x} is silyl ether. In some embodiments, R^{x} is an acetal. In some embodiments, R^{x} is ketal. In some embodiments, R^{x} is ester. In some embodiments, R^{x} is carbamate. In some embodiments, R^{x} is carbonate.

In some embodiments, R^{y} is —OH. In some embodiments, R^{y} is —OR. In some embodiments, R^{y} is a carboxyl protecting group. In some embodiments, R^{y} is an ester. In some embodiments, R^{y} is an amide. In some embodiments, R^{y} is a hydrazide.

In some embodiments, R⁵ is

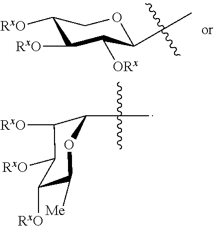

In some embodiments, R^{x′} is optionally substituted 6-10-membered aryl. In some embodiments, R^{x′} is optionally substituted C₁₋₆ aliphatic. In some embodiments, R^{x′} is optionally substituted or C₁₋₆ heteroaliphatic having 1-2 heteroatoms independently selected from the group consisting of nitrogen, oxygen, and sulfur. In some embodiments, two R^{x′} are taken together to form a 5-7-membered heterocyclic ring having 1-2 heteroatoms independently selected from the group consisting of nitrogen, oxygen, and sulfur.

In some embodiments, R is hydrogen. In some embodiments, R is an acyl. In some embodiments, R is arylalkyl. In some embodiments, R is 6-10-membered aryl. In some embodiments, R is C₁₋₆ aliphatic. In some embodiments, R is C₁₋₆ heteroaliphatic having 1-2 heteroatoms independently selected from the group consisting of nitrogen, oxygen, and sulfur. In some embodiments, two R on the same nitrogen atom are taken with the nitrogen atom to form a 4-7-membered heterocyclic ring having 1-2 heteroatoms independently selected from the group consisting of nitrogen, oxygen, and sulfur.

In some embodiments, R^{1′} has the same embodiments as R¹.

Exemplary compounds of Formula I are set forth in Table 1 below:

TABLE 1
EXEMPLARY COMPOUNDS OF FORMULA I

| | |
|---|---|
| 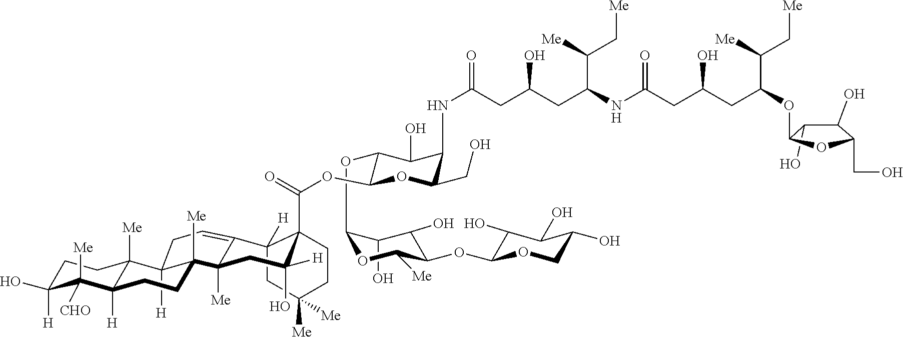 | I-1 |
| 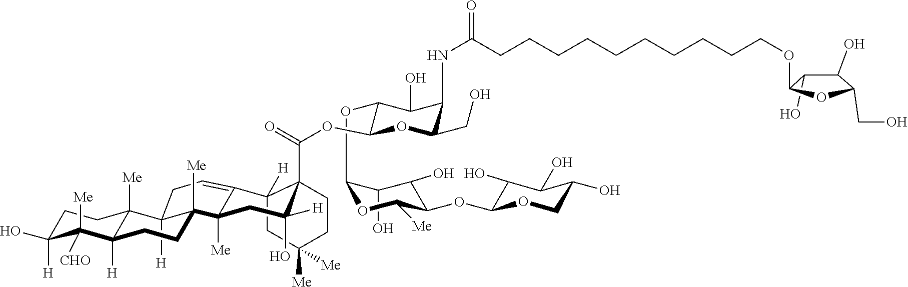 | I-2 |
| 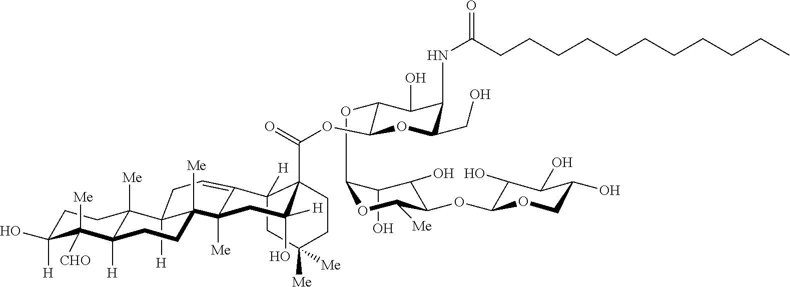 | I-3 |
| 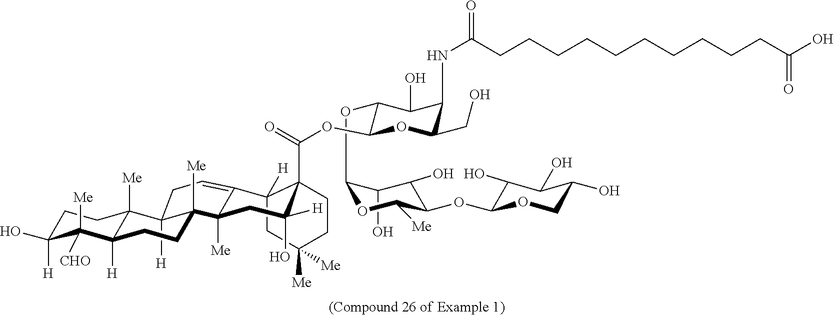 | I-4 |
| 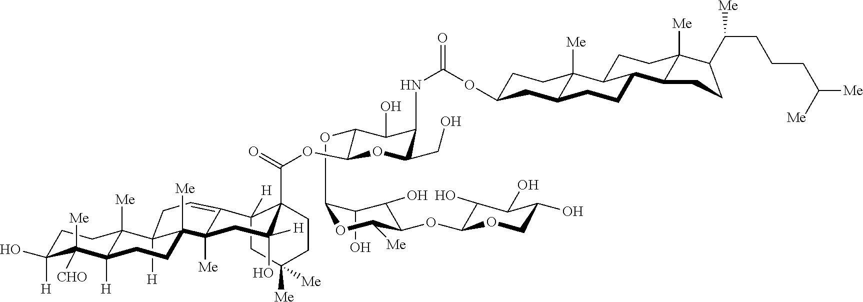 | I-5 |
| 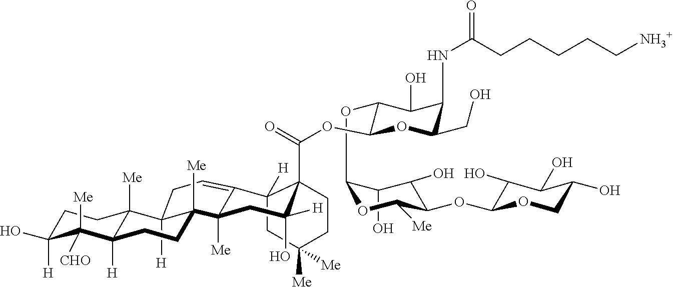 | I-6 |
| 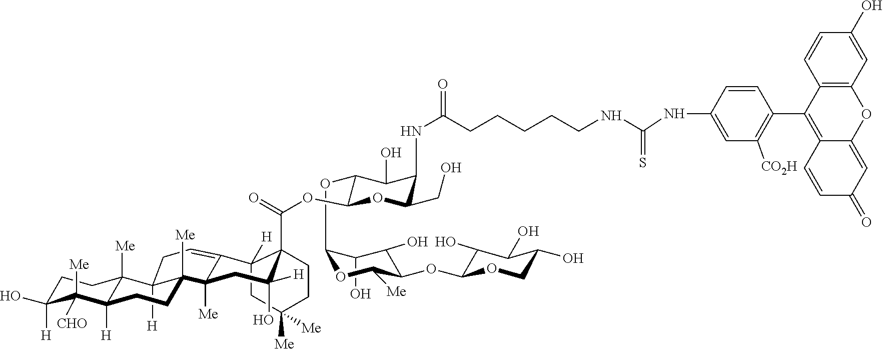 | I-7 |
| 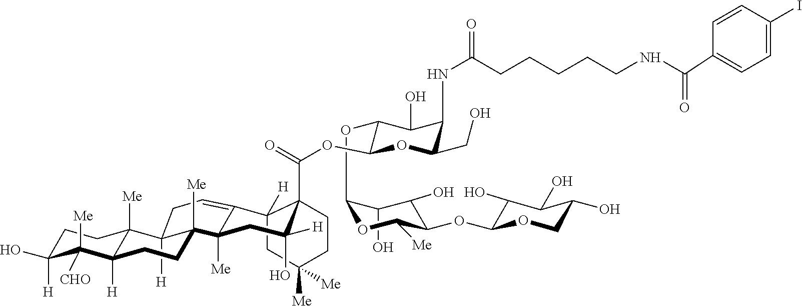 | I-8 |
| 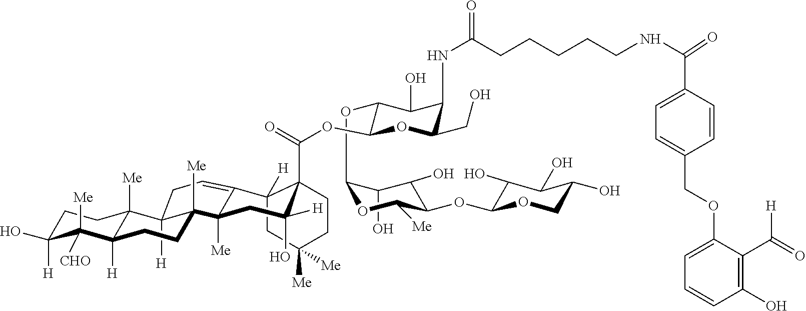 | I-9 |

It will be appreciated that it is not an object of the present subject matter to claim compounds disclosed in the prior art that are the result of isolation or degradation studies on naturally occurring prosapogenins or saponins.

### Synthesis of Compounds

As described in U.S. Ser. No. 12/420,803, issued as U.S. Pat. No. 8,283,456 (and its parent/child U.S. applications and publications), the synthesis of QS-21 and at least some of its analogues can be carried out in part by obtaining semi-purified abstract from Quillaja saponaria (commercially available as Quil-A, Accurate Chemical and Scientific Corporation, Westbury, N.Y.) comprising a mixture of at least 50 distinct saponin species (van Setten, D. C.; Vandewerken, G.; Zomer, G.; Kersten, G. F. A. Rapid Commun. Mass Spectrom. 1995, 9, 660-666). Many of said saponin species include a triterpene-trisaccharide substructure as found in immunologically-active Quillaja saponins such as QS-21 and QS-7. Exposing these saponin species to base hydrolysis affords a mixture enriched with prosapogenins A, B, and C (shown below).

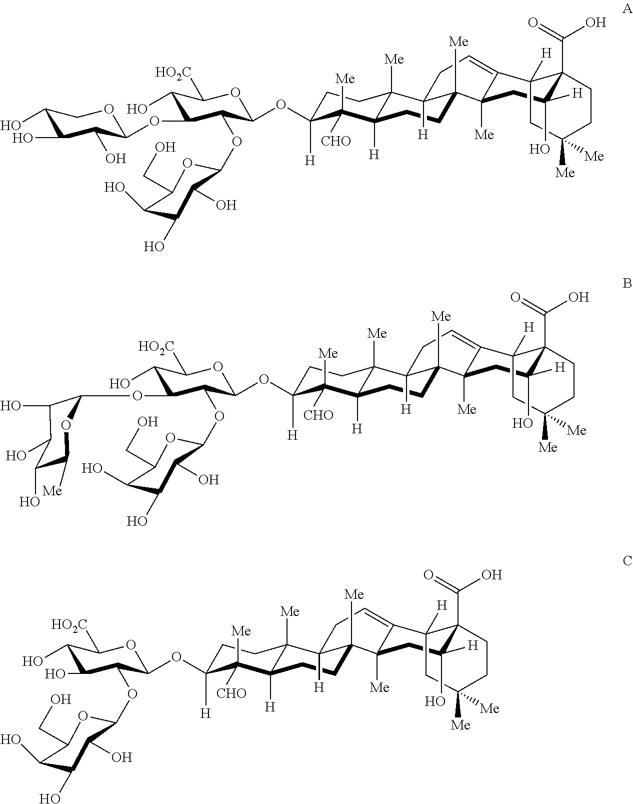

U.S. Ser. No. 12/420,803, issued as U.S. Pat. No. 8,283,456 (and its parent/child U.S. applications and publications) presents a strategy that allows for the facile separation of derivatized prosapogenins A, B, and C via silica gel chromatography. It will be appreciated that some embodiments of the present application may be synthesized in part using the methods described in U.S. Ser. No. 12/420,803, issued as U.S. Pat. No. 8,283,456 (and its parent/child U.S. applications and publications), particularly the methods relating to facile separation of derivatized prosapogenins A, B, and C. In one aspect, separated derivatized prosapogenins A, B, and/or C may then be used to synthesize QS-21 or analogs thereof using the methods described herein.

In one embodiment, the present application provides semi-synthetic methods for synthesizing QS-7, QS-21, and related analogs, the method comprising coupling a triterpene compound with a compound comprising a saccharide to form a compound of Formula I or of Formula II. In some embodiments, the method comprises the steps of:
(a) Providing a compound of Formula III:

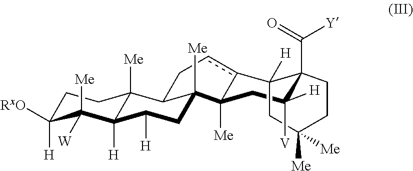

wherein:
[symbol] is a single or double bond;
Y′ is hydrogen, halogen, alkyl, aryl, OR, OR^{y}, OH, NR₂, NR₃⁺, NHR, NH₂, SR, or NROR;
W is Me, —CHO, —CH₂OR^{x}, —C(O)R^{y}, or

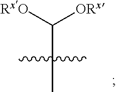

V is hydrogen or —OR^{x};
R^{y} is —OH, or a carboxyl protecting group selected from the group consisting of ester, amides, and hydrazides;
each occurrence of R^{x′} is independently an optionally substituted group selected from 6-10-membered aryl, C1-6 aliphatic, or C₁₋₆ heteroaliphatic having 1-2 heteroatoms independently selected from the group consisting of nitrogen, oxygen, and sulfur; or:
   two R^{x′} are taken together to form a 5-7-membered heterocyclic ring having 1-2 heteroatoms independently selected from the group consisting of nitrogen, oxygen, and sulfur;
each occurrence of R is independently hydrogen, an optionally substituted group selected from acyl, arylalkyl, 6-10-membered aryl, C₁₋₁₂ aliphatic, or C₁₋₁₂ heteroaliphatic having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, and carbonates;
(b) treating said compound of Formula III under suitable conditions with a compound of formula V:

LG-Z (V)
wherein:
Z is hydrogen; a cyclic or acyclic, optionally substituted moiety selected from the group consisting of acyl, aliphatic, heteroaliphatic, aryl, arylalkyl, and heteroaryl; or a carbohydrate domain having the structure:

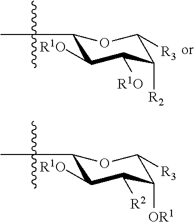

wherein:
each occurrence of R1 is Rx or a carbohydrate domain having the structure:

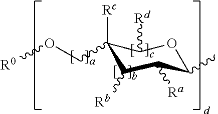

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

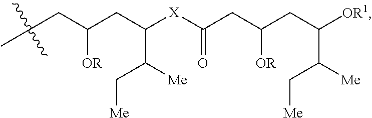

wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is as defined for compounds of formula III; and
LG is a suitable leaving group selected from the group consisting of halogen, imidate, alkoxy, sulphonyloxy, optionally substituted alkylsulphonyl, optionally substituted alkenylsulfonyl, optionally substituted arylsulfonyl, and diazonium moieties;
(c) to give a compound of Formula I as described herein.

In some embodiments, the method comprises the steps of:
(a) Providing a compound of Formula IV:

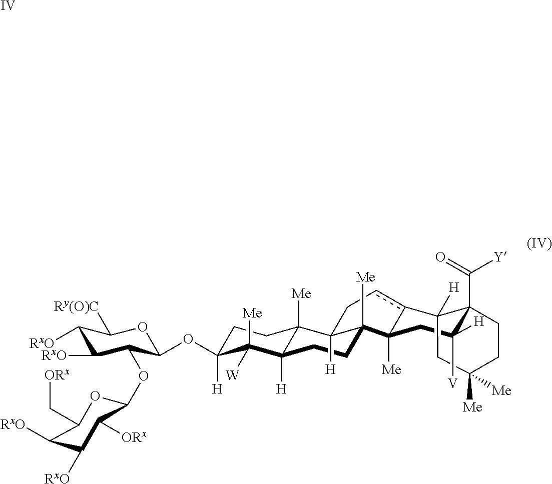

wherein:
[symbol] is a single or double bond;
Y′ is hydrogen, halogen, alkyl, aryl, OR, OR^{y}, OH, NR₂, NR₃⁺, NHR, NH₂, SR, or NROR;
W is Me, —CHO, —CH₂OR^{x}, —C(O)R^{y}, or

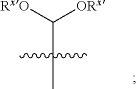

V is hydrogen or —OR^{x};
R^{y} is —OH, or a carboxyl protecting group selected from the group consisting of ester, amides, and hydrazides;
R^{s} is

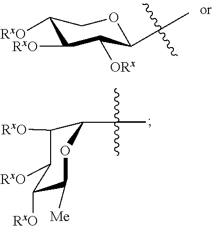

each occurrence of R^{x′} is independently an optionally substituted group selected from 6-10-membered aryl, C1-6 aliphatic, or C₁₋₆ heteroaliphatic having 1-2 heteroatoms independently selected from the group consisting of nitrogen, oxygen, and sulfur; or:
   two R^{x′} are taken together to form a 5-7-membered heterocyclic ring having 1-2 heteroatoms independently selected from the group consisting of nitrogen, oxygen, and sulfur;
each occurrence of R is independently hydrogen, an optionally substituted group selected from acyl, arylalkyl, 6-10-membered aryl, C₁₋₁₂ aliphatic, or C₁₋₁₂ heteroaliphatic having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, and carbonates;
(b) treating said compound of Formula IV under suitable conditions with a compound of formula V:

LG-Z (V)
wherein:
Z is hydrogen; a cyclic or acyclic, optionally substituted moiety selected from the group consisting of acyl, aliphatic, heteroaliphatic, aryl, arylalkyl, and heteroaryl; or a carbohydrate domain having the structure:

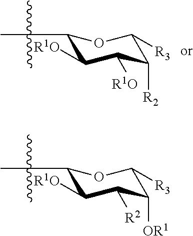

wherein:
each occurrence of R1 is Rx or a carbohydrate domain having the structure:

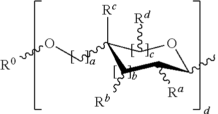

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

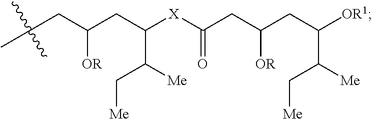

wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is as defined for compounds of formula IV; and
LG is a suitable leaving group selected from the group consisting of halogen, imidate, alkoxy, sulphonyloxy, optionally substituted alkylsulphonyl, optionally substituted alkenylsulfonyl, optionally substituted arylsulfonyl, and diazonium moieties;
(c) to give a compound of formula II as described herein.

In another aspect, the present application provides a synthesis method comprising:
(a) providing a compound of Formula III:

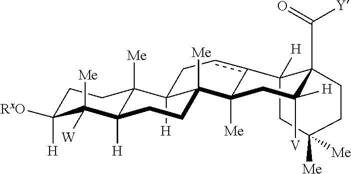

### (III)

wherein:
[symbol] is a single or double bond;
Y′ is hydrogen, halogen, alkyl, aryl, OR, OR^{y}, OH, NR₂, NR₃⁺, NHR, NH₂, SR, or NROR;
W —CHO;
V —OR^{x};
R^{x} is independently hydrogen or an oxygen protecting group selected from the group consisting of alkyl ethers, benzyl ethers, silyl ethers, acetals, ketals, esters, carbamates, and carbonates;
(b) treating said compound of Formula III under suitable conditions with a compound of formula V:

LG-Z (V)
wherein:
Z is a carbohydrate domain having the structure:

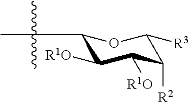

wherein:
R¹ is independently H or

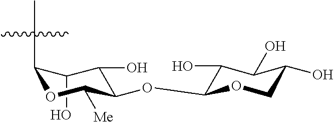

R² is NHR⁴;
R³ is CH₂OH; and
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

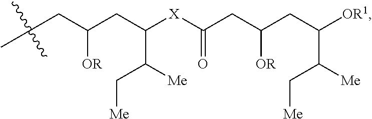

wherein:
   X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
(c) to give a compound of Formula I as described herein.

In another aspect, the present application provides a method of synthesizing a compound of Formula I, or an intermediate thereof, comprising the following steps:
(a) providing a compound of Formula III:

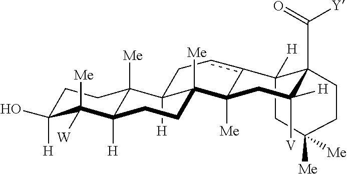

### (III)

wherein:
[symbol] is a single or double bond;
Y′ is hydrogen, halogen, alkyl, aryl, OR, OR^{y}, OH, NR₂, NR₃⁺, NHR, NH₂, SR, or NROR;
W —CHO;
V —OH;
wherein one or more substituents of the compound of Formula III are optionally protected;
(b) reacting the compound of Formula III with a compound of Formula X:

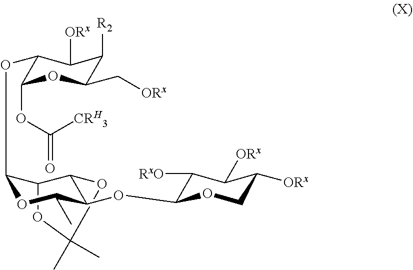

wherein:
R^{H} is a halogen;
R² is hydrogen, N₃, NH₂, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

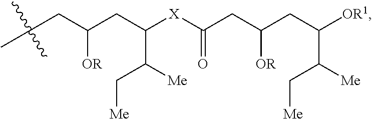

wherein:
X is —O—, —NR—, or T-R^{z};
T is a covalent bond or a bivalent C₁₋₂₆ saturated or unsaturated, straight or branched, aliphatic or heteroaliphatic chain;
R^{z} is hydrogen, halogen, —OR, —OR^{x}, —OR^{1′}, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
R^{x} is independently hydrogen or an oxygen protecting group selected from the group consisting of alkyl ethers, benzyl ethers, silyl ethers, acetals, ketals, esters, carbamates, and carbonates; and
R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
   two R on the same nitrogen atom are taken with the nitrogen atom to form a 4-7-membered heterocyclic ring having 1-2 heteroatoms independently selected from the group consisting of nitrogen, oxygen, and sulfur;
R^{1′} is R^{x} or a carbohydrate domain having the structure:

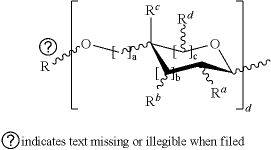

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur.

In one embodiment, the compound of Formula X is:

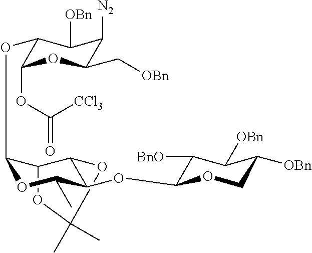

In one embodiment, the method includes reacting the product of step (b) or a further downstream product with R⁴—OH. In one embodiment, the method includes reacting the product of step (b) or a compound obtained after modifying the product of step (b) with R⁴—OH. In one embodiment, the method includes reacting the product of step (b) or a compound obtained after modifying the product of step (b) with R⁴—OH. In one embodiment, the method includes reacting the product of step (b) or an intermediate with R⁴—OH. In one embodiment, R⁴—OH is HO—C(O)—(CH₂)₁₀—C(O)—OR^{x}. In one embodiment, R^{x} is H. In one embodiment, R^{x} is Bn.

In another aspect, the present application discloses a synthesis route for Compound 26 (TQL-1055/TiterQuil-1-0-5-5), as shown in Example 1. It will be understood by one of ordinary skill in the art that the synthesis of Compound 26 and its intermediates described in these figures may be modified or adapted according to the knowledge of one of ordinary skill in the art to obtain other molecules. It will be understood by one of ordinary skill in the art that the synthesis of Compound 26 and its intermediates described in these figures may be modified or adapted according to the knowledge of one of ordinary skill in the art to after the route to Compound 26 (TQL-1055/TiterQuil-1-0-5-5).

In another aspect of the subject matter, synthesis of QS-21, QS-7, and/or analogs of these compounds may be undertaken by using one or more of the methods disclosed in the examples, including Examples 1 and 2, described in this application. Although the synthesis of several compounds is disclosed in these examples, one of ordinary skill in the art will appreciate that these methods may be modified or adapted according to the knowledge of one of ordinary skill in the art to obtain other molecules.

In another aspect, the present application also includes methods for obtaining the compounds according the present application comprising providing a compound according to the application and a second substance, and subsequently purifying the compound of the application by removing at least a portion of the second substance.

In another aspect, the present application includes methods for obtaining synthesis intermediates of compounds according to the present application from soapwort plants or soapwort seeds.

### Adjuvants

Most protein and glycoprotein antigens are poorly immunogenic or non-immunogenic when administered alone. Strong adaptive immune responses to such antigens often requires the use of adjuvants. Immune adjuvants are substances that, when administered to a subject, increase the immune response to an antigen or enhance certain activities of cells from the immune system. An adjuvant may also allow the use of a lower dose of antigen to achieve a useful immune response in a subject.

Common adjuvants include alum, Freund's adjuvant (an on-in-water emulsion with dead mycobacteria), Freund's adjuvant with MDP (an oil-in-water emulsion with muramyl dipeptide, MDP, a constituent of mycobacteria), alum plus Bordetella pertussis (aluminum hydroxide gel with killed B. pertussis). Such adjuvants are thought to act by delaying the release of antigens and enhancing uptake by macrophages. Immune stimulatory complexes (ISCOMs) are open cage-like complexes typically with a diameter of about 40 nm that are built up by cholesterol, lipid, immunogen, and saponin such as Quil-A (a Quillaja saponin extract). ISCOMs deliver antigen to the cytosol, and have been demonstrated to promote antibody response and induction of T helper cell as well as cytotoxic T lymphocyte responses in a variety of experimental animal models.

Natural saponin adjuvant QS-21 is far more potent than currently used adjuvants, like alum. QS-21's superiority over more than 20 other adjuvants tested in preclinical models and over 7 other adjuvants used in the clinic has been demonstrated. Thus, QS-21 has been widely used despite its three major liabilities: dose limiting toxicity, poor stability, and the limited availability of quality product.

Use of QS-21 as an adjuvant has been associated with notable adverse biological effects. In humans, QS-21 has displayed both local and systemic toxicity. Maximum doses for cancer patients are 100-150 μg and for healthy patients are typically 50 μg (an immunology suboptimal dose). As a result, clinical success of non-cancer vaccines depends upon the identification of novel, potent adjuvants that are more tolerable.

The present application encompasses the recognition that synthetic access to and structural modification of QS-21 and related Quillaja saponins may afford compounds with high adjuvant potency and low toxicity, as well as having more stability and being more cost effective.

### Vaccines

Compositions in this application are useful as vaccines to induce active immunity towards antigens in subjects. Any animal that may experience the beneficial effects of the compositions of the present application is within the scope of subjects that may be treated. In some embodiments, the subjects are mammals. In some embodiments, the subjects are humans.

The vaccines of the present application may be used to confer resistance to infection by either passive or active immunization. When the vaccines of the present application are used to confer resistance through active immunization, a vaccine of the present application is administered to an animal to elicit a protective immune response which either prevents or attenuates a proliferative or infectious disease. When the vaccines of the present application are used to confer resistance to infection through passive immunization, the vaccine is provided to a host animal (e.g., human, dog, or mouse), and the antisera elicited by this vaccine is recovered and directly provided to a recipient suspected of having an infection or disease or exposed to a causative organism.

The present application thus concerns and provides a means for preventing or attenuating a proliferative disease resulting from organisms which have antigens that are recognized and bound by antisera produced in response to the immunogenic antigens included in vaccines of the present application. As used herein, a vaccine is said to prevent or attenuate a disease if its administration to an animal results either in the total or partial attenuation (i.e., suppression) of a symptom or condition of the disease, or in the total or partial immunity of the animal to the disease.

The administration of the vaccine (or the antisera which it elicits) may be for either a “prophylactic” or “therapeutic” purpose. When provided prophylactically, the vaccine(s) are provided in advance of any symptoms of proliferative disease. The prophylactic administration of the vaccine(s) serves to prevent or attenuate any subsequent presentation of the disease. When provided therapeutically, the vaccine(s) is provided upon or after the detection of symptoms which indicate that an animal may be infected with a pathogen. The therapeutic administration of the vaccine(s) serves to attenuate any actual disease presentation. Thus, the vaccines may be provided either prior to the onset of disease proliferation (so as to prevent or attenuate an anticipated infection) or after the initiation of an actual proliferation.

Thus, in one aspect the present application provides vaccines comprising an antigen associated with Hepatitis B, pneumococcus, diphtheria, tetanus, pertussis, or Lyme disease including the closely related spirochetes of the genus Borrelia such as, B. burgdorferi, B. garinii, B. afzelli, and B. japonica.

One of ordinary skill in the art will appreciate that vaccines may optionally include a pharmaceutically acceptable excipient or carrier. Thus, according to another aspect, provided vaccines may comprise one or more antigens that are optionally conjugated to a pharmaceutically acceptable excipient or carrier. In some embodiments, said one or more antigens are conjugated covalently to a pharmaceutically acceptable excipient. In other embodiments, said one or more antigens are non-covalently associated with a pharmaceutically acceptable excipient.

As described above, adjuvants may be used to increase the immune response to an antigen. According to the present application, provided vaccines may be used to invoke an immune response when administered to a subject. In certain embodiments, an immune response to an antigen may be potentiated by administering to a subject a provided vaccine in an effective amount to potentiate the immune response of said subject to said antigen.

### Formulations

The compounds of the present application may be combined with a pharmaceutically acceptable excipient to form a pharmaceutical composition. In certain embodiments, formulations of the present application include injectable formulations. In certain embodiments, the pharmaceutical composition includes a pharmaceutically acceptable amount of a compound of the present application. In certain embodiments, the compounds of the application and an antigen form an active ingredient. In certain embodiments, the compound of the present application alone forms an active ingredient. The amount of active ingredient(s) which can be combined with a carrier material to produce a single dosage form will vary depending upon the host being treated, and the particular mode of administration. The amount of active ingredient(s) that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10°/h to about 30%, or from about 1% to 99%, preferably from 10% to 90%, 20% to 80%, 30% to 70%, 40% to 60%, 45% to 55%, or about 50%.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Non-limiting examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Non-limiting examples of suitable aqueous and nonaqueous carriers, which may be employed in the pharmaceutical compositions of the present application include water, alcohols (including but not limited to methanol, ethanol, butanol, etc.), polyols (including but not limited to glycerol, propylene glycol, polyethylene glycol, etc.), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain additives such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a formulation, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form.

Regardless of the route of administration selected, the compounds of the present application, which may be used in a suitable hydrated form, and/or the pharmaceutical compositions of the present application, are formulated into pharmaceutically-acceptable dosage forms by conventional methods known to those of skill in the art.

### Combinations

Adjuvant formulations have resulted from the mixture of different adjuvants in the same formulation. As a general rule, two or more adjuvants with different mechanisms of action are combined to enhance the potency and type of the immune response to the vaccine antigen.

For example, triterpene glycoside saponin-derived adjuvants of the present invention can be formulated in combination with other adjuvants such as Lipid A to increase immunogenicity. One of them, 3-O-desacyl-4″-monophosphoryl lipid A (MPL), is derived from cell wall lipopolysaccharide (LPS) of the Gram-negative Salmonella minnesota R595 strain and is detoxified by mild hydrolytic treatment and purification. MPL demonstrates drastically reduced toxicity compared with the parent LPS molecule, while retaining its adjuvant effect. It is a very powerful stimulator of the immune system, known to act as a TLR4 agonist. Similarly, the present invention can be formulated with alum salts. Saponins as described herein maybe used as a part of immunostimulatory complexes (ISCOMS). ISCOMS are virus like particles of 30-40 nm and dodecahedric structure, composed by Quil A, lipids and cholesterol. Antigens can be inserted in the membrane or encapsulated. A wide variety of proteins have been inserted in these cage-like structures. ISCOMS can be used through the oral, respiratory and vaginal routes. ISCOMS are particularly effective in activating cellular immunity and cytotoxic T cells, but often have problems with stability and toxicity.

One or more of the following are possible combination with triterpene glycoside saponin-derived adjuvants of the present invention: aluminium salts, squalene, monophosphoryl lipid A, MF59 oil-in-water emulsion.

### Dosage

Actual dosage levels of the active ingredients in the pharmaceutical compositions of the present application may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present application employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the present application employed in the pharmaceutical composition at levels lower than that required to achieve the desired therapeutic effect and then gradually increasing the dosage until the desired effect is achieved.

In some embodiments, a compound or pharmaceutical composition of the present application is provided to a subject chronically. Chronic treatments include any form of repeated administration for an extended period of time, such as repeated administrations for one or more months, between a month and a year, one or more years, or longer. In many embodiments, a chronic treatment involves administering a compound or pharmaceutical composition of the present application repeatedly over the life of the subject. Preferred chronic treatments involve regular administrations, for example one or more times a day, one or more times a week, or one or more times a month. In general, a suitable dose, such as a daily dose of a compound of the present application, will be that amount of the compound that is the lowest dose effective to produce a therapeutic effect. Such an effective dose will generally depend upon the factors described above.

Generally, doses of the compounds of the present application for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. However, lower or higher doses can be used. In some embodiments, the dose administered to a subject may be modified as the physiology of the subject changes due to age, disease progression, weight, or other factors.

In some embodiments, provided adjuvant compounds of the present application are administered as pharmaceutical compositions or vaccines. In certain embodiments, it is contemplated that the amount of adjuvant compound administered will be 1-2000 μg. In certain embodiments, it is contemplated that the amount of adjuvant compound administered will be 1-1000 μg. In certain embodiments, it is contemplated that the amount of adjuvant compound administered will be 1-500 μg. In certain embodiments, it is contemplated that the amount of adjuvant compound administered will be 1-250 μg. In certain embodiments, it is contemplated that the amount of adjuvant compound administered will be 100-1000 μg. In certain embodiments, it is contemplated that the amount of adjuvant compound administered will be 100-500 μg. In certain embodiments, it is contemplated that the amount of adjuvant compound administered will be 100-200 μg. In certain embodiments, it is contemplated that the amount of adjuvant compound administered will be 250-500 μg. In certain embodiments, it is contemplated that the amount of adjuvant compound administered will be 10-1000 μg. In certain embodiments, it is contemplated that the amount of adjuvant compound administered will be 500-1000 μg. In certain embodiments, it is contemplated that the amount of adjuvant compound administered will be 50-250 μg. In certain embodiments, it is contemplated that the amount of adjuvant compound administered will be 50-500 μg.

In some embodiments, provided adjuvant compounds of the present application are administered as pharmaceutical compositions or vaccines. In certain embodiments, it is contemplated that the amount of adjuvant compound administered will be 1-2000 mg. In certain embodiments, it is contemplated that the amount of adjuvant compound administered will be 1-1000 mg. In certain embodiments, it is contemplated that the amount of adjuvant compound administered will be 1-500 mg. In certain embodiments, it is contemplated that the amount of adjuvant compound administered will be 1-250 mg. In certain embodiments, it is contemplated that the amount of adjuvant compound administered will be 100-1000 mg. In certain embodiments, it is contemplated that the amount of adjuvant compound administered will be 100-500 mg. In certain embodiments, it is contemplated that the amount of adjuvant compound administered will be 100-200 mg. In certain embodiments, it is contemplated that the amount of adjuvant compound administered will be 250-500 mg. In certain embodiments, it is contemplated that the amount of adjuvant compound administered will be 10-1000 ma. In certain embodiments, it is contemplated that the amount of adjuvant compound administered will be 500-1000 mg. In certain embodiments, it is contemplated that the amount of adjuvant compound administered will be 50-250 mg. In certain embodiments, it is contemplated that the amount of adjuvant compound administered will be 50-500 mg. In certain embodiments, it is contemplated that the amount of adjuvant compound administered will be 0.01-215.4 mg.

In certain embodiments, it is contemplated that the amount of adjuvant administered will be 1000-5000 μg/kg. In certain embodiments, it is contemplated that the amount of adjuvant administered will be 1000-4000 μg/kg. In certain embodiments, it is contemplated that the amount of adjuvant administered will be 1000-3000 μg/kg. In certain embodiments, it is contemplated that the amount of adjuvant administered will be 1000-2000 μg/kg. In certain embodiments, it is contemplated that the amount of adjuvant administered will be 2000-5000 μg/kg. In certain embodiments, it is contemplated that the amount of adjuvant administered will be 2000-4000 μg/kg. In certain embodiments, it is contemplated that the amount of adjuvant administered will be 2000-3000 μg/kg. In certain embodiments, it is contemplated that the amount of adjuvant administered will be 3000-5000 μg/kg. In certain embodiments, it is contemplated that the amount of adjuvant administered will be 3000-4000 μg/kg. In certain embodiments, it is contemplated that the amount of adjuvant administered will be 4000-5000 μg/kg. In certain embodiments, it is contemplated that the amount of adjuvant administered will be 1-500 μg/kg. In certain embodiments, it is contemplated that the amount of adjuvant administered will be 500-1000 μg/kg. In certain embodiments, it is contemplated that the amount of adjuvant administered will be 1000-1500 μg/kg. In certain embodiments, it is contemplated that the amount of adjuvant administered will be 1 mg/kg. In certain embodiments, it is contemplated that the amount of adjuvant administered will be 2 mg/kg. In certain embodiments, it is contemplated that the amount of adjuvant administered will be 3 mg/kg. In certain embodiments, it is contemplated that the amount of adjuvant administered will be 4 mg/kg. In certain embodiments, it is contemplated that the amount of adjuvant administered will be 5 mg/kg. In certain embodiments, it is contemplated that the amount of adjuvant administered will be 0.0029-5 mg/kg. In certain embodiments, the amount of adjuvant administered in females is less than the amount of adjuvant administered in males. In certain embodiments, the amount of adjuvant administered to infants is less than the amount of adjuvant administered to adults. In certain embodiments, the amount of adjuvant administered to pediatric recipients is less than the amount of adjuvant administered to adults. In certain embodiments, the amount of adjuvant administered to immunocompromised recipients is more than the amount of adjuvant administered to healthy recipients. In certain embodiments, the amount of adjuvant administered to elderly recipients is more than the amount of adjuvant administered to non-elderly recipients.

If desired, the effective dose of the active compound may be administered as two, three, four, five, six or more sub-doses administered separately at appropriate intervals throughout the day, optionally, in unit dosage forms.

While it is possible for a compound of the present application to be administered alone, in certain embodiments the compound is administered as a pharmaceutical formulation or composition as described above.

The compounds according to the present application may be formulated for administration in any convenient way for use in human or veterinary medicine, by analogy with other pharmaceuticals.

The present application provides kits comprising pharmaceutical formulations or compositions of a compound of the present application. In certain embodiments, such kits include the combination of a compound of formulae I and/or II and an antigen. The agents may be packaged separately or together. The kit optionally includes instructions for prescribing the medication. In certain embodiments, the kit includes multiple doses of each agent. The kit may include sufficient quantities of each component to treat one or more subject for a week, two weeks, three weeks, four weeks, or multiple months. The kit may include a full cycle of immunotherapy. In some embodiments, the kit includes a vaccine comprising one or more bacterial or viral-associated antigens, and one or more provided compounds.

### EXAMPLES

### Example 1: Complete Synthesis of TQL-1055 (Compound 1-4)

It would be understood by one of ordinary skill in the art that common reaction intermediates shown in Examples 1 and 2, and/or protected or modified versions thereof, can be produced according to the schemes shown in either example. Additionally, it is within the level of ordinary skill in the art to modify or adapt the reactions shown in Examples 1 and 2 in order to produce compounds encompassing Formula I or Formula II as described in the present application.

Compound 1

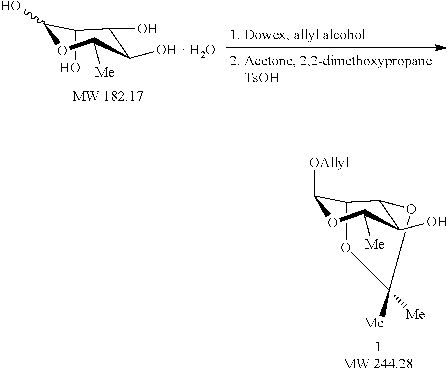

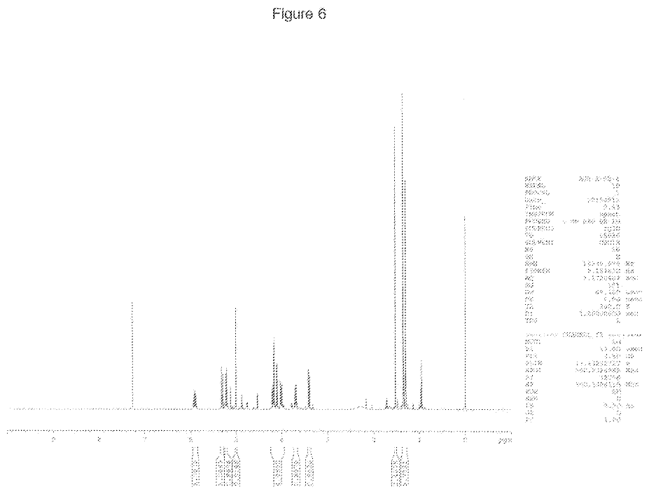

Dowex resin 50WX8 hydrogen form resin (50 g, 1.0 wt.) was placed in a beaker and stirred with allyl alcohol (100 mL, 2 vol.) for about 10 minutes and then filtered. L-rhamnose monohydrate (50 g, 274.5 mmol, 1.0 equiv.), filtered Dowex resin (50 g, 1.0 wt.), and allyl alcohol (400 mL, 8 vol.) was charged into a 1-L 3-neck round bottom flask. The reaction mixture was heated to 90° C. and stirred overnight. The TLC analysis (2:1 DCM/MeOH, CAM stain) showed a small amount of starting material (Rf 0.4). The reaction mixture was cooled to ambient temperature, filtered, and washed with acetone (2×50 mL, 2×1 vol.). The filtrate was concentrated to dryness and co-evaporated with toluene (2×100 mL, 2×2 vol.) to give a black residue (92.2 g). The residue was diluted with acetone (200 mL, 4 vol.). 2,2-Dimethoxypropane (135 mL, 2.7 vol.) and tosic acid monohydrate (0.5 g, 0.01 wt.) were added to the residue, and stirred at ambient temperature overnight. TLC analysis (1:1 heptanes/EtOAc, CAM stain) showed compound 1 was observed (Rf 0.6). Reaction mixture was quenched with Et₃N (20 mL), and then concentrated to dryness to give the crude compound 1 (106.6 g). The crude compound was column purified by CombiFlash (0-35% EtOAc/heptanes) to give pure compound 1 (36.1 g, 53.9% yields) as a yellow-orange oil. The ¹H NMR analysis (CDCl₃) of the prepared material was shown in FIG. 6.

Compound 2

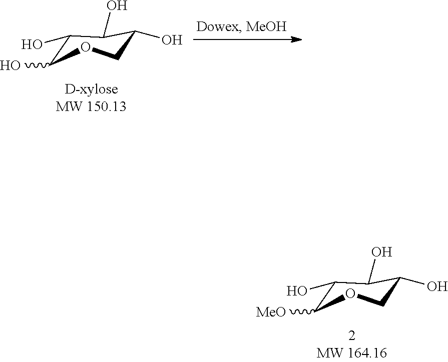

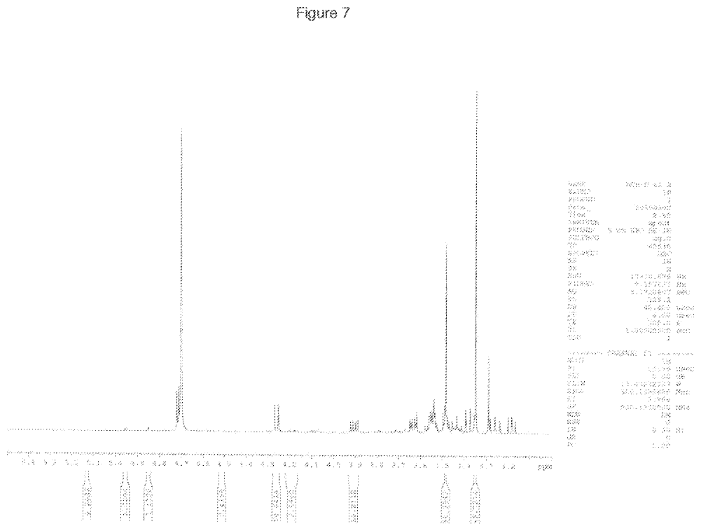

Dowex resin (26.5 g, 0.53 wt, Dowex 50WX8, hydrogen form, 50-100 mesh, Acros) was stirred in MeOH (50 mL) for 10 min and then filtered. To a 2-L 3-neck flask was charged D-xylose (50 g, 333 mmol, 1.0 equiv.), the filtered Dowex resin, and MeOH (665 mL, 13 vol). The reaction mixture was heated to 65° C. and stirred, monitored by ¹H NMR (D₂O). After overnight reaction (21.5 hours) the reaction mixture was cooled to room temperature and filtered. The filtrate was concentrated by rotary evaporator at 40° C., and then dried on high vacuum to give compound 2 as an off-white waxy solid (56.8 g, 100% yields). The ¹H NMR analysis (D₂O) of the prepared material was shown in FIG. 7.

Compound 3

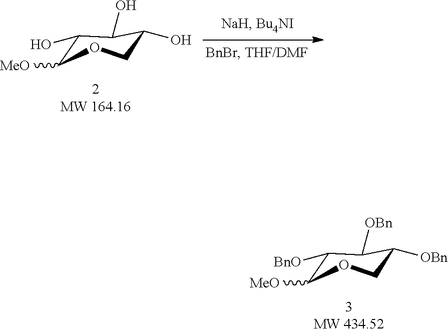

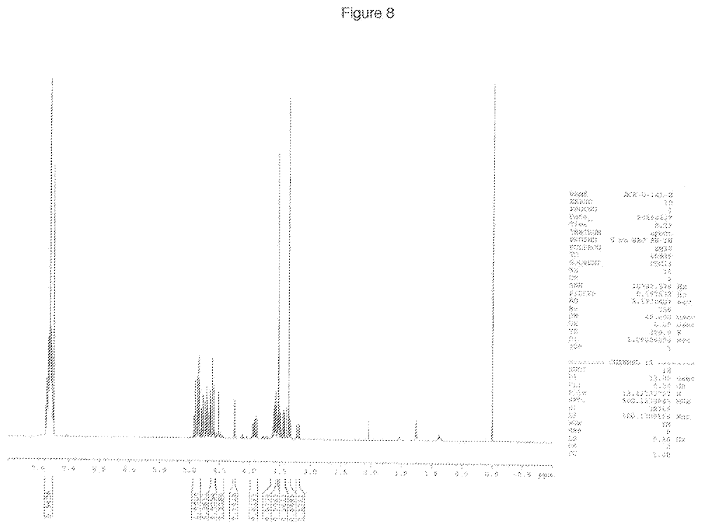

To a 3-L 3-neck flask was charged compound 2 (50.0 g, 305 mmol, 1.0 equiv.), followed by THF (500 mL, 10 vol) and DMF (500 mL, 10 vol). The reaction mixture was cooled in an ice-water bath (temperature=5° C.). Sodium hydride (60% dispersion in oil, 43.9 g, 3.6 equiv.) was slowly added in portions over 20 min. Tetrabutylammonium iodide (22.5 g, 0.2 equiv) was added to the reaction mixture. Benzyl bromide (144.7 mL, 4 equiv.) was slowly added to the flask over 10 min; the reaction was exothermic. Reaction mixture was stirred overnight while slowly warming to room temperature. The mixture was again cooled in an ice/water bath (temperature=5.6° C.), which made the reaction mixture thicker. Iced H₂O (92.5 mL) was slowly drop-wise added to quench the reaction (exothermic). The reaction mixture was stirred for 15 min at 0-10° C. Iced H₂O (1150 mL) was further slowly added to the reaction mixture (exothermic). The reaction was stirred for another 15 min. The mixture was split into 3 1-L portions. Each portion was extracted with EtOAc (2×250 mL). The organic layers were combined, concentrated by rotary evaporator, and dried by high vacuum. The crude product (208.7 g, thick dark orange oil) was split into 4 equal portions. Each portion was purified by CombiFlash (330 g column, 0-10% EtOAc/heptanes). The fractions containing the product were collected (TLC 1:4 EtOAc/heptanes, CAM stain, product Rf 0.3 and 0.4) to give compound 3 (108.8 g, 81% yields) as a light yellow oil. The ¹H NMR analysis (CDCl₃) of the prepared material was shown in FIG. 8.

Compound 4

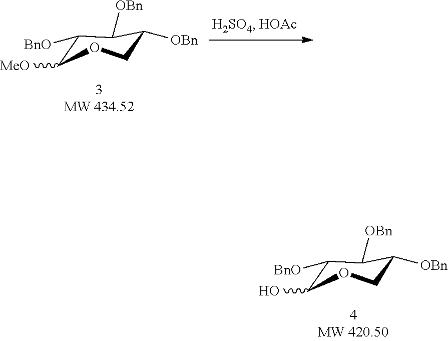

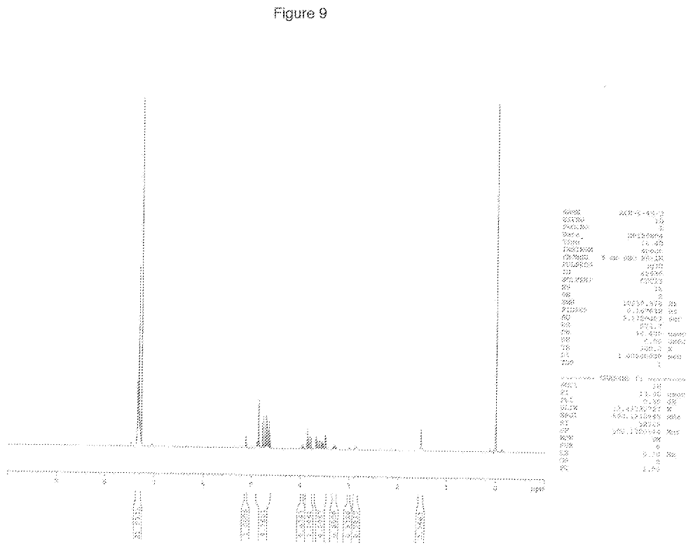

Compound 3 (83.1 g, 191.2 mmol, 1.0 equiv.) was dissolved in acetic acid (924 mL, 11 vol) and charged to a 5-L 3-neck flask. The mixture was heated to 50° C. 2 N aqueous sulfuric acid (125 mL, 1.5 vol) was added and the temperature was increased to 90° C. After 5 hours at 90° C. the TLC analysis showed the starting material was completely consumed and compound 4 was observed (1:4 EtOAc/heptanes; CAM stain; product Rf 0.1). The heating was stopped and the reaction mixture was cooled to room temperature (dark brown solution). DI H₂O (2327 mL, 28 vol) was added drop-wise to the reaction mixture to give a light brown slurry. The mixture was cooled to 0-10° C. and stirred for 1.5 hours. The mixture was filtered off and the filter cake was washed with water (623 mL, 7.5 vol). The solids were dried on high vacuum overnight to give 67.1 g of a light tan solid. This solid was dissolved in toluene (200 mL), and heptanes (1000 mL) was added slowly. The resultant slurry was stirred overnight and then filtered. The filter cake was washed with (1:5) toluene/heptanes (300 mL), and then dried on high vacuum to give compound 4 as an off-white solid (39.9 g, 50% yields). The ¹H NMR analysis (CDCl₃) of the prepared material was shown in FIG. 9.

Compound 5

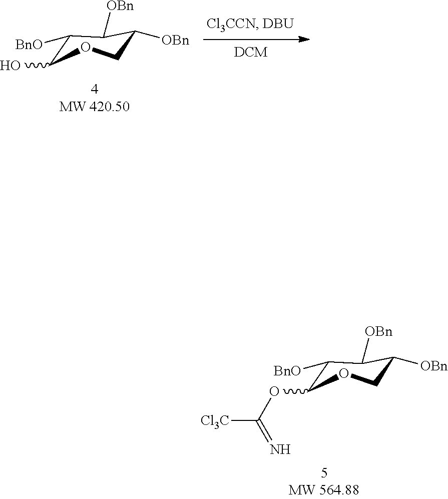

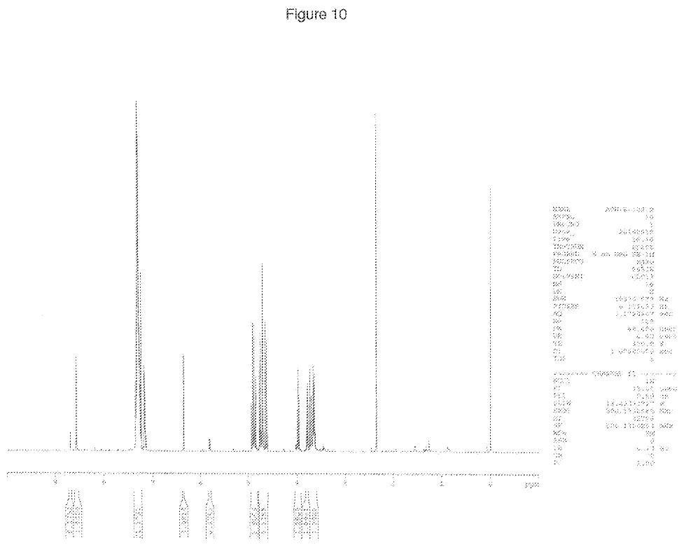

Compound 4 (57.8 g, 137.4 mmol, 1.0 equiv.) was dissolved in DCM (1444 mL, 25 vol) and charged to a 3-L 3-neck flask. The reaction mixture was cooled to <5° C. DBU (27.1 mL, 1.3 equiv.) and Cl₃CCN (137.7 mL, 10 equiv.) was added to the flask. Reaction mixture was stirred at <5° C. After 3 hours, the TLC analysis (TLC plates were pre-treated with 10% Et₃N/heptanes; eluent: heptanes:ethyl acetate 3:1 with 2% Et₃N, CAM stain) showed little starting material (Rf 0.2) and compound 5 was observed (Rf 0.5). The reaction mixture was diluted with toluene (1733 mL, 30 vol) and washed with DI H₂O (3×404 mL, 3×7 vol) and saturated brine (3×289 mL, 3×5 vol). The organic layer was dried over MgSO4, filtered, washed with toluene, and concentrated. A mixture of heptane/EtOAc/Et₃N (15:5:1) was made. The residue was dissolved with 250 mL of the mixture and passed through a plug of silica gel (60 g, 1 wt.) that was pre-treated with 10% Et₃N/heptanes. The plug was washed with the mixture of heptane/EtOAc/Et₃N (15:5:1) until all the desired product was eluted out. The filtrate was concentrated at ambient temperature and dried on high vacuum to give compound 5 as a light orange oil (71.9 g, 93% yields). The ¹H NMR analysis (CDCl₃) of the prepared material was shown in FIG. 10.

Compound 6

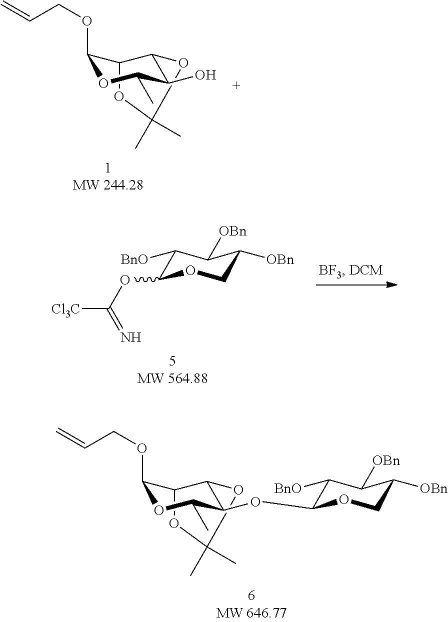

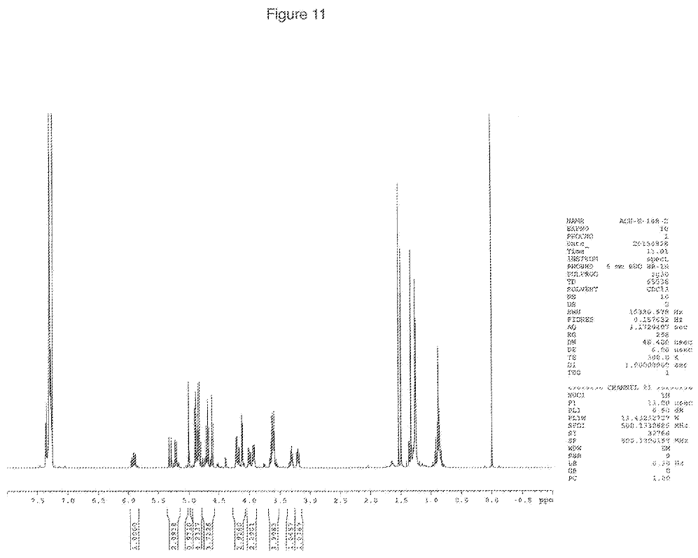

Compound 5 (51.9 g, 91.8 mmol, 1.0 equiv) and compound 1 (24.7 g, 101.0 mmol, 1.1 equiv) was solvent-swapped with toluene and then dissolved in CH₂Cl₂ (1930 mL, 37 vol). The reaction mixture was cooled to −40 to −35° C. using a dry ice/acetone bath. BF₃.OEt₂ (2.3 mL, 0.2 equiv) was added slowly dropwise, which turned the mixture from yellow to an orange color. After 2.5 hours the TLC analysis (6:1 heptanes/EtOAc, CAM stain) showed little starting material (Rf 0.1) and compound 6 was observed (Rf 0.3). The reaction mixture was quenched with Et₃N (38 mL) at <−40° C. and warmed to ambient temperature. The mixture was concentrated to dryness and the residue was purified by CombiFlash (330 g column, 0-10% EtOAc/heptanes) to give compound 6 (34.8 g, 59% yields) as a clear oil. The ¹H NMR analysis (CDCl₃) of the prepared material was shown in FIG. 11.

Compound 7

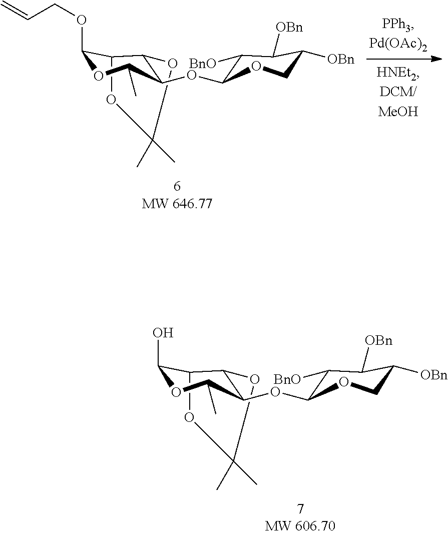

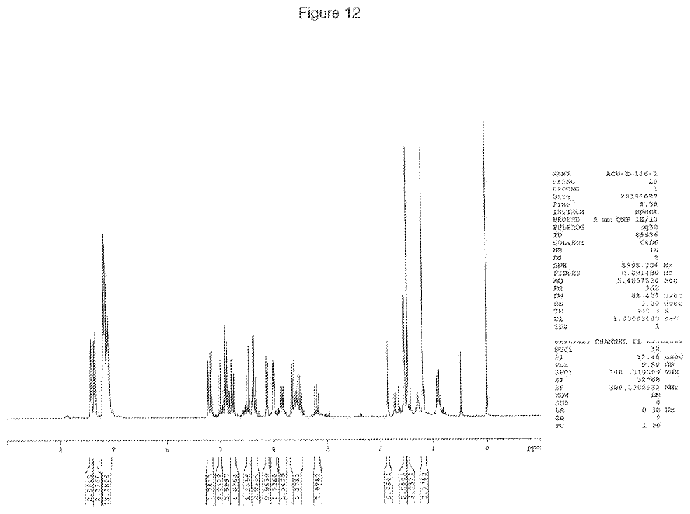

DCM (485 mL, 10 vol) and MeOH (970 mL, 20 vol) were charged into a 3-L 3-neck flask under nitrogen. The mixture was bubbled with nitrogen for about 3 minutes. PPh₃ (23.6 g, 1.2 equiv.), Pd(OAc)₂ (5.05 g, 0.3 equiv), and diethylamine (94 mL, 12.1 equiv.) were added into the 3-L flask. To another flask was added compound 6 (48.5 g, 75.0 mmol) and DCM (242 mL, 5 vol) and was bubbled with nitrogen for about 1 minute. The compound 6 solution in DCM was then charged into the 3 L flask. The mixture was heated to 30° C. while stirring to afford a bright yellow slurry. After 2.5 hours the TLC analysis (3:1 heptanes/EtOAc, CAM stain) showed little starting material (Rf 0.4) and compound 7 was observed (Rf 0.2). The reaction mixture was concentrated by rotary evaporator at <30° C. The residue was purified by CombiFlash (2×330 g column, 0-30% EtOAc/heptanes) to give compound 7 (85% yields) as an orange oil/solid. The ¹H NMR analysis (C₆D₆) of the prepared material was shown in FIG. 12.

Compound 8

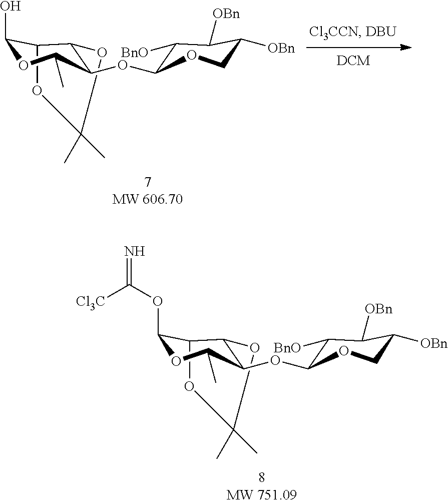

Compound 7 (33.5 g, 4.78 mmol) was dissolved in DCM (847 mL, 25 vol.) and charged to a 2 L 3-neck flask. The reaction mixture was cooled to 0-10° C. using an ice-water bath. DBU (10.7 mL, 1.3 equiv.) was added, followed by Cl₃CCN (63.6 mL, 11.5 equiv.) drop-wise. The reaction was then stirred at 0-10° C. After 1 hour the TLC analysis (TLC plates were pre-treated with 10% Et₃N/heptanes; eluent: heptanes:ethyl acetate 2:1 with 2% Et₃N, CAM stain) showed little starting material and compound 8 was observed (Rf 0.6). The reaction mixture was diluted with toluene (1000 mL, 30 vol) and washed with water (3×234 mL, 3×7 vol). The organic layer was dried over MgSO₄ and then filtered. The MgSO₄ was washed with toluene (167 mL, 5 vol). The filtrate was concentrated to dryness by rotary evaporator at <35° C.

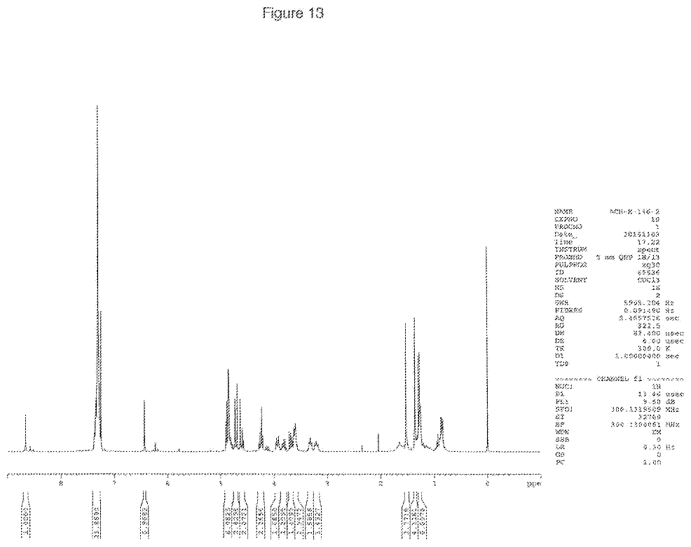

A mixture of heptanes/EtOAc/Et₃N (15:5:1) was prepared. The residue was dissolved with this solvent mixture and passed through a plug of silica gel (40 g) which was pre-treated with the solvent mixture. The plug was washed with this solvent mixture until all the desired product was eluted out. The desired fractions were concentrated at <30° C. and dried on high vacuum to give compound 8 as a yellow thick oil (39.3 g, 95% yields). The ¹H NMR analysis (CDCl₃) of the prepared material was shown in FIG. 13.

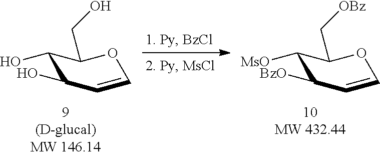

Compound 10

To a 2-L reactor was charged D-glucal (75.0 g, 0.51 mol, Chem-Impex), followed by pyridine (1125 mL, 15 vol). The resultant solution was cooled to 0-5° C. Benzoyl chloride (125 mL, 1.08 mol, 2.1 equiv.) was added slowly over 3 hours while maintaining the batch temperature at 0-5° C. The reaction was stirred at 0-5° C. for 1 hour and the TLC analysis (100% EtOAc and heptanes/EtOAc 3:1; CAM stain) showed that the starting material was completely consumed and some mono-benzoylated (Rf 0.6 in 100% EtOAc), di-benzoylated (Rf 0.20 in heptanes/EtOAc 3:1) and tri-benzoylated (Rf 0.35 in heptanes/EtOAc 3:1) were observed. Additional benzoyl chloride (12.0 mL, 0.2 equiv) was added over 15 minutes. The resultant reaction mixture was stirred for 1.5 hour at 0-5° C. and the TLC analysis showed the mono-benzoylated products were disappeared. MsCl (79.4 mL, 1.03 mol, 2.0 equiv) was then added at 0-5° C. over 1 hour. The reaction mixture was stirred at 0-5° C. for 20 minute and ambient temperature overnight. The TLC analysis (heptanes/EtOAc 3:1; CAM stain) showed that the di-benzoylated glucal (Rf 0.20) was completely consumed and the compound 10 was observed (Rf 0.16).

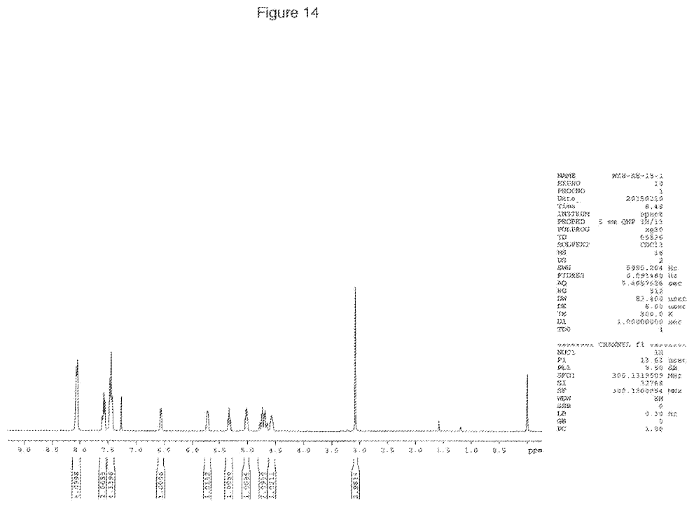

The reaction was quenched with methanol (90 mL, 1.2 vol) at <10° C. and diluted with MTBE (900 mL, 12 vol). The mixture was washed with water (900 mL, 12 vol) and then brine (200 mL, 2.7 vol). The combined aqueous layers were back-extracted with MTBE (2×150 mL, 2×2 vol). The organic layers were combined and concentrated to remove most of pyridine at <30° C. The residue (275 g) was dissolved in DCM (400 mL, 5.3 vol) and washed with water (3×100 mL, 3×1.3 vol). The organic layer was then concentrated to dryness and re-crystallized with MTBE (300 mL, 4 vol) to give the 1^{st} crop of compound 10 (116.1 g, 52.3% yield) as a pale yellow solid. The mother liquor was concentrated and the resultant residue (107 g) was further purified by chromatography (2×330 g column; 0-40% EtOAc in heptanes). The fractions containing desired product were concentrated and recrystallized with MTBE (100 mL, 1.3 vol) to give a second crop of compound 10 (31.5 g, 14.2% yield) as an off-white solid. The combined yields were 147.6 g (66.5% yields). The 1H NMR analysis (CDCl₃) of the prepared material was shown in FIG. 14.

Compound 11

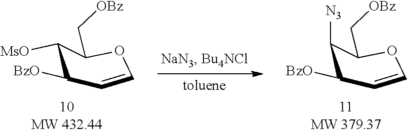

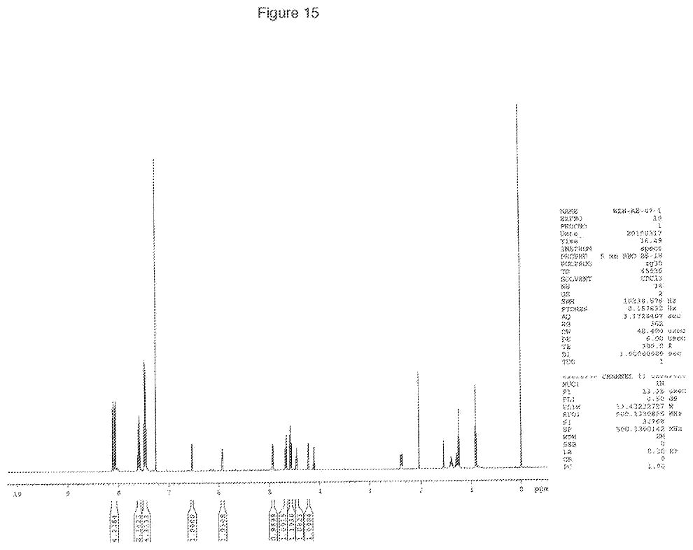

To a 1-L 3-neck flask was charged compound 10 (45.0 g), followed by toluene (350 mL, 7.8 vol). Tetrabutylammonium chloride (63.6 g, 229 mmol, 2.2 equiv) and sodium azide (25.0 g, 385 mmol, 3.7 equiv) were then added, followed by toluene (168 mL, 3.7 vol). The resultant mixture was then slowly heated to 105° C. and stirred for 18 hours at 100-110° C. The TLC analysis (heptanes/EtOAc 3:1; CAM stain) showed that only small amount of compound 10 (Rf 0.16) was present and compound 11 (Rf 0.46) was observed. The reaction mixture was cooled to ambient temperature and transferred to a separation funnel. The reaction flask was rinsed with toluene (450 mL, 10 vol) and water (450 mL, 10 vol) and the rinses were also transferred to the separation funnel. The organic layer was separated and washed with water (450 mL, 10 vol). The organic layer was concentrated at <30° C. and the residue was purified by CombiFlash (330 g column, 0-15% EtOAc/heptanes) to give compound 11 (23.8 g, 60.3% yields) as a pale yellow thick oil. The ¹H NMR analysis (CDCl₃) of the prepared material was shown in FIG. 15. This material contained an impurity which might be derived from tetrabutylammonium salt (¹H NMR) and easily purged in the next step.

Compound 11 (Alternate Scheme)

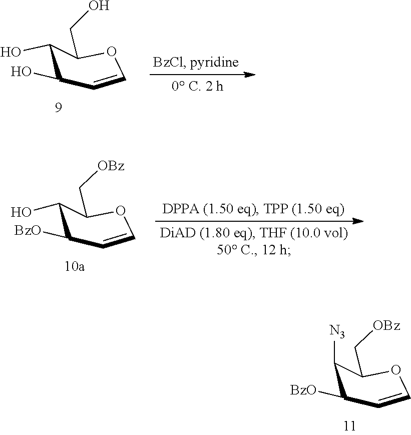

Compound 12

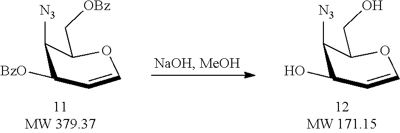

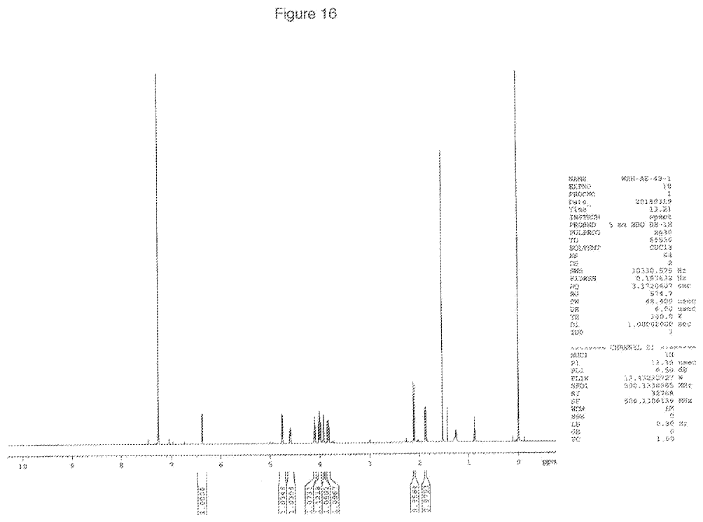

Compound 11 (45.3 g, 119 mmol, 1.0 equiv) was dissolved in methanol (544 mL, 12 vol) and charged to a 1-L 3-neck flask. To this mixture was added a NaOH solution (50 mg/mL in methanol, 33.4 mL, 41.8 mmol, 0.35 equiv) dropwise at ambient temperature. After addition the resultant mixture was stirred at ambient temperature. After 3.5 hours the TLC analysis showed the compound 11 was completely consumed (Rf 0.46, heptanes/EtOAc 3:1; CAM stain) and compound 12 was observed (Rf 0.65, 100% EtOAc; CAM stain). The reaction mixture was concentrated at <30° C. and the residue was purified by CombiFlash (220 g column, 30-100% EtOAc/heptanes) to give compound 12 (13.1 g, 64.2% yields) as a white solid. The ¹H NMR analysis (CDCl₃) of the prepared material was shown in FIG. 16.

Compound 13

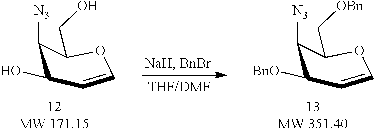

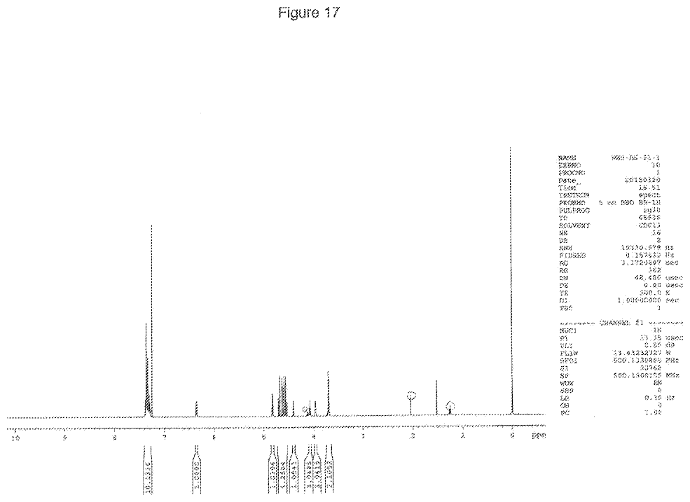

Compound 12 (13.1 g, 76.5 mmol, 1.0 equiv) was dissolved in THF (200 mL, 15 vol) and DMF (200 mL, 15 vol). The resultant mixture was charged to a 1-L 3-neck flask and cooled to 0-5° C. NaH (9.18 g, 60% dispersion in oil, 230 mmol, 3.0 equiv) was added portionwise over 10 minutes at 0-5° C. The mixture was stirred at 0-10° C. for 30 minutes before benzyl bromide (36.4 mL, 306 mmol, 4.0 equiv) was charged slowly over 20 minutes while maintaining the batch temperature below 10° C. The reaction was warmed to ambient temperature and stirred overnight. The TLC analysis showed the compound 12 was completely consumed (Rf 0.65, 100% EtOAc; CAM stain) and compound 13 was observed (Rf 0.19, 9:1 heptanes/EtOAc; CAM stain). The reaction mixture was cooled to 0-10° C. and methanol (9.0 mL, 0.7 vol) was added slowly with batch temperature below 10° C., followed by water (262 mL, 20 vol) at <10° C. The mixture was warmed to ambient temperature and extracted with EtOAc (2×200 mL, 2×15 vol). The combined organic layers were washed with saturated NaCl solution (1×50 mL, 1×4 vol) and concentrated at <30° C. The residue was purified by CombiFlash (330 g column, 0-15% EtOAc/heptanes) to give compound 13 (24.0 g, 89.1% yields) as a pale yellow oil. The ¹H NMR analysis (CDCl₃) of the prepared material was shown in FIG. 17.

Compound 14

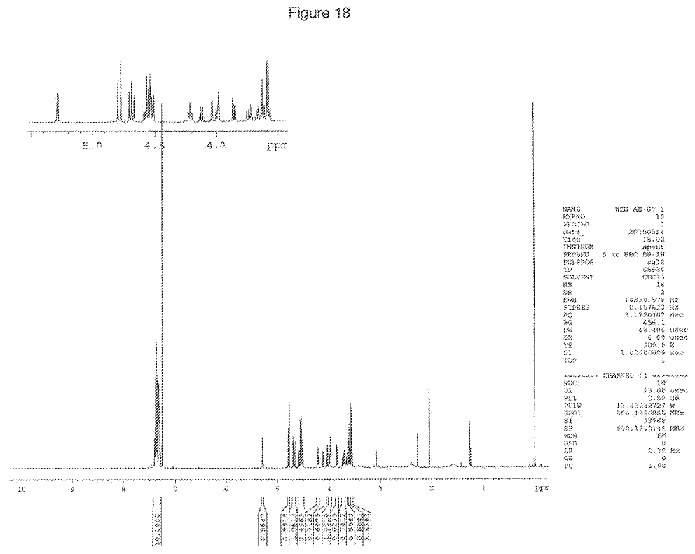

Compound 13 (13.8 g, 39.3 mmol, 1.0 equiv.) was dissolved in THF (242 mL, 17.5 vol) and transferred to a 1-L 3-neck flask. Tert-butanol (104 mL, 7.5 vol) and water (35 mL, 2.5 vol) were then added. The OsO₄ solution (13.8 mL, 2.5 wt % in t-butanol) was added in one portion to afford a pale yellow solution. After stirring at ambient temperature for 30 minutes NMO solution (6.9 mL, 50% in water) was added. After 3.5 hours further NMO (6.9 mL, 50% in water) was added. After another 3 hours another portion of NMO solution (6.9 mL, 50% in water) was added and the mixture was stirred at ambient temperature for 17 hours. The last portion of NMO solution (6.9 mL, 50% in water) was added and stirring continued for 5 hours. The TLC analysis (heptanes/EtOAc, 1:1; CAM stain; starting material Rf 0.8 and product Rf 0.3) showed only trace amount of starting material. An aqueous Na₂SO₃ solution (55.2 g Na₂SO₃ in 276 mL H₂O) was added slowly and the resultant mixture was stirred at ambient temperature for 30 minutes. The mixture was diluted with water (138 mL, 10 vol) and extracted with EtOAc (276 mL, 20 vol). The organic layer was dried over MgSO4, filtered, and concentrated to give compound 14 (15.3 g, 100% yields) as a pale brown thick oil, which was used directly in the next step without further purification. The ¹H NMR analysis (CDCl₃) of the prepared material was shown in FIG. 18.

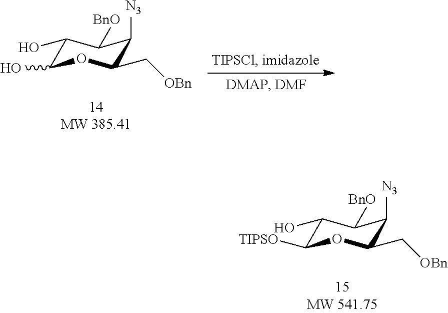

Compound 15

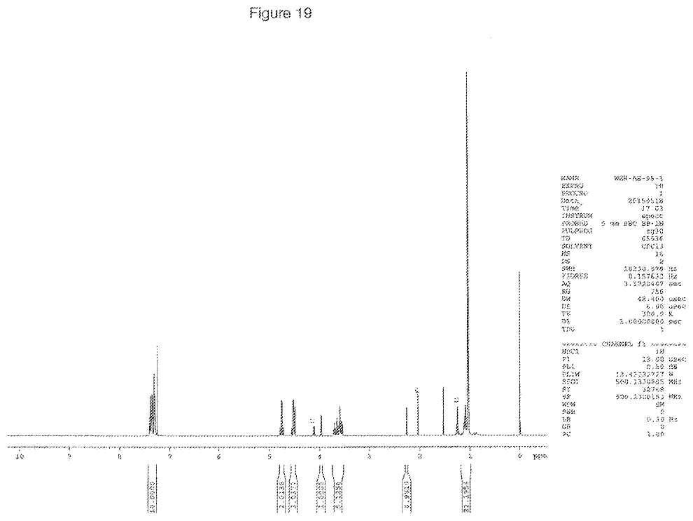

Compound 14 (15.3 g, 39.7 mmol, 1.0 equiv) was dissolved in DMF (80 mL, 5.2 vol). Imidazole (6.49 g, 95.3 mmol, 2.4 equiv) was added followed by DMAP (0.49 g, 4.0 mmol, 0.1 equiv). The mixture was cooled to 0-10° C. with water/ice bath and TIPSCl (12.7 mL, 59.6 mmol, 1.5 equiv) was added dropwise. The water/ice bath was removed and the reaction was stirred at ambient temperature for 17 hours. The TLC analysis (heptanes/EtOAc 1:1; CAM stain; starting material Rf 0.2) showed a full conversion and compound 15 was observed (heptanes/EtOAc 4:1; CAM stain; product Rf 0.4). The mixture was cooled to 0-10° C. and water (306 mL, 20 vol) was added slowly while maintaining the batch temperature at <20° C. The mixture was warmed to ambient temperature and extracted with EtOAc (306 mL, 20 vol; then 77 mL, 5 vol). The combined organic layer was washed with water (2×306 mL, 2×20 vol) and 20% brine (77 mL, 5 vol) and concentrated at <30° C. The residue was purified by CombiFlash (330 g column, 0-10% EtOAc/heptanes) to give compound 15 (15.2 g, 70.4% yields) as a thick oil. The ¹H NMR analysis (CDCl₃) of the prepared material was shown in FIG. 19.

Compound 16

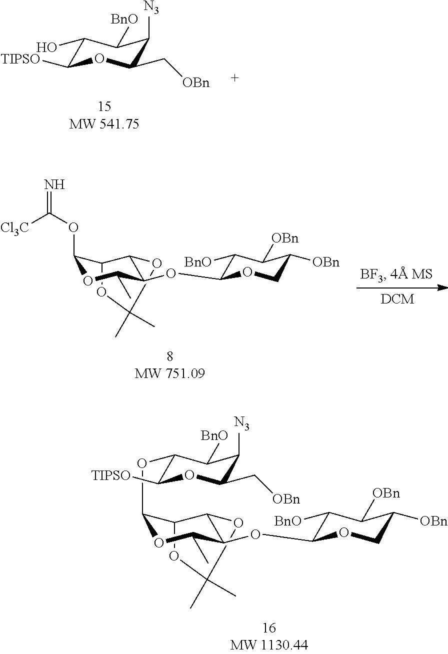

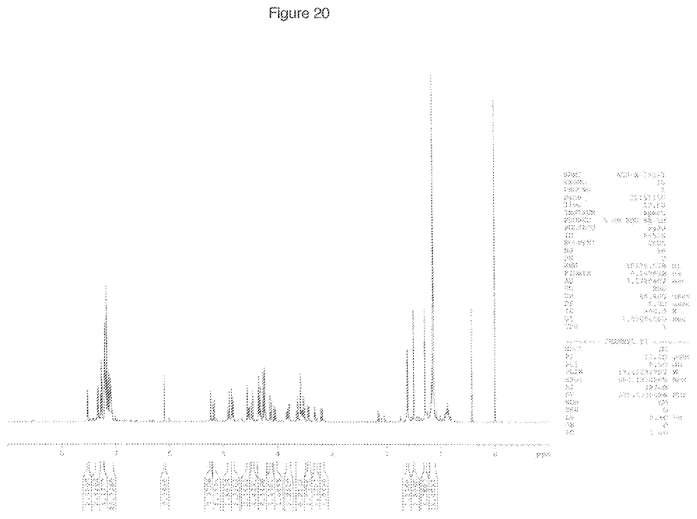

Compound 15 (12.8 g, 23.6 mmol, 1.0 equiv) and compound 8 (19.5 g, 26.0 mmol, 1.1 equiv) were co-evaporated with toluene (2×100 mL) at <30° C. and dissolved in DCM (320 mL, 25 vol). A powder of 4 Å molecular sieve (12.8 g, 1 wt) was added. The resultant reaction mixture was stirred at ambient temperature for 30 minutes and cooled to −45 to −35° C. using a dry ice/acetone bath. BF₃.OEt₂ (0.58 mL, 4.7 mmol, 0.2 equiv) was added and the reaction was stirred at −45 to −35° C. After 60 minutes an additional compound 8 (3.6 g, 4.7 mmol, 0.2 equiv) in DCM (38 mL) was added at −45 to −35° C. After another 1 hour the TLC analysis (3:1 heptanes/EtOAc, CAM stain) showed compound 16 was observed (Rf 0.5). The reaction mixture was quenched with TEA (12.8 mL) at <−40° C. and warmed to ambient temperature. The mixture was concentrated to dryness and the residue was purified by CombiFlash (330 g column, 0-10% EtOAc/heptanes) to give compound 16 (12.7 g, 48% yield) as a thick oil. The ¹H NMR analysis (CDCl₃) of the prepared material was shown in FIG. 20.

Compound 17

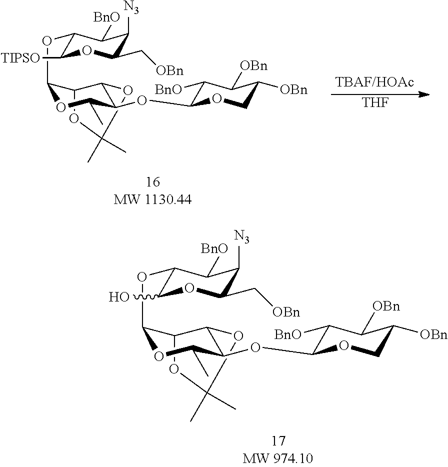

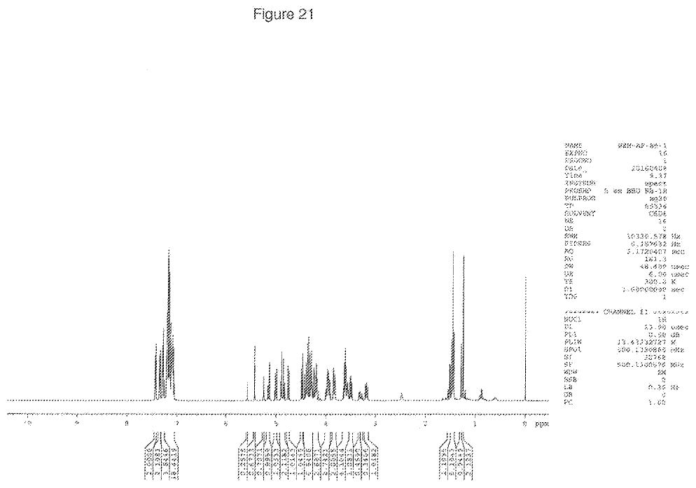

Compound 16 (99.8 g, 88.3 mmol, 1.0 equiv) was dissolved in THF (1.5 L, 15 vol) and transferred to 3-L 3-neck flask. A mixture containing TBAF (105.9 mL, 105.9 mmol, 1.2 equiv; 1.0 M solution in THF), acetic acid (2.5 mL, 44.1 mmol, 0.5 equiv), and THF (35 mL) was added slowly over 40 minutes via an addition funnel. The addition funnel was rinsed with THF (20 mL). After overnight reaction at ambient temperature the TLC analysis (3:1 heptanes/EtOAc, CAM stain) showed a small amount of compound 16. Acetic acid (7.0 mL) and methanol (100 mL) were then added. The resultant mixture was stirred at ambient temperature for 30 minutes and concentrated at <30° C. The crude (144 g) was purified by chromatography (1.0 kg silica gel; 0-30% EtOAc/heptanes) to give compound 17 (67.8 g, 79% yield) as a pale yellow foam/thick oil. The ¹H NMR analysis (C₆D₆) of the prepared material was shown in FIG. 21.

Compound 18

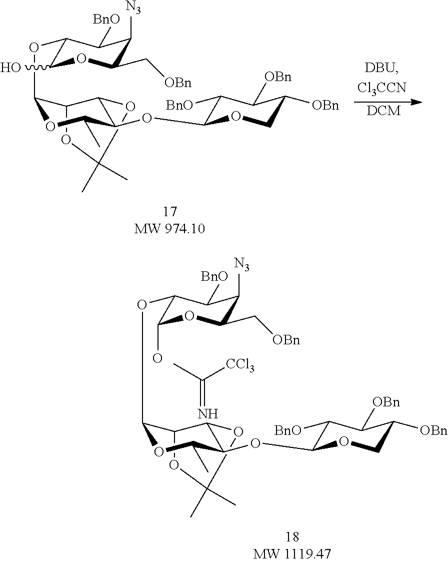

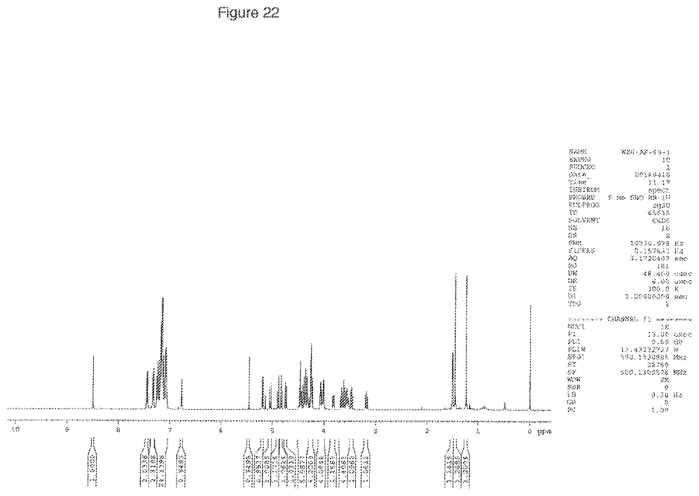

Compound 17 (67.7 g, 69.6 mmol, 1.0 equiv) was dissolved in DCM (1356 mL, 20 vol) and transferred to a 2-L 3-neck flask. The reaction mixture was cooled to 0-10° C. DBU (13.5 mL, 90.5 mmol, 1.3 equiv.) was charged, followed by Cl₃CCN (80.3 mL, 800.4 mmol, 11.5 equiv.) dropwise at 0-10° C. After 4.5 hours at 0-10° C. the TLC analysis (1:2 EtOAc/heptane with 2% TEA, CAM stain) showed trace amount of compound 17 (Rf 0.5) and the compound 18 was observed (Rf 0.7). The reaction was diluted with toluene (2030 mL, 30 vol) and washed with aqueous NaCl solution (2×, each wash contained 406 mL (6 vol) of water and 136 mL (2 vol) of saturated NaCl solution) and then saturated NaCl solution (406 mL, 6 vol). The organic layer was then dried over MgSO4 (68 g, 1 wt), filtered, washed with toluene (340 mL, 5 vol), and concentrated at <30° C. The residue (94.6 g) was dissolved in a mixture of heptanes/EtOAc/TEA (15:5:1) and filtered through a plug of silica gel (900 g, pre-treated with 5% Et₃N in heptanes) and washed with a mixture of heptanes/EtOAc/TEA (15:5:1) until all the products were eluded out. The desired fractions were concentrated at <30° C. and dried on high vacuum to give compound 18 as a yellow foam/thick oil (67.5 g, 87% yields). The ¹H NMR analysis (C₆D₆) of the prepared material was shown in FIG. 22.

Compound 19

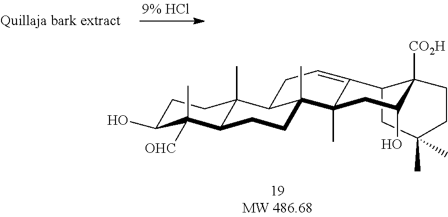

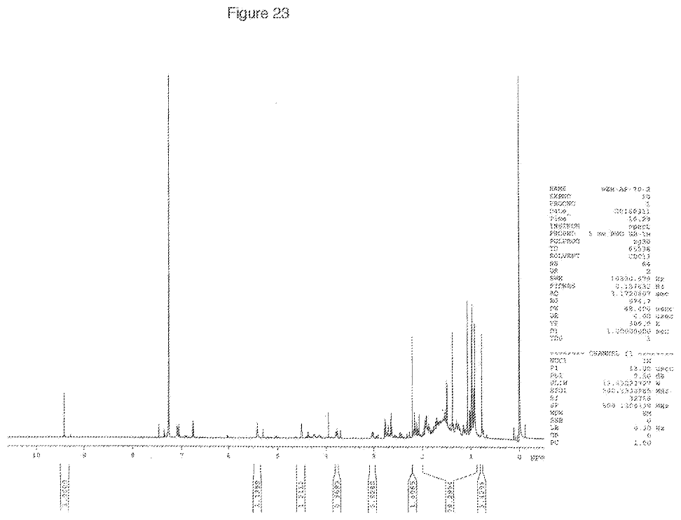

To a 3-neck flask was charged quillaja bark extract (500 g, 1 wt), followed by 9% aq. HCl (5 L, 10 vol). The resultant mixture was heated to 88-92° C. and stirred for 4 hours. The resultant brownish mixture was cooled to ambient temperature. The reaction was diluted with EtOAc (5.0 L, 10 vol) and stirred at ambient temperature for 10 minutes. The mixture was filtered through a pad of Celite (250 g, 0.5 wt) and washed with EtOAc (2.5 L, 5 vol). The filtrate was transferred to a cylindrical reactor and stirred at ambient temperature for 15 minutes. The agitation was stopped and the mixture was allowed to settle for a minimum of 15 minutes. The organic layer was separated and the aqueous layer was extracted with EtOAc (2.5 L, 5 vol). The organic layers were combined and concentrated to dryness. The residue (269 g) was purified by silica gel chromatography (1000 g silica gel, 0-40% EtOAc/heptanes) to give compound 19 (31.3 g, 6.3 wt % yields) as a yellow solid. The ¹H NMR analysis (CDCl₃) of the prepared material was shown in FIG. 23. A mixture fraction (86.8 g) was also obtained and combined with other mixed fractions for further chromatography purification.

Alternative Route to Compound 19

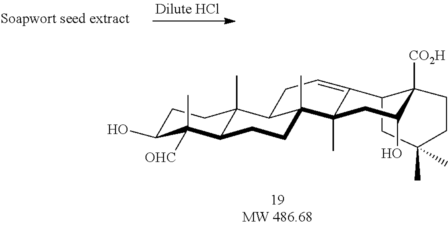

Charge a 3-neck flask with Soapwort seed extract extract, followed by dilute aq. HCl (5 L, 10 vol). Stir the resultant mixture for an optimum time period with possible heating. Cool the resultant mixture to ambient temperature. Dilute reaction materials in an organic layer and stir. Filter through a pad of Celite and wash with more organic solvent. Separate organic layer from aqueous layer and wash aqueous layer repeated times with additional organic solvent. Combine organic layers and remove organic solvent. Purify residue by silica gel chromatography to give compound 19 as a solid.

Compound 20

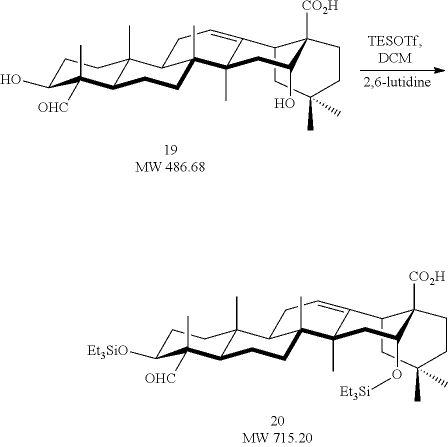

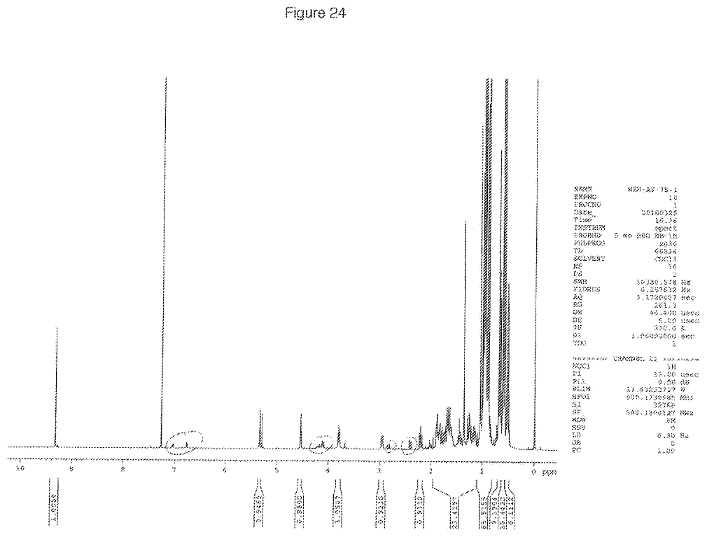

Compound 19 (57.4 g, 118 mmol) was charged to a 2-L 3-neck flask with the help of DCM (1148 mL, 20 vol) and 2,6-lutidine (112.6 mL, 8.2 equiv). A brown solution was obtained. The reaction was cooled to 0-5° C. TESOTf (106.7 mL, 472 mmol, 4.0 equiv) was then added dropwise at <10° C. via an addition funnel. The addition funnel was rinsed with DCM (20 mL, 0.35 vol) and charged to the reaction. The reaction was stirred at 0-10° C. for 3.5 hours and TLC (1:1 heptanes/EtOAc; CAM stain) showed all the starting material was consumed. The mixture was diluted with EtOAc (1148 mL, 20 vol) and washed with 0.5 M HCl (1148 mL, 20 vol). The organic layer was washed with a mixture containing sat. NaHCO₃ solution (574 mL, 10 vol) and sat. NaCl solution (385 mL, 6.7 vol). The aqueous layer was back-extracted with EtOAc twice (574 mL, 10 vol; then 287 mL, 5 vol). The combined organic layers were concentrated to dryness <30° C. The residue (143 g) was purified by silica gel chromatography (900 g silica gel, 0-15% EtOAc/heptanes) to give compound 20 (51.2 g, 61% yields) as an orange thick oil (containing some silicon impurities and other small impurities). The 1H NMR analysis (CDCl₃) of the prepared material was shown in FIG. 24. A mixed fraction (10.8 g) was also obtained and combined with other mixed fractions for further chromatography purification.

Compound 21

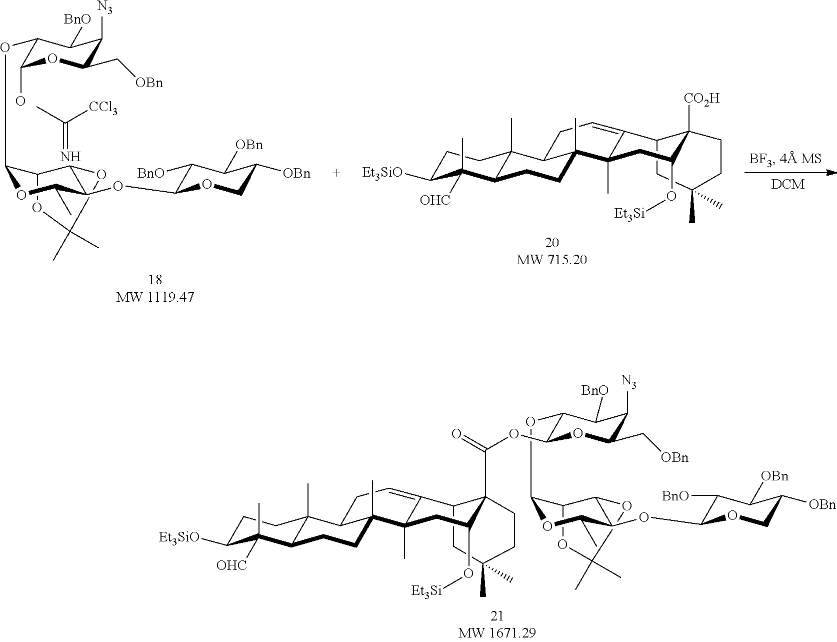

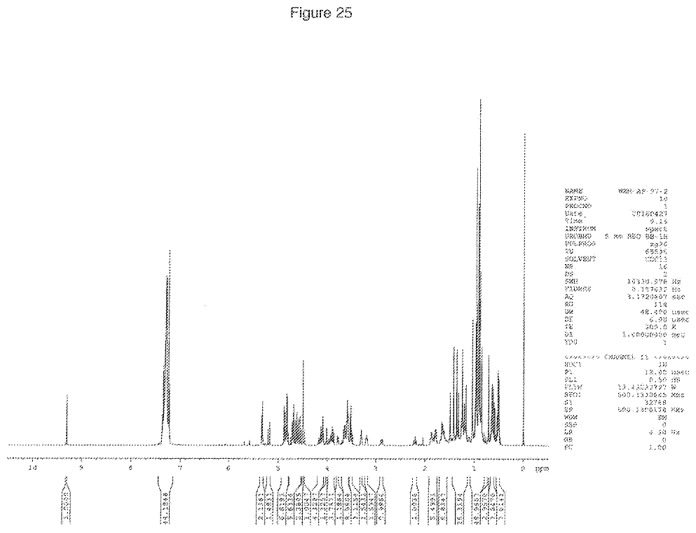

Compound 18 (52.0 g, 46.4 mmol, 1.0 equiv) and pure compound 20 (36.5 g, 51.1 mmol, 1.1 equiv) were charged to a 3-L 3-neck flask with the help of anhydrous DCM (1820 mL, 35 vol). 4 Å molecular sieve powder (78.0 g, 1.5 wt) was added and the resultant mixture was stirred at ambient temperature for 50 minutes. The reaction was cooled to −35±5° C. and BF₃.OEt₂ (1.15 mL, 9.3 mmol, 0.2 equiv) was added dropwise at −35±5° C. After 4 hours at −35±5° C. TEA (52 mL, 1 vol) was then added and the mixture was stirred at −30° C. for 20 minutes and ambient temperature for 1 hour. The mixture was concentrated to dryness at <25° C. to give crude compound 21 (182.3 g). A synthesis of compound 21 was performed on a 15 g scale under similar conditions to give crude compound 21 (51.6 g). The aforementioned two lots of crude compound 21 (182.3 g; 51.6 g) were combined and purified by silica gel chromatography (1.2 kg silica gel, 0-20% EtOAc/heptanes+1% TEA) to give compound 21 (85.0 g, 85% yield based on 67.0 g of compound 18 input) as a yellow foam/thick oil. The ¹H NMR analysis (CDCl₃) of the prepared material was shown in FIG. 25. The TLC analysis showed the material contained an impurity which will be purged in the next step.

Compound 22

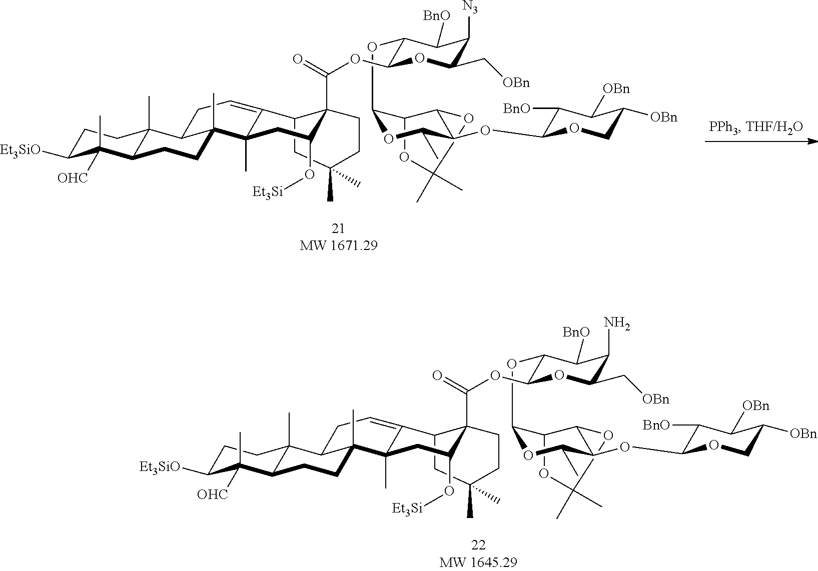

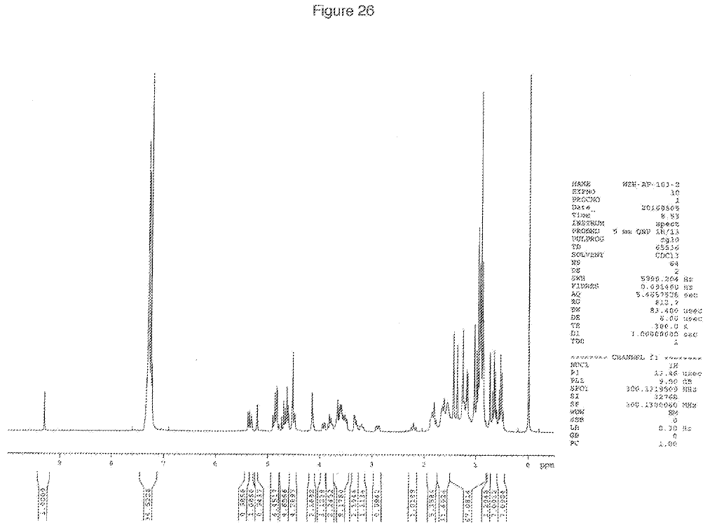

Compound 21 (84.5 g, 50.6 mmol, 1.0 equiv) was dissolved in THF (1268 mL, 15 vol) and transferred to a 3-L 3-neck flask. Triphenylphosphine (79.6 g, 303.4 mmol, 6.0 equiv) was added. The resultant solution was heated slowly to 40-45° C. After 18 hours water (338 mL, 4 vol) and THF (507 mL, 6 vol) were added. The reaction was heated to 55-60° C. and stirred for 28 hours. The reaction was cooled to ambient temperature and concentrated to dryness. The residue was co-evaporated subsequently with toluene (2×200 mL), anhydrous THF (8×200 mL), and EtOAc (1×200 mL) to remove the remaining water. The residue (177 g) was purified by silica gel chromatography (1.0 kg silica gel, 0-40% EtOAc/heptanes) to give compound 22 (54.4 g, 65% yield) as a white foam/thick oil. The ¹H NMR analysis (CDCl₃) of the prepared material was shown in FIG. 26.

Compound 23

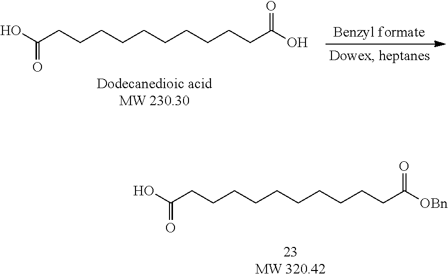 [SMILES: O=C(O)CCCCCCCCCCC(=O)O ; O=C(O)CCCCCCCCCCC(=O)OCc1ccccc1]

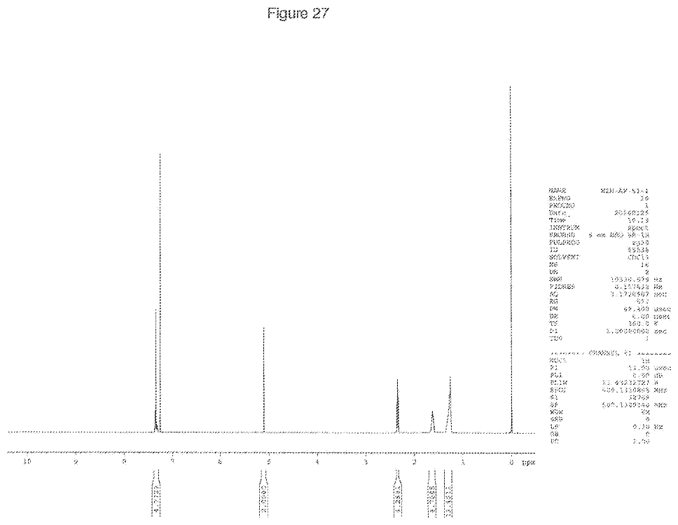

To a 3-L 3-neck flask was charged dodecanedioic acid (150.0 g, 1 wt), followed by heptanes (1350 mL, 9 vol) and benzyl formate (315 mL, 2.1 vol) to afford a white slurry. Dowex 50WX4 resin (210 g, 1.4 wt, hydrogen form, 50-100 mesh) was then added. Rinse the resin container with heptanes (150 mL, 1 vol) and charge to the reaction. The mixture was heated to 80° C. and stirred for 24 hours. The mixture was cooled to ambient temperature. The agitation was stopped and the reaction was settled down for 30 minutes. The mixture was decanted into a filter and filtered. The remaining solids in the reactor was added DCM (450 mL, 3 vol) and stirred for 30 minutes. The mixture was filtered using the same filter and washed the resin with DCM (2×300 mL, 2×2 vol). The filtrate was concentrated to give an off-white residue (389 g). The residue was stirred with heptanes (1.5 L, 10 vol) at ambient temperature to afford a white slurry. The mixture was filtered and washed with heptanes (2×200 mL, 2×1.3 vol) to give the 1^{st} crop of compound 23 (73.0 g) as a white solid. The filtrate was concentrated to give a pale yellow oil (311 g), which was purified by chromatography purification (800 g silica gel; 100% heptanes, then 1:1 DCM/heptanes then 45:45:10 DCM/heptanes/EtOAc). The fractions containing compound 23 and small amounts of impurities were combined and concentrated to give a white residue (54.3 g). The residue was stirred with heptanes (200 mL) for 3 hours, filtered, and washed with heptanes (2×50 mL) to give the 2^{nd} crop of compound 23 (40.6) as a white solid. The 1^{st} crop and 2^{nd} crop of compound 23 were combined and stirred with heptanes (455 mL) for 1 hour. The mixture was filtered and washed with heptanes (2×110 mL) to give compound 23 (111.8 g, 54% yield) as a white solid. The ¹H NMR analysis (CDCl₃) of the prepared material was shown in FIG. 27.

Compound 24

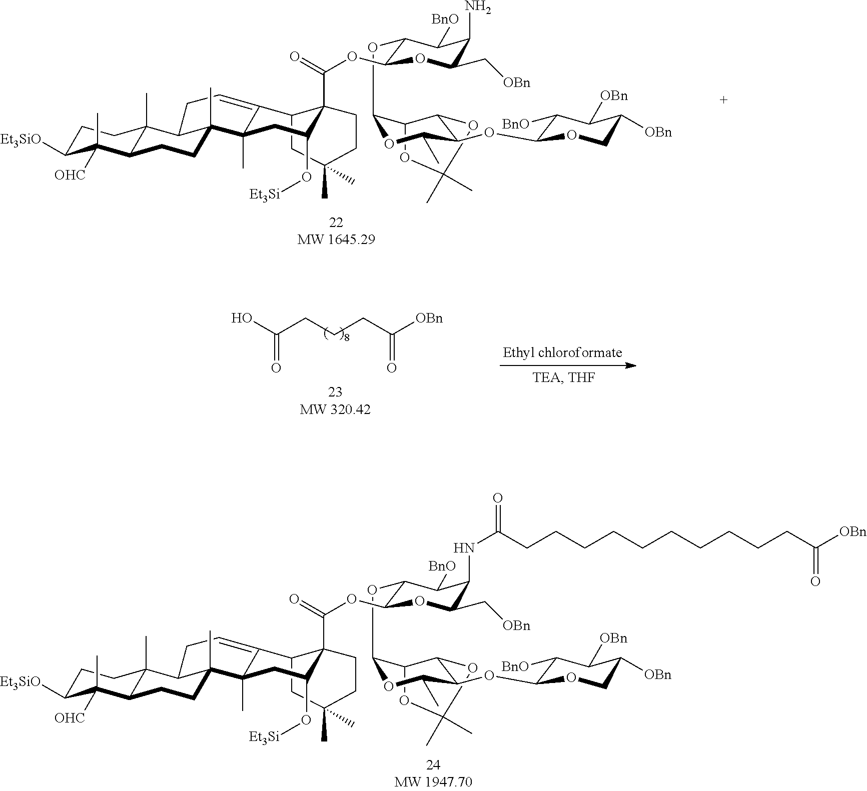

Flask 1: Compound 23 (26.4 g, 82.4 mmol, 2.5 equiv) was charged to a 1-L 3-neck flask, followed by THF (528 mL, 20 vol). The reaction mixture was cooled to 0-10° C. TEA (21.8 mL, 156.5 mmol, 4.75 equiv) was added. Ethyl chloroformate (6.5 mL, 68.5 mmol, 2.08 equiv) was then added dropwise while maintaining the batch temperature <10° C. The resultant white slurry was stirred at 0-10° C. for 30 minutes and at ambient temperature for 3-4 hours.

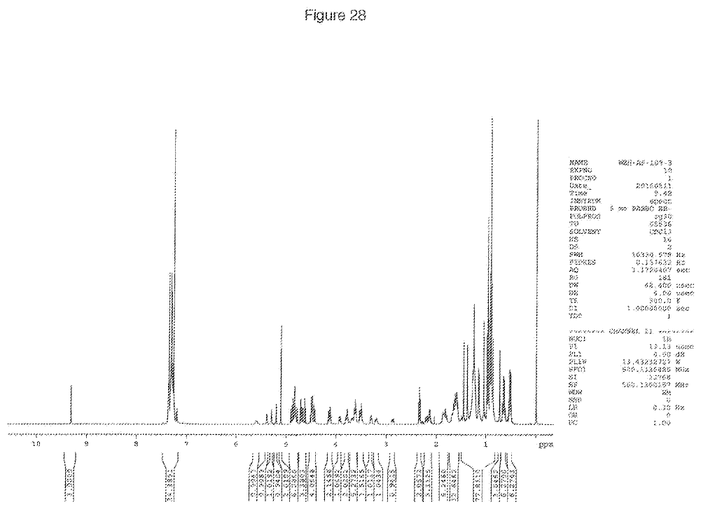

Flask 2: Compound 22 (54.2 g, 32.9 mmol, 1.0 equiv) was transferred to a 3-L 3-neck flask with the help of THF (1084 mL, 20 vol). The resultant solution was cooled to 0-10° C. The contents in Flask 1 were slowly transferred to Flask 2 via a cannula while maintaining the batch temperature in Flask 2<6° C. The Flask 1 was rinsed with THF (50 mL, 1 vol) and the rinse was also charged to Flask 2. The reaction mixture in Flask 2 was stirred overnight while slowly warming to ambient temperature. Methanol (108 mL, 2 vol) was then added and the resultant mixture was stirred at ambient temperature for 1 hour. The reaction mixture was concentrated to dryness. The residue (96 g) was purified by silica gel chromatography (1.2 kg silica gel, 0-20% EtOAc/heptanes+2% TEA) to give 1st crop of compound 24 (43.8 g) as a pale yellow thick oil. A mixed fraction (16.1 g) was further purified by chromatography (330 g silica gel, 0-20% EtOAc/heptanes+2% TEA) to give 2^{nd} crop of compound 24 (9.4 g). Two crops of compound 24 were combined go give compound 24 (53.9 g, 84.0% yields) as a white foam/thick oil. The ¹H NMR analysis (CDCl₃) of the prepared material was shown in FIG. 28.

Compound 25

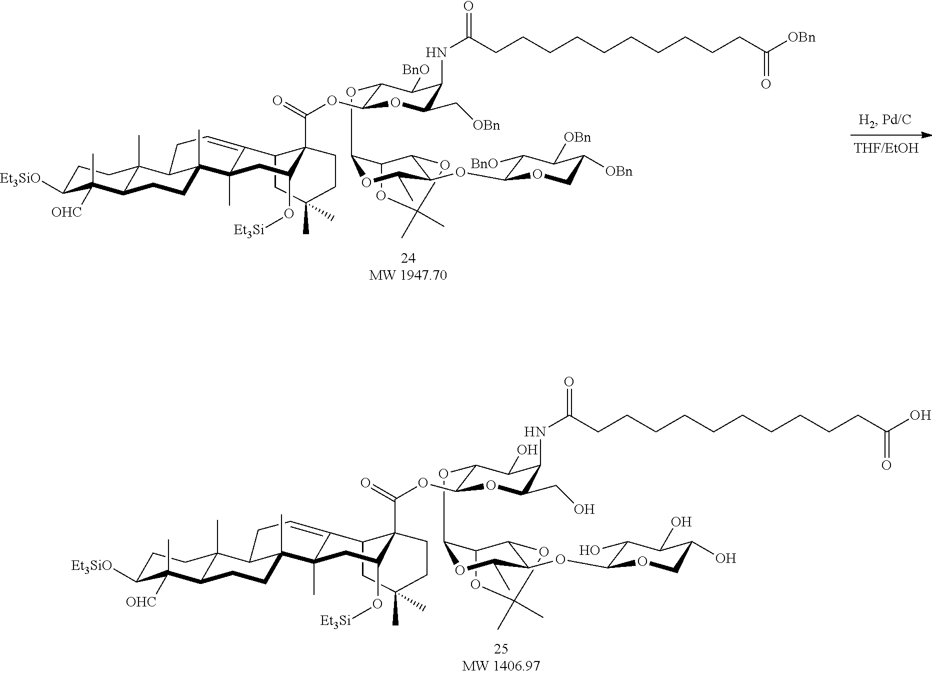

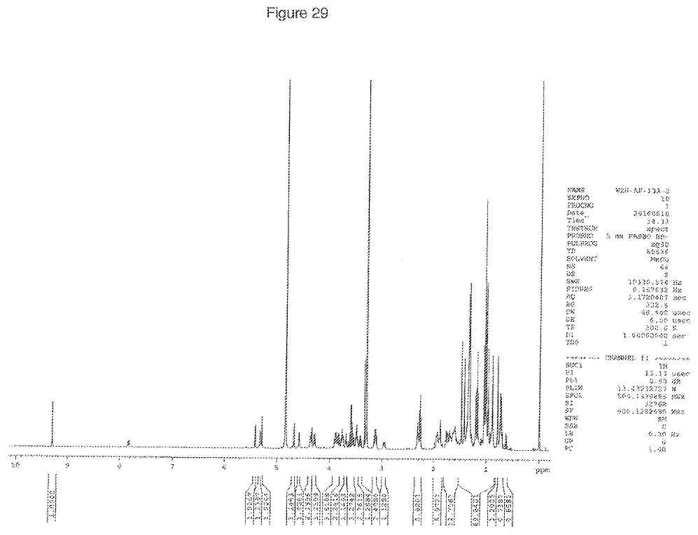

Compound 24 (38.7 g) was transferred to a 2-L hydrogenation reactor with help of THF (387 mL, 10 vol). Pd/C (38.7 g, 10 wt % Pd on dry basis, 50% water, 1.0 wt) was added, followed by ethanol (387 mL, 10 vol). The mixture was stirred overnight at ambient temperature under 45-50 psi of H2. The batch was then filtered through a Celite pad (116 g, 3 wt) and washed with EtOH (2×194 mL, 2×5 vol; then 2×310 mL, 2×8 vol). The combined filtrate was filtered through filter paper and concentrated to give compound 25 (23.3 g, 83% yields) as an off-white solid. The ¹H NMR analysis (CD₃OD) of the prepared material was shown in FIG. 29. This material was used in the next step without further purification.

Compound 26 (TQL-1055)

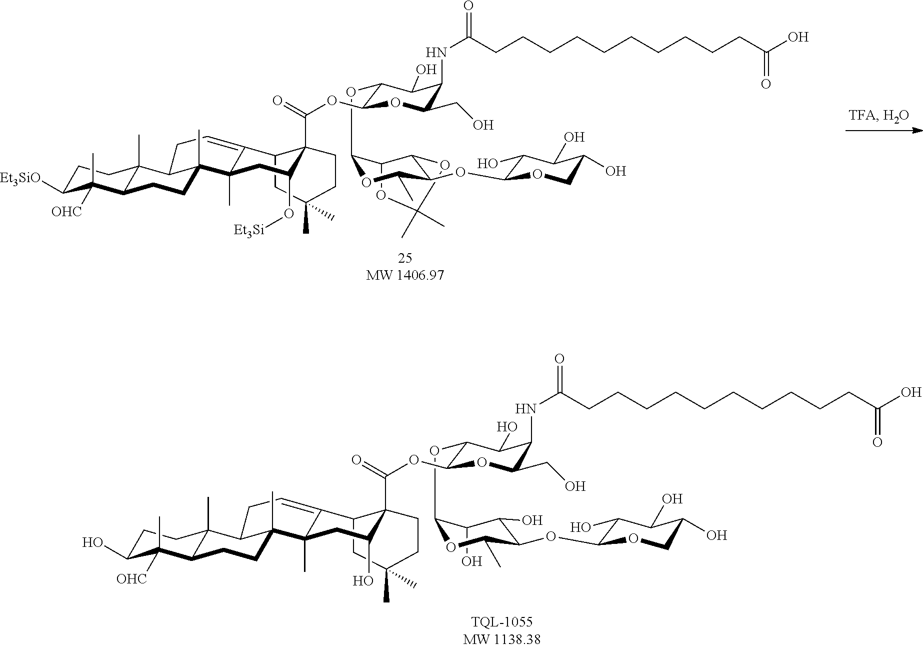

Compound 25 (50.7 g) in a mixture of trifluoroacetic acid (TFA, 811 mL, 16 vol) and water (203 mL, 4 vol) was stirred at 0-10° C. for 3.5 hours. The mixture was then co-evaporated with toluene via rotatory vaporation until all the TFA and water were removed. The residue was dissolved in MeOH (350 mL) and concentrated to give an off-white solid (52.5 g). The solids were purified by chromatography (2.2 kg silica gel, 0-25% DCM/MeOH) to give compound 26 (15.9 g). The mixed fractions were re-purified by chromatography (1.1 kg silica gel, 0-25% DCM/MeOH) to give another crop of compound 26 (8.7 g).

The aforementioned two lots of compound 26 (15.9 g; 8.7 g) were combined with two other lots of compound 26 (7.0 g; 10.6 g) to generate a single lot of crude compound 26 (42.2 g). Compound 26 (10.0 g) was then purified again by chromatography (600 g silica gel, 0-25% DCM/MeOH) to give compound 26 (5.5 g). This material was then rinsed with 60/40 MeOH/water 6 times (each time 1 L of solvents mixture). The mixture was filtered and dried to afford pure compound 26 (3.5 g). The HPLC analysis showed 96.4% AUC purity.

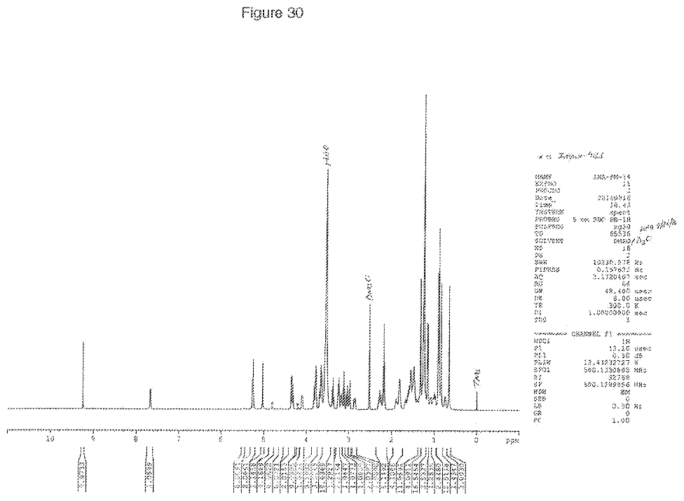

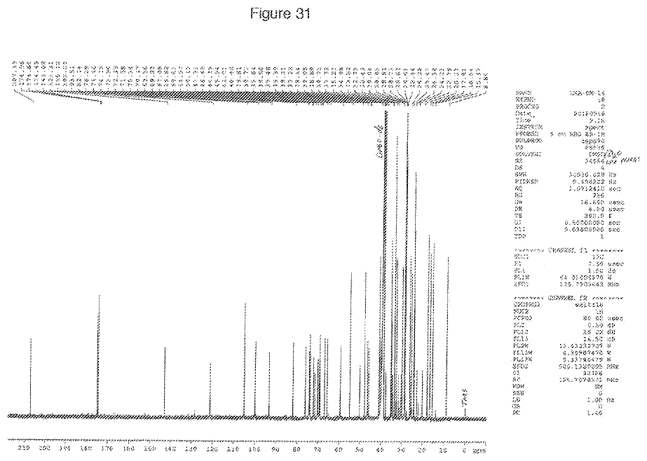

The four lots of purified material were then dissolved in MeOH and combined. The mixture was diluted with water and concentrated to remove MeOH under vacuum. The resulting mixture was then lyophilized to give compound 26 (20.2 g) as a white fluffy solid. The ¹H and ¹³C NMR are shown in FIG. 30-31.

### Example 2: Synthesis of SQS-21 (Api and Xyl)

It would be understood by one of ordinary skill in the art that common reaction intermediates shown in Examples 1 and 2, and/or protected or modified versions thereof, can be produced according to the schemes shown in either example. Additionally, it is within the level of ordinary skill in the art to modify or adapt the reactions shown in Examples 1 and 2 in order to produce compounds encompassing Formula I or Formula II as described in the present application.

Isolation and Selective Protection of Branched Trisaccharide-Triterpene Prosapogenin:

Part A: Isolation of Branched Trisaccharide-Triterpene Prosapogenins from Quil A.

1. In a 250-mL round-bottomed flask equipped with a reflux condenser, Quil A (1.15 g) and potassium hydroxide (0.97 g, 17 mmol) are suspended in EtOH/water (1:1) (50 mL), then the mixture is heated to 80° C. for 7 h.

2. The reaction is cooled to 0° C., neutralized with 1.0 N HCl, and concentrated to approximately one-half volume (care must be taken to avoid excessive foaming and bumping; water bath should be kept at 35° C. and pressure decreased slowly).

3. The mixture is frozen and lyophilized, and the resulting dry solid is purified by silica gel chromatography (CHCl₃/MeOH/water/AcOH, 15:9:2:1). The major product corresponding to the main spot observed by TLC is isolated by concentrating the desired fractions.

4. The resulting solid is dried by azeotropic removal of solvents with toluene (2×20 mL) and lyophilized in MeCN/water (1:1) (3×15 mL) to provide a mixture of prosapogenins (5:6, 2.5:1) as a light tan foam (˜0.55 g, 50% mass yield). These xylose- and rhamnose-containing prosapogenins correspond to the two most abundant trisaccharide-triterpene fragments found in QS saponins, and are advanced to the next protection step without further purification.

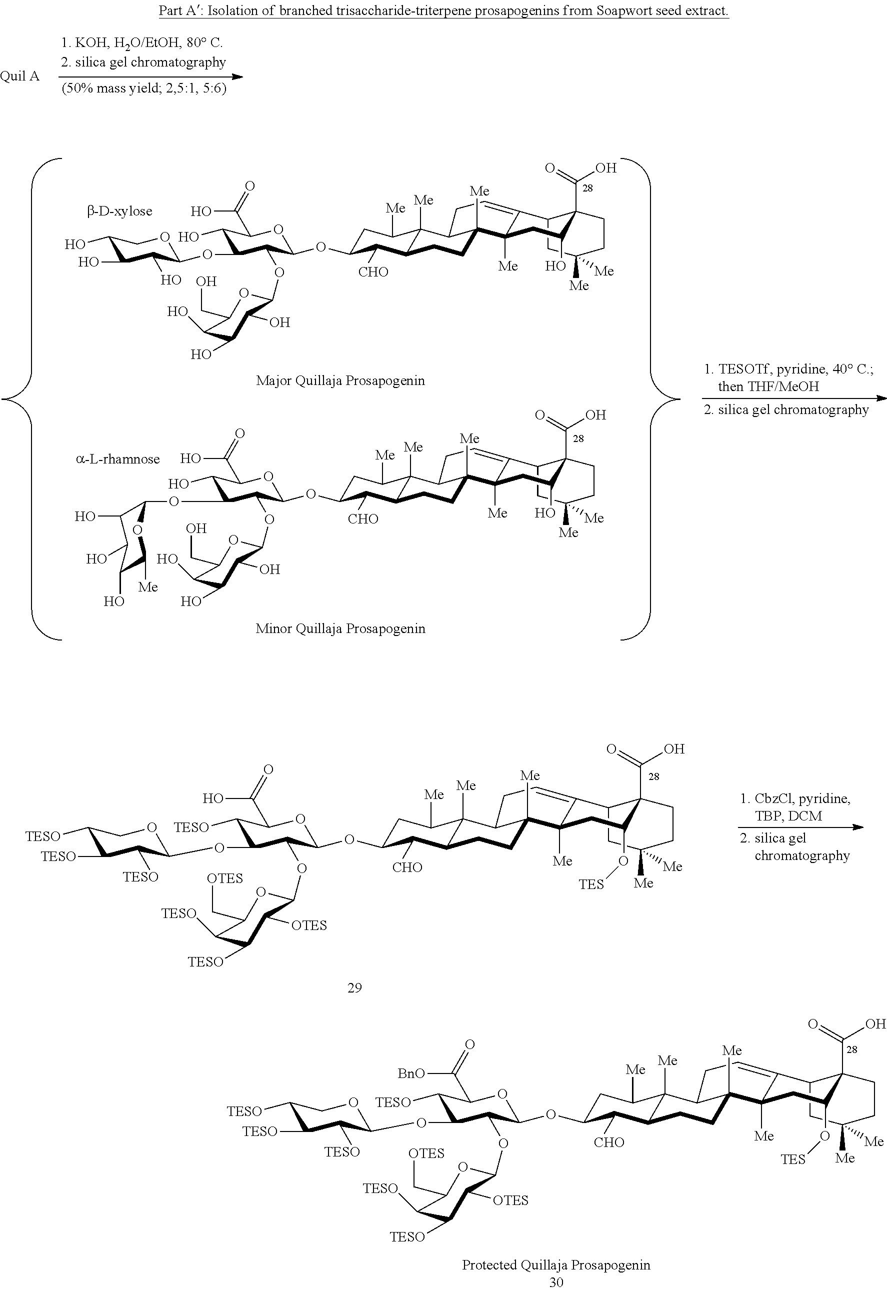

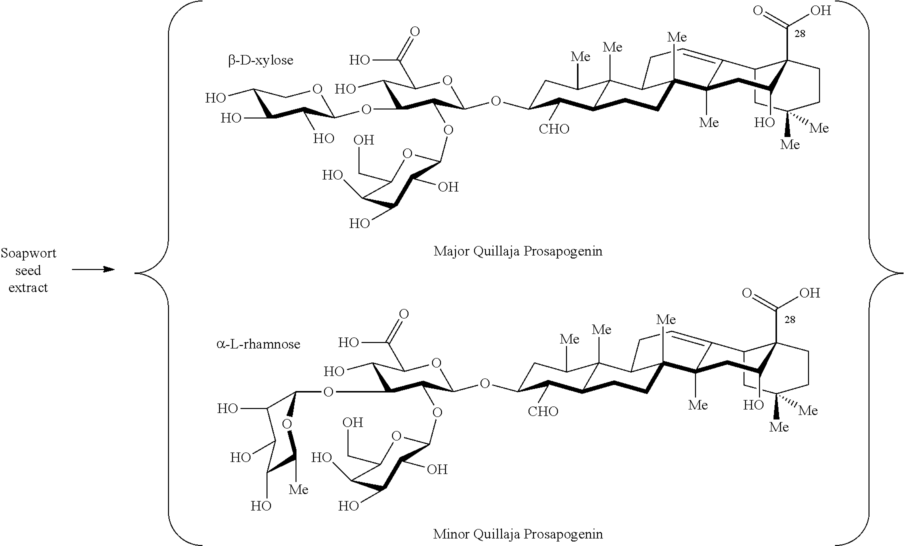

Isolation of the branched trisaccharide from Soapwort seed extract may proceed in a substantially similar fashion to the procedure laid out above.

Part B: Synthesis of Triethylsilyl (TES)-Protected Prosapogenin by Selective Protection of Prosapogenin Hydroxyl Groups

1. In a 25-mL modified Schlenk flask, the solid mixture of prosapogenins 27 and 28 (˜0.55 g) is azeotroped from pyridine (5 mL), then additional pyridine (8 mL) is added, followed by TESOTf (2.0 mL, 8.8 mmol).

2. The reaction mixture is stirred for 2.75 days, then TESOTf (0.3 mL, 1.3 mmol) is added, followed by two further additions (0.1 mL each, 0.44 mmol each) 24 h and 48 h later, respectively (the last extra addition of TESOTf is situation-dependent and only required if the reaction is still incomplete after the first 4 days).

3. After a total of 5 days, the mixture is concentrated and passed through a short plug of silica gel eluted with hexanes/EtOAc (4:1 to 2:1). The eluate is concentrated, the resulting yellow oil is dissolved in MeOH/THF (1:1) (20 mL), and the solution is stirred for 3.5 days to remove the silyl esters by solvolysis.

4. The reaction mixture is concentrated and the resulting mixture of xylose- and rhamnose-containing (TES)₉-protected prosapogenin diacids is separated by silica gel chromatography (hexanes/EtOAc, 4:1 to 2:1) to afford purified xylose-containing protected prosapogenin (˜0.25 g, ˜22% yield) as a white solid.

Part C: Synthesis of Protected Quillaja Prosapogenin by Selective Esterification of Glucuronic Acid Carboxylic Acid in Protected Prosapogenin

1. In a 10-mL modified Schlenk flask, the prosapogenin diacid (81 mg, 41 μmol, 1.0 equiv.) is dissolved in DCM (0.7 mL) and pyridine (30 μL, 0.37 mmol, 9.0 equiv.) and TBP (102 mg, 0.41 mmol, 10 equiv.) are added, followed by benzyl chloroformate (15 μL, 0.11 mmol, 2.6 equiv.).

2. The reaction is stirred for 6 h, additional benzyl chloroformate (3.0 μL, 21 μmol, 0.51 equiv.) is added (the extra addition of CbzCl after the first 6 h depends on the progress of the reaction in each particular case; when purifying by silica gel chromatography, elution with benzene/EtOAc (100:0 to 24:1) can also be considered) and the reaction is stirred for another 20 h.

3. The mixture is concentrated and purified by silica gel chromatography (hexanes/EtOAc, 20:1 to 7:1) to afford selectively glucuronate-protected prosapogenin 30 (58 mg, 68%) as a white solid.

Acyl Chain Synthesis

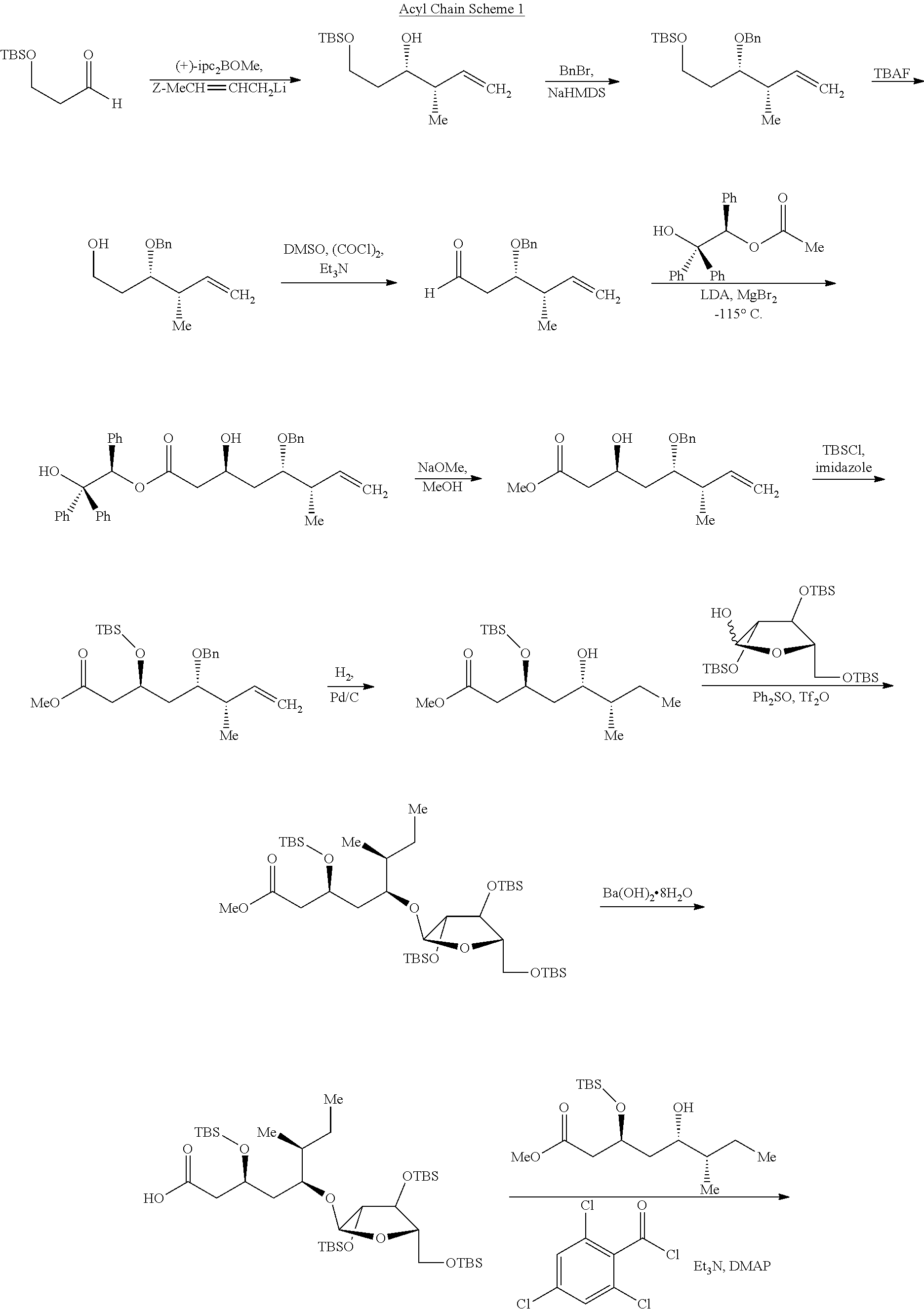

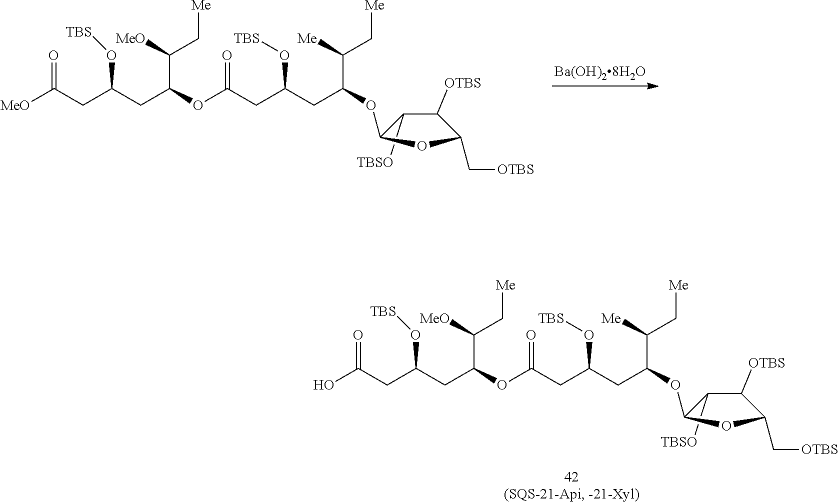

The product of Scheme 1 is assembled with an oligosaccharide produced as shown in the present application.

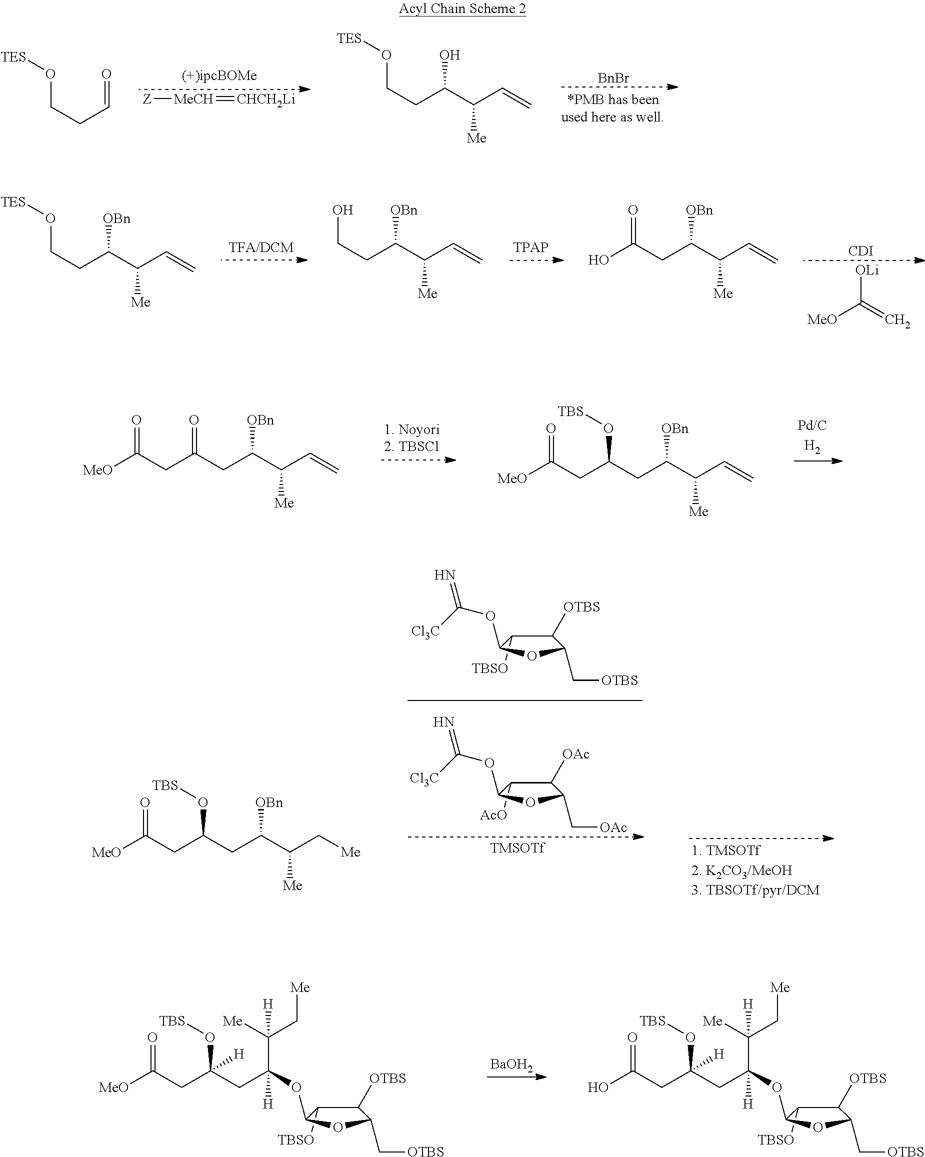

The product of Scheme 2 can be reacted as shown in Scheme 1 to produce the product shown in Scheme 1.

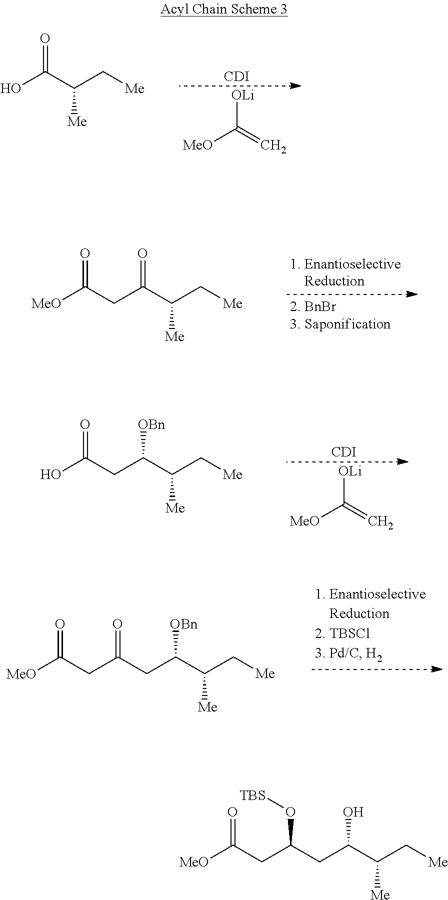

The product of Scheme 3 can be reacted as shown in Schemes 1 or 2 to produce the intermediate.

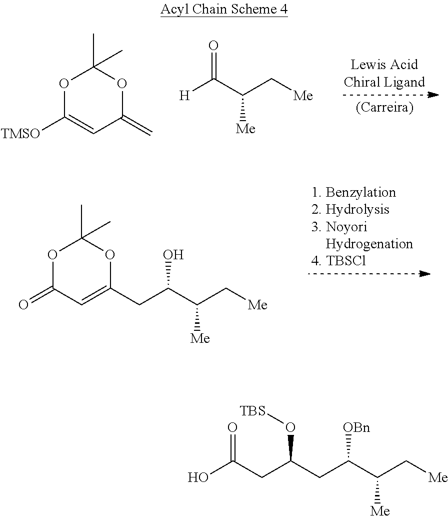

Protection/deprotection and enantioselective ketone reduction and sialylation gives the common intermediate. The product of Scheme 4 can be reacted as shown in Schemes 1 or 2 to produce the intermediate.

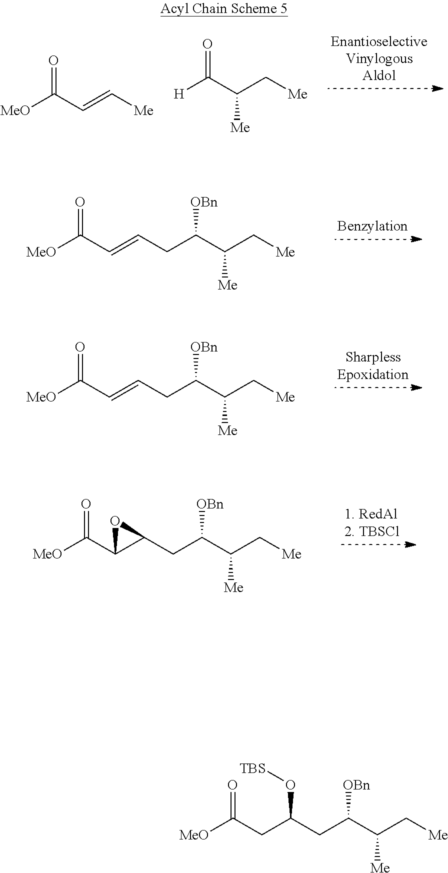

The product of Scheme 4 can be reacted as shown in Schemes 1 or 2 to produce the intermediate.

Oligosaccharide Synthesis

QS-21-Api:

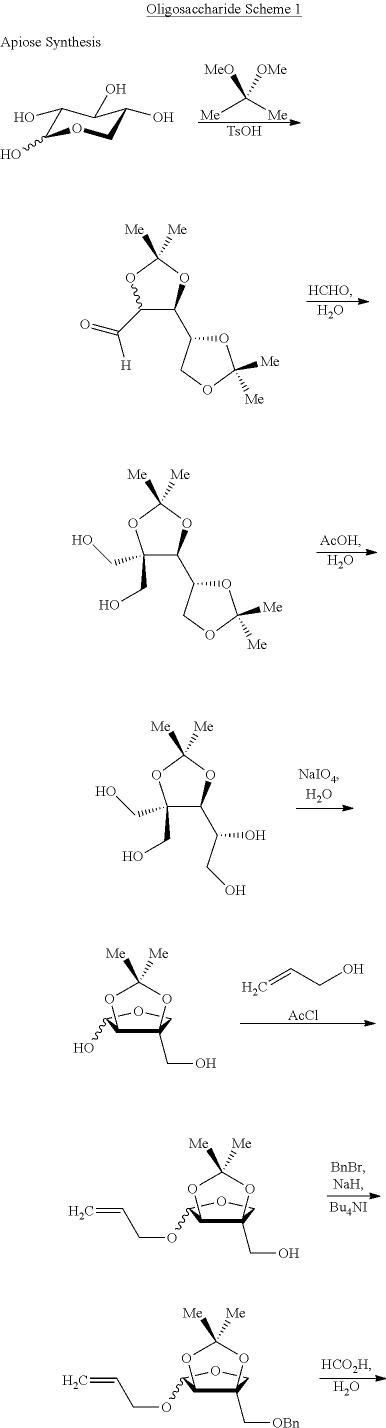

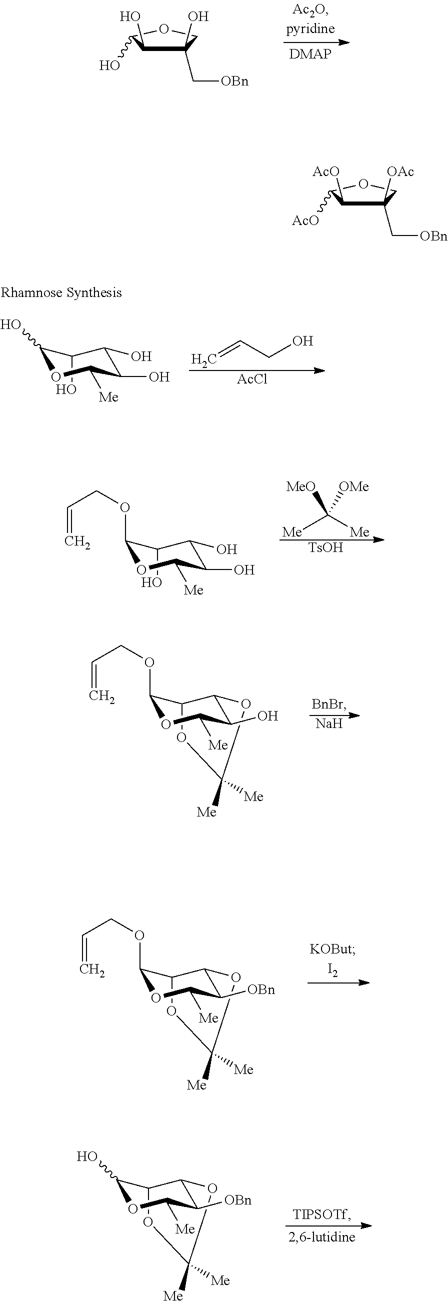

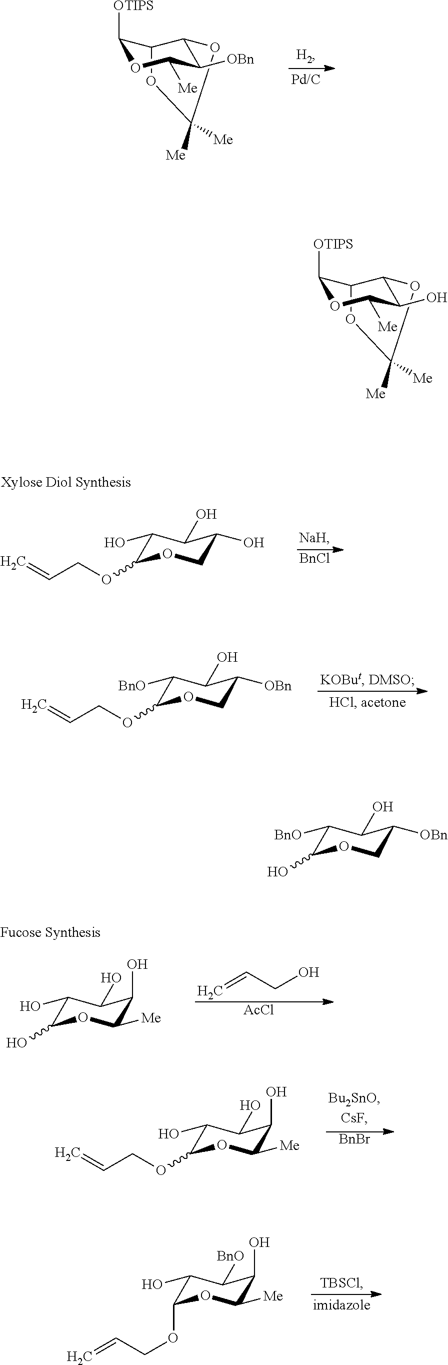

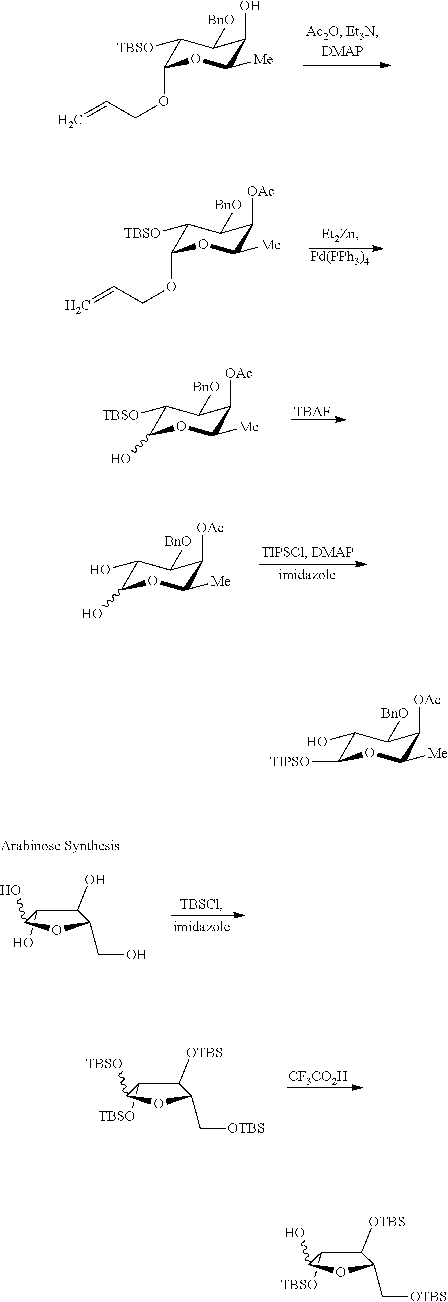

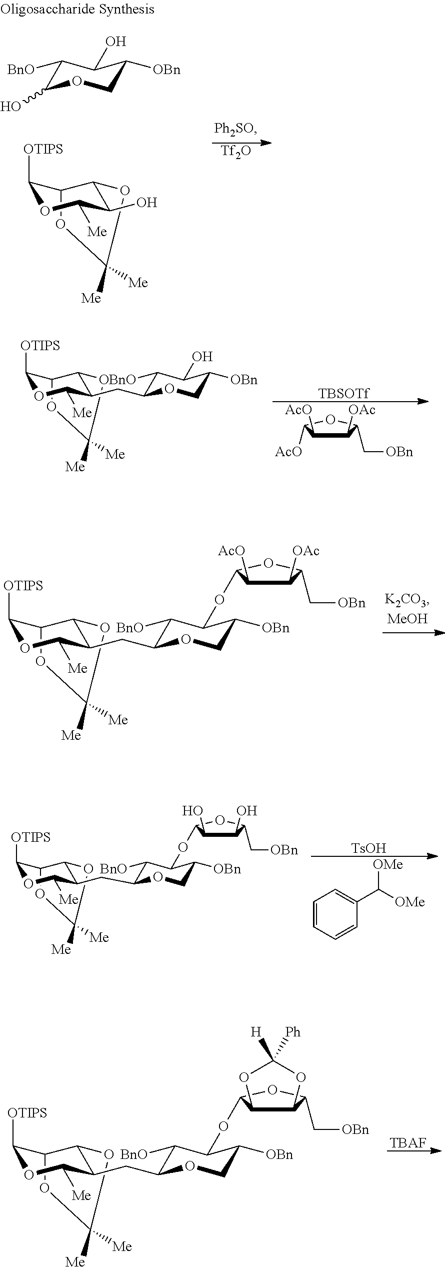

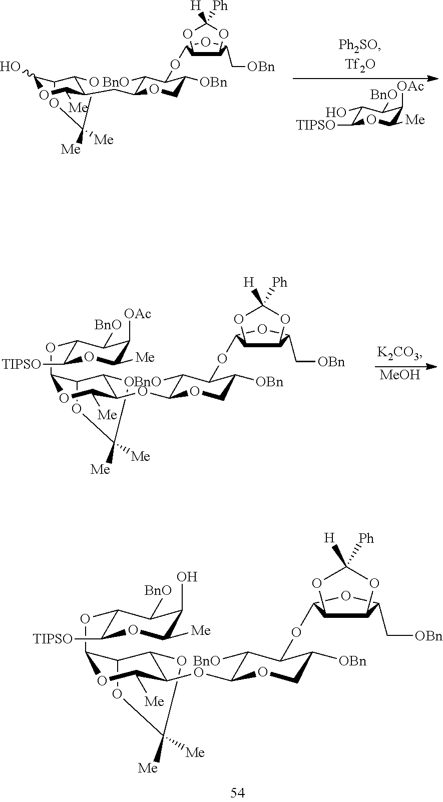

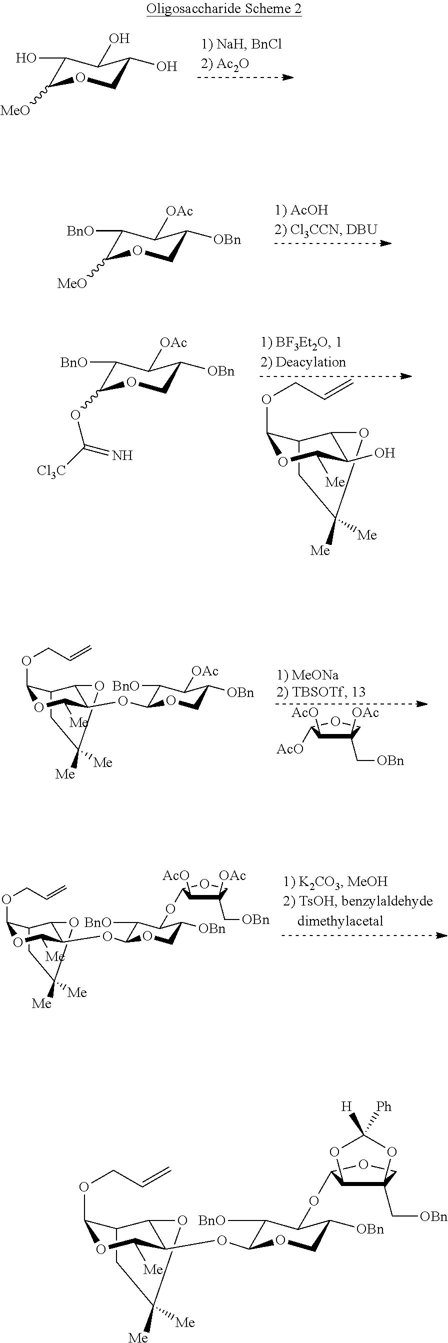

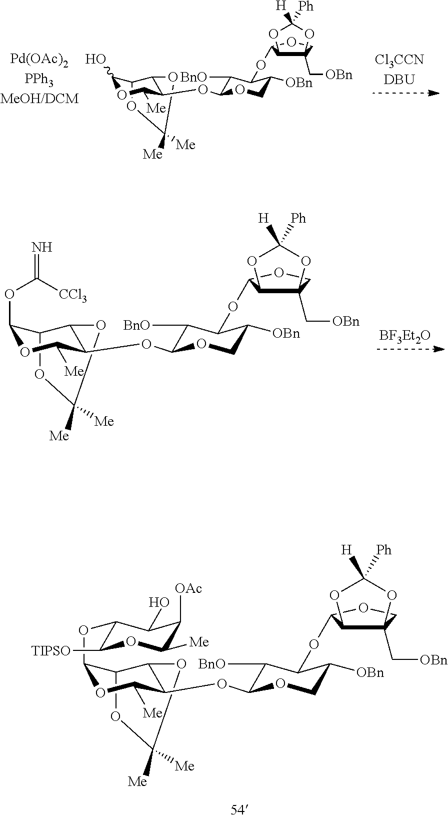

Synthesis of 54′ using common intermediate compound 1. Compound 54′ similar intermediate as compound 54.

QS-21 Xyl

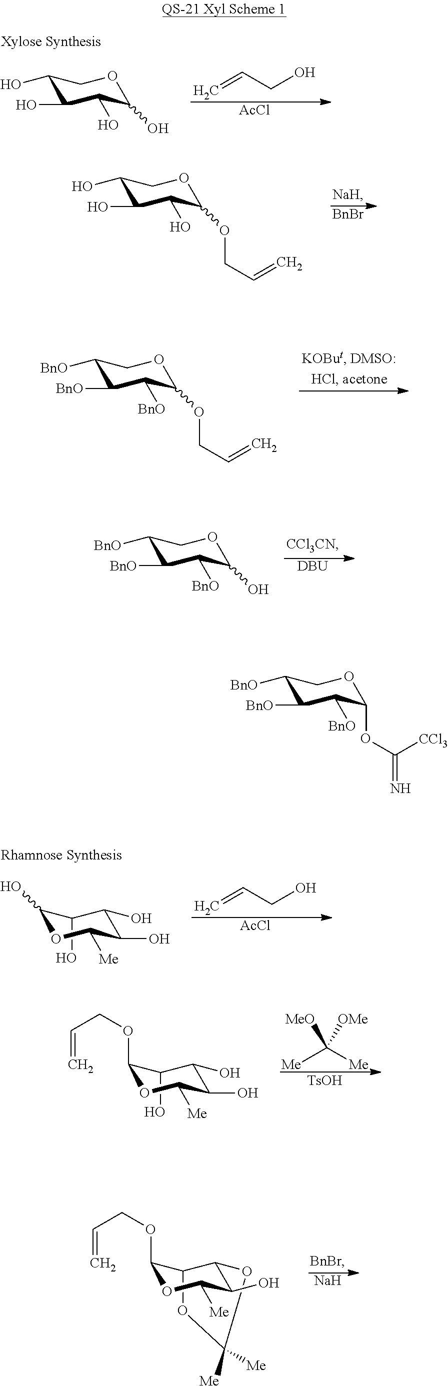

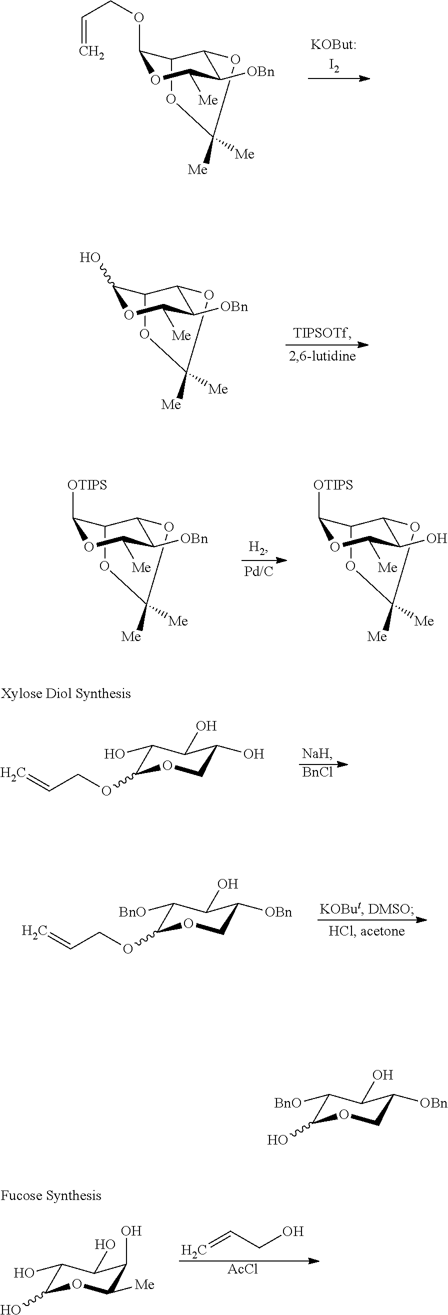

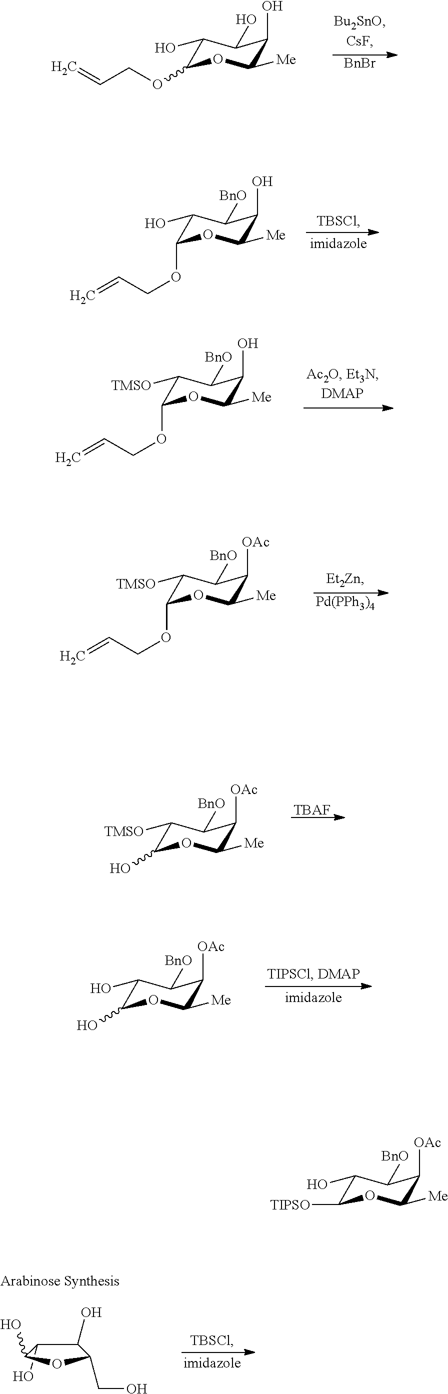

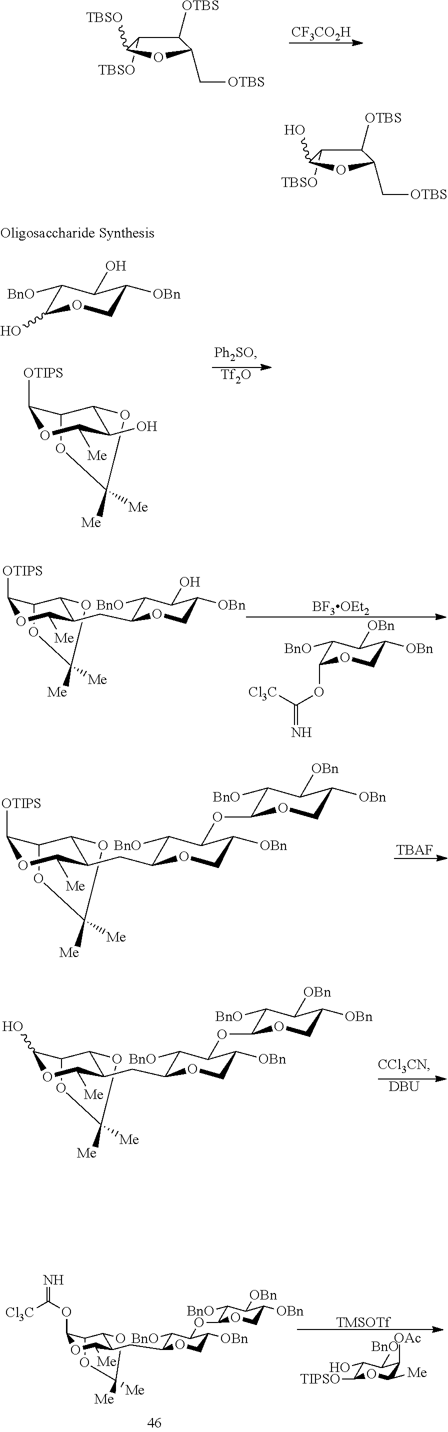

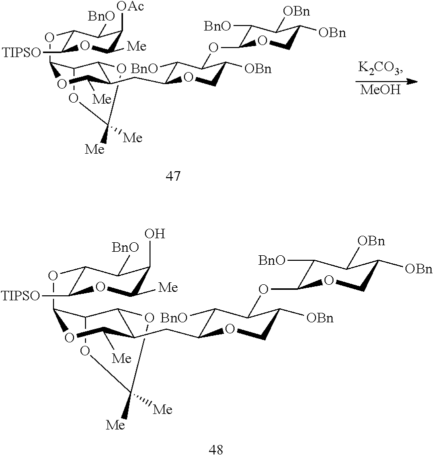

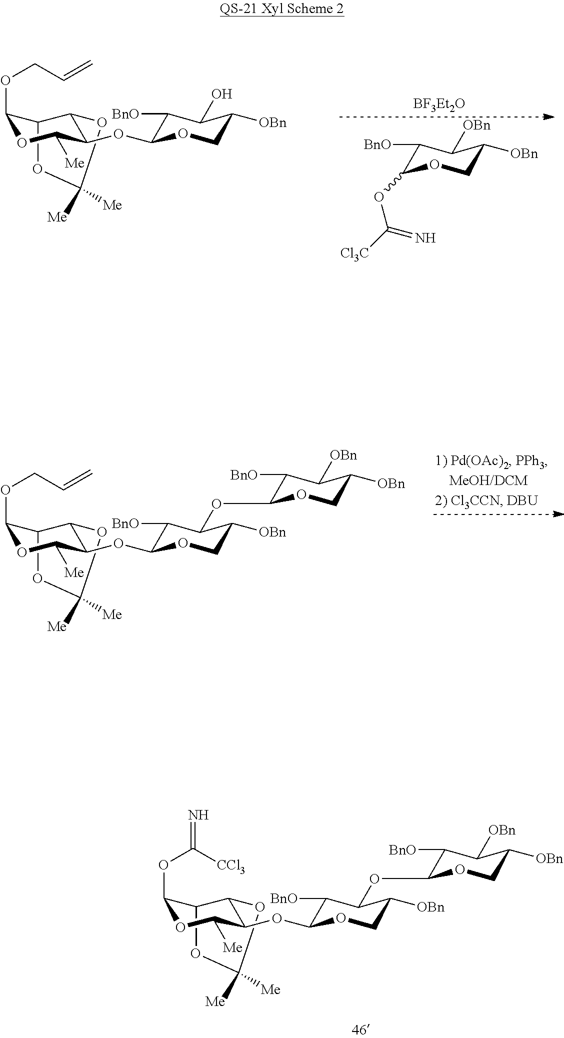

This scheme uses the common intermediate produced in the QS-21-Api synthesis shown previously. Compound 46 is similar to intermediate compound 46′.

The product of Scheme 2 can be reacted as shown in Scheme 1 to produce the intermediate.

Late-Stage Assembly

QS-21-Api

Assembly of acyl chain as shown previously with oligosaccharide shown previously. One of ordinary skill in the art would understand how to modify and/or use compound 54′ in the scheme shown herein such that compounds 54 and 54′, or modified versions thereof, are interchangeable.

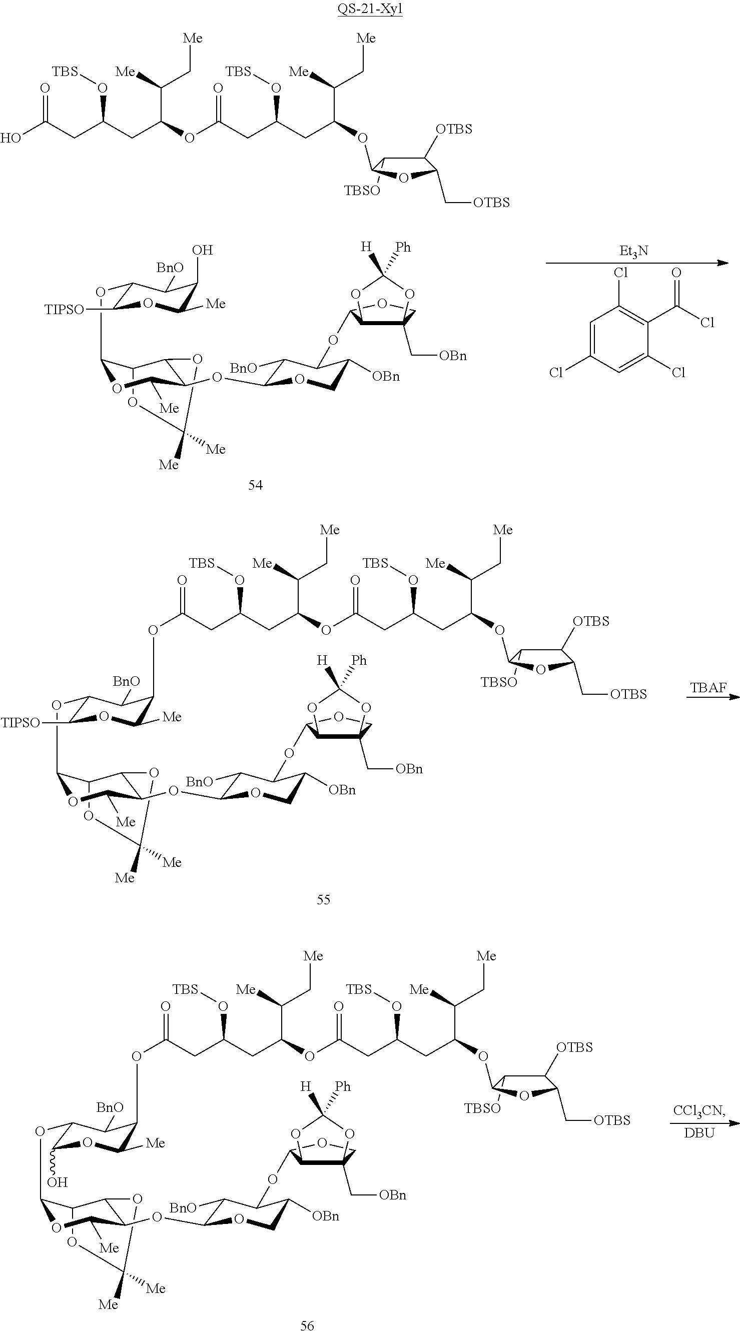

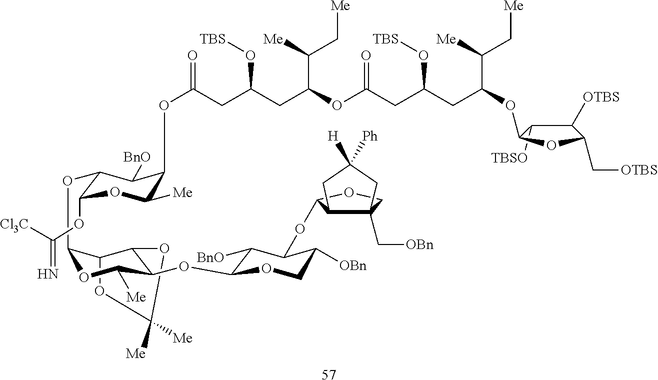

Assembly of acyl chain as shown previously with oligosaccharide shown previously.

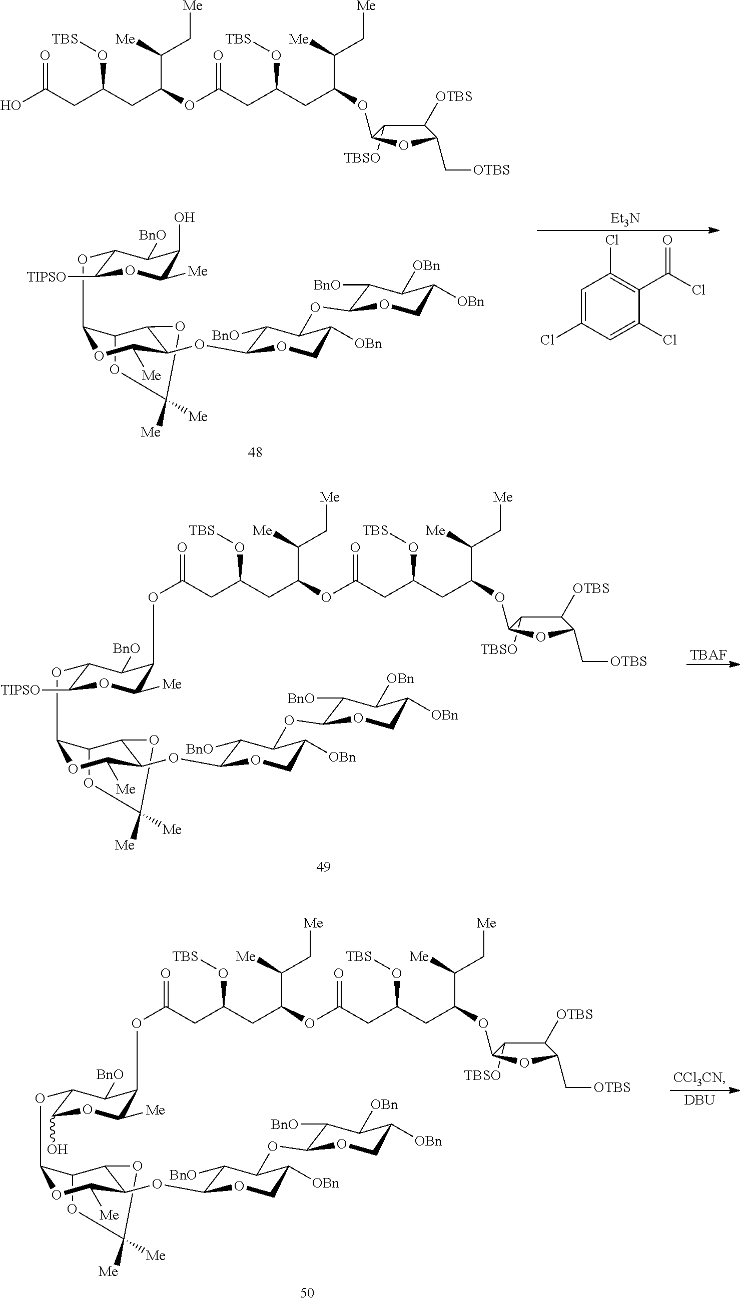

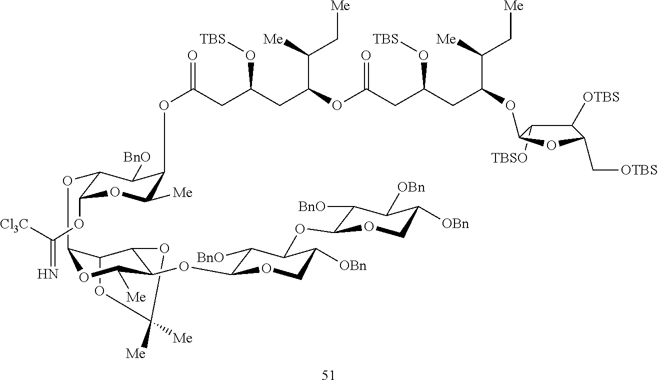

Coupling and Deprotection

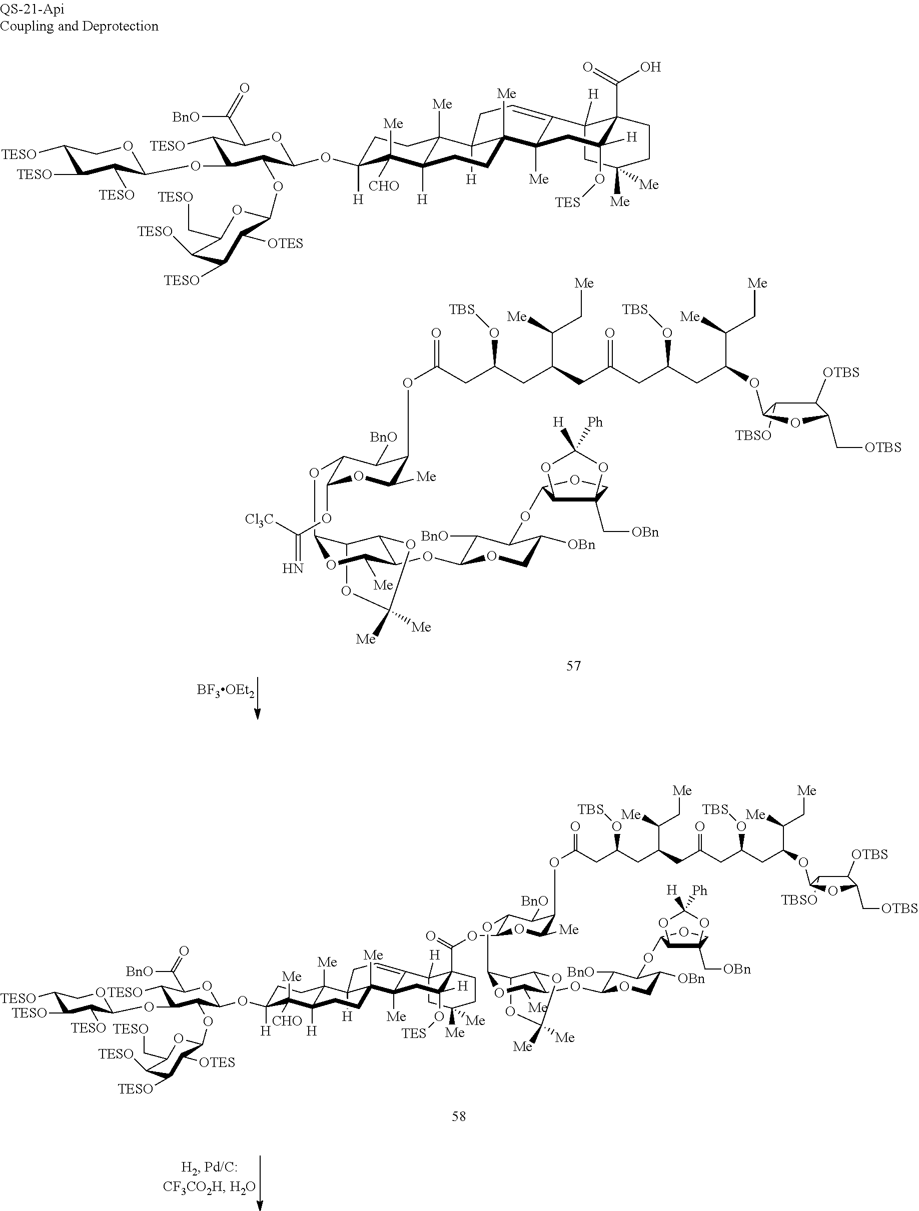

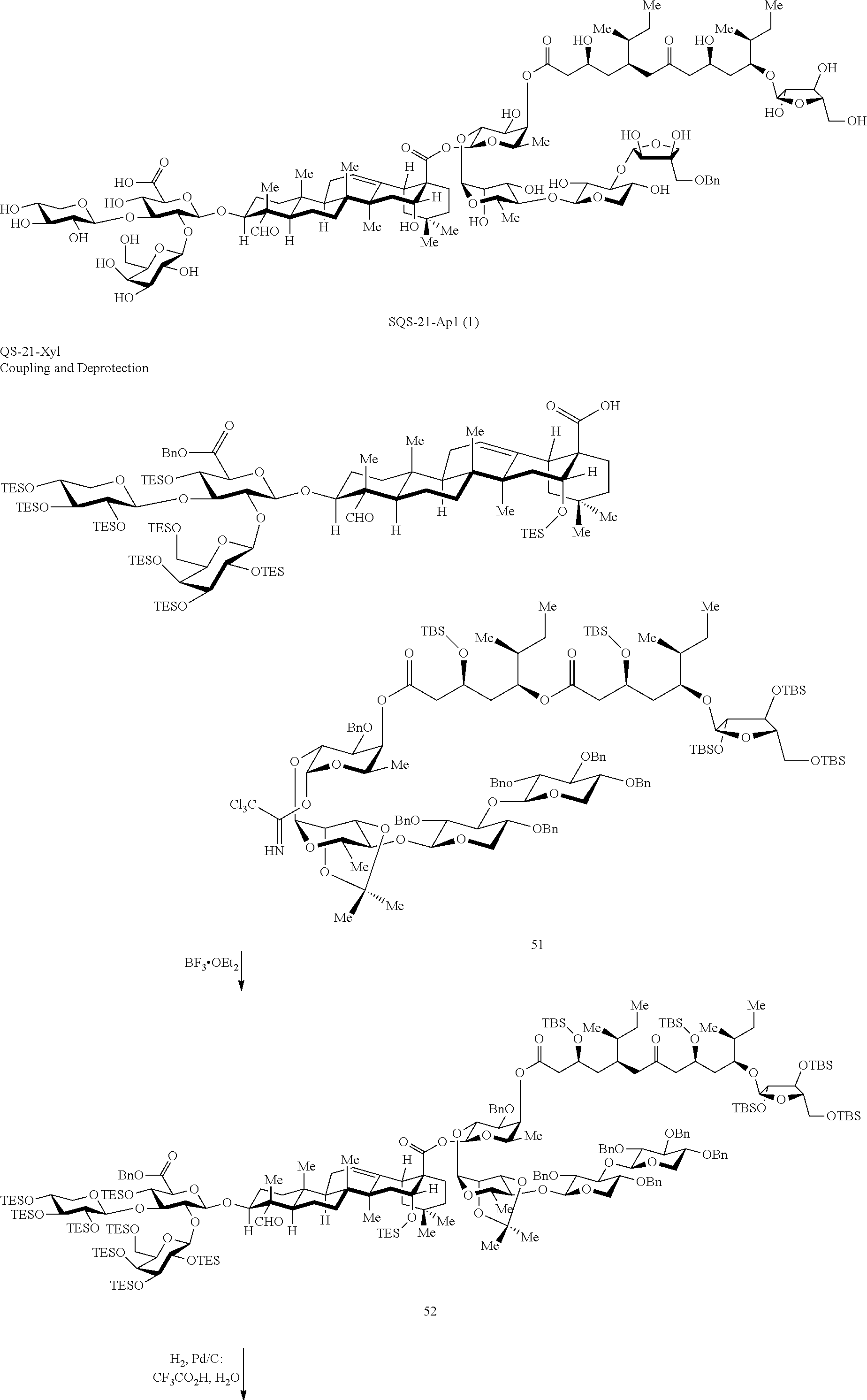

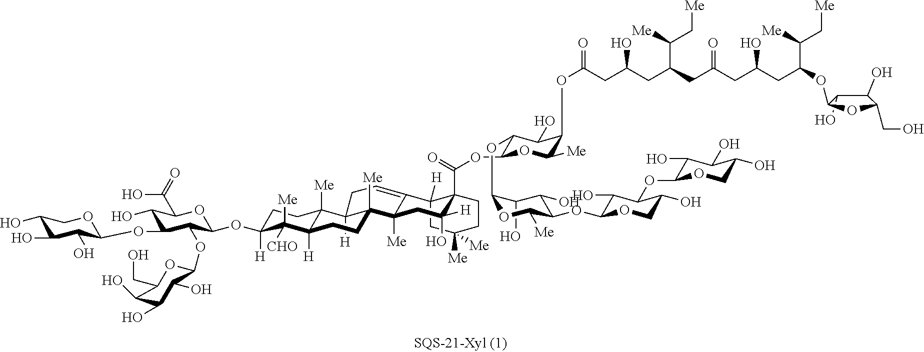

### Example 3: Prevnar-13-CRM197 Conjugate Vaccine Adjuvanted with Synthetic Saponins

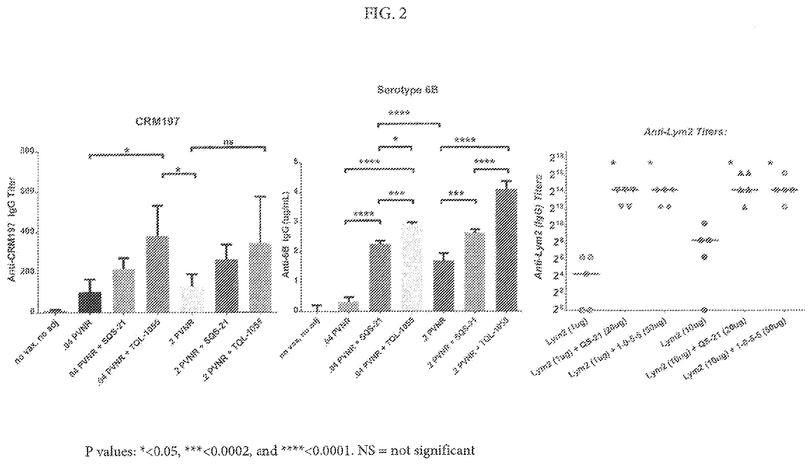

The impact of synthetic QS-21 and TQL-1055 (Compound 26) on antibody titers induced by the FDA approved human pmeumococcal-CRM197 conjugate vaccine, Prevnar-13, was tested. Mice were immunized with Prevnar-13 in the presence or absence of synthetic saponin adjuvants at two different Prevnar dose levels (0.04 mcg and 0.2 mcg). Mice were immunized once at Day 0 and bled on Day 21 for serum analysis. FIG. 2 of the present application reports data obtained in this study, showing the immunogenicity of high or low dose Prevnar-13 or of Lym2-CRM197 conjugate in combination with synthetic QS-21 (SQS-21) or TQL-1055 (Compound 26).

### Example 4: Impact of TQL-1055 (Compound 26) and QS-21 on Tdap Vaccine Adacel Immunogenicity

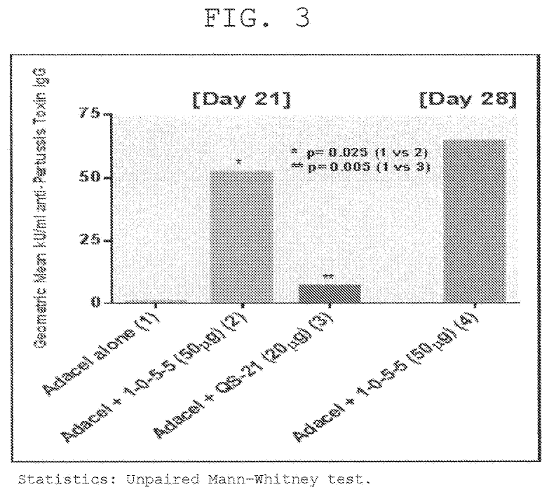

Adacel doses containing 1, 0.3, and 0.1 mcg of pertussis toxin per mouse were administered subcutaneously (SC, with no immunological adjuvant), using 2 vaccinations 4 weeks apart, resulting in a mean of 1,618 mcg, 898 mcg, and 107 mcg respectively of anti-PT antibody per ml of serum drawn 2 weeks after the second vaccination. The 0.1 mcg dose was indistinguishable from unvaccinated controls (96 mcg/ml). A 0.5 mcg dose of Adacel was selected for a pharmacology/toxicology (pharm/tox) study. The serological results for this study are summarized in FIG. 3 of the present application. Antibody levels in the groups of 5 mice 2 weeks after the second SC immunization were augmented by 70 fold (726 to 52,344) with TiterQuil-1055 (TQL-1055/Compound 26) (and further increased 2 weeks later) and 10 fold with QS-21 compared to immunization with Adacel alone. No weight loss was detected in the mice receiving 50 mcg of TiterQuil-1055 while the 20 mcg QS-21 injected mice lost 8-9% of their body weight.

### Example 5: Impact of TiterQuil-1-0-5-5 and QS-21 on Hepatitis B Vaccine Engerix-B Immunogenicity

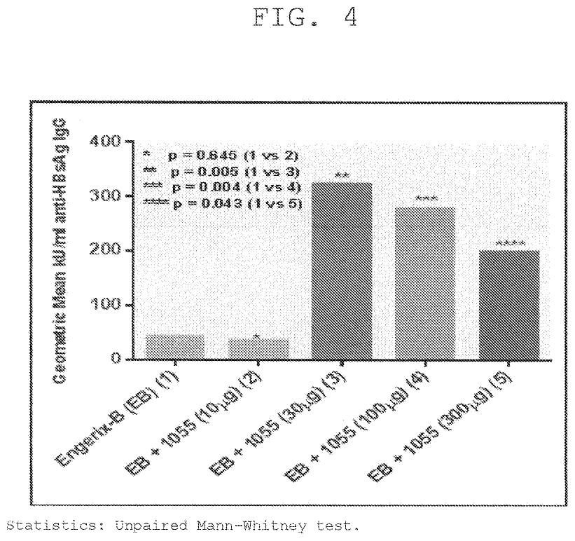

Experiments were conducted with Engerix-B (HBV adult vaccine) in groups of 10 mice. Initially 3 mcg, 1 mcg, 0.3 mcg, 0.1 mcg, and 0.03 mcg Engerix-B doses per mouse were tested. Mean resulting anti-HBsAg antibody levels were 92,512 mcg/ml, 64,255 mcg/ml, 24,847 mcg/ml, 3,682 mcg/ml, and 910 mcg/ml respectively, with the 0.03 dose being indistinguishable from controls (821 mcg/ml). The 0.3 mcg dose of Engerix-B was selected for further studies and this dose was used mixed with various doses of TiterQuil-1055 (TQL-1055/Compound 1-4). The resulting geometric mean antibody concentrations are summarized in FIG. 4 of the present application. While 10 mcg of TiterQuil-1055 appeared to have no serologic effect, mixture of 30 and 100 mcg TiterQuil-1055 with Engerix-B resulted in a >6 and 5-fold increase (respectively) in antibody levels compared to Engerix-B alone. Lack of antibody increase or decreasing responses at TiterQuil-1055 doses above 50 mcg per mouse has been a consistent finding. No weight loss was seen at the 30 mcg TiterQuil-1055 dose and only 4% and 5% at the 100 and 300 mcg doses.

### Example 6: Results of a Pilot Pharmacology/Toxicology with Adacel QS-21 and TiterQuil-1055

A pharm/tox study was conducted in 7 groups of 5 mice: 1) PBS alone, 2) 50 mcg TiterQuil-1055, 3) 20 mcg QS-21, 4) Adacel 2.5 mcg pertussis toxin (⅕ the human dose), 5) Adacel+QS-21 (20 mcg QS-21), 6) Adacel+TiterQuil-1055 (50 mcg), 7) Adacel+TiterQuil-1055 (50 mcg). Mice were vaccinated SC on days 1 and 15, weighed daily, and bled and sacrificed on day 22, except for group 7 which was sacrificed on day 29. No changes in blood chemistry or hematology results were seen in any group. 7-9% weight loss was seen in all mice in groups 3 and 5 (in agreement with prior results of QS-21) and in no other mice. Histopathology of 33 different tissues was performed on all mice. Detected abnormalities were restricted to the liver. Moderate to severe hepatocellular cytoplasmic vacuolization was seen in all mice in groups 4-6 (completely attributable to the pertussis vaccine at this dose, groups 5 and 6 were no more severe than group 4) but no mice in groups 1 or 2. This abnormality was short lived and was no detected in group 7, which was sacrificed one week after groups 1-6. Mild vacuolar changes were seen in all mice in group 3 (QS-21 alone). No changes at all were seen in groups 1 and 2 (PBS and TiterQuil-1055).

### Example 7: Stability and Hemolytic Activity of Compound 1-4 (TQL-1055/TiterQuil-1-0-5-5)

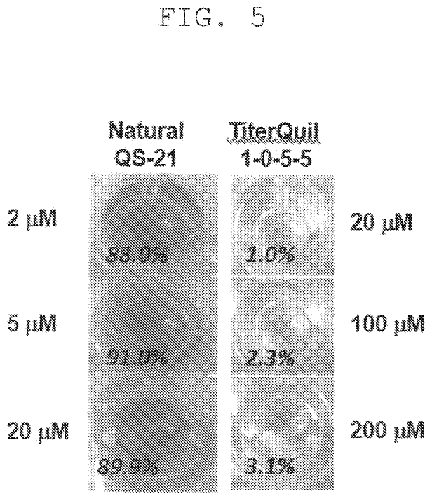

Natural and synthetic QS-21 (SQS-21 or SAPONEX®) and a variety of analogs were tested for hemolytic activity. This data clearly demonstrates that QS-21 is highly hemolytically active whereas several of t0he structural analogs, particularly Compound 1-4 (TiterQuil-1-0-5-5/TQL-1055), demonstrated much lower or undetectable hemolytic activity in addition to increased stability. FIG. 5 depicts results a hemolytic assay performed with TiterQuil-1055. In a companion toxicity study three days after immunization, animals that received 20 mcg of QS-21 have lost 8-10% of their body mass on average, whereas PBS, TiterQuil-101 and TiterQuil-1055 recipients have gained 5% on average (normal weight gain in young mice). Without being bound by theory, hemolytic activity may be a direct result of degradation of QS-21 under physiologic conditions and TiterQuil-1055's lack of hemolytic activity may result from improved stability. After two weeks at 37° C., 20% of QS-21 degraded, whereas TiterQuil-1055 was still intact without detectable degradation.

1. A method of synthesizing a compound according to Formula I or an intermediate thereof, comprising at least one of the following steps (a)-(g):
a. purifying semi-purified Quillaja Bark extract as depicted, 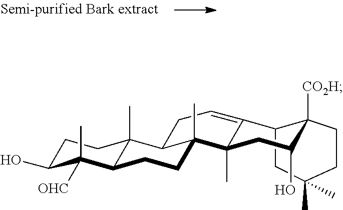
b. protecting a hydroxyl with triethylsilyl groups, 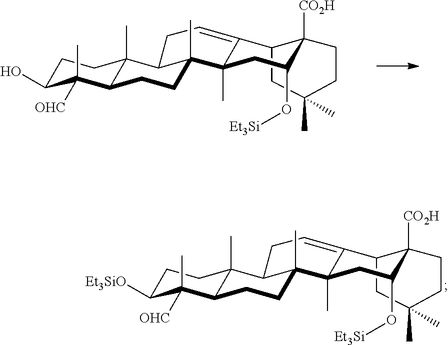
c. reacting a triethylsilyl protected compound with C-1, 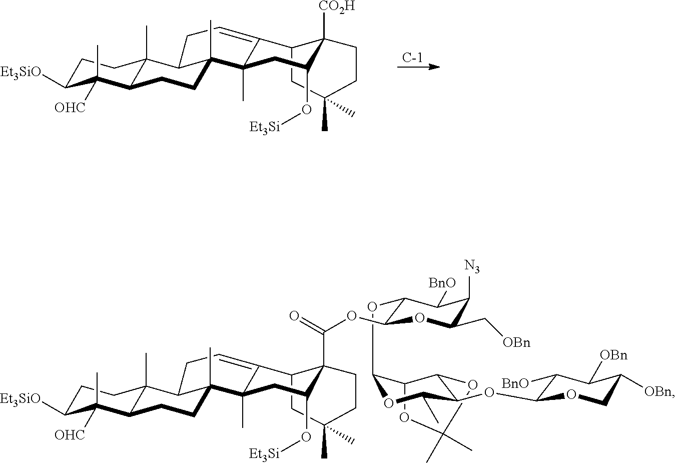
wherein C-1 is 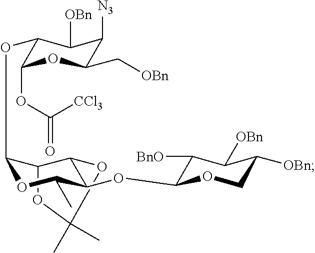
d. reducing N³ to NH₂, 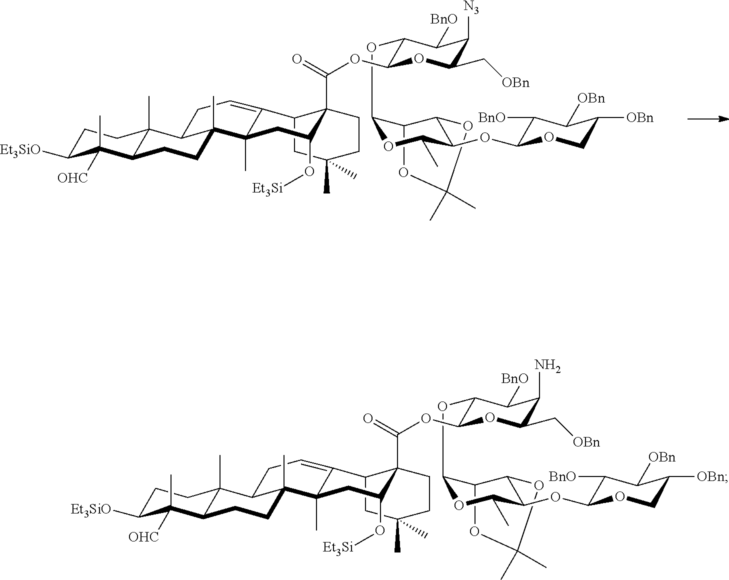
e. reacting amine moiety carboxylic acid to from amide linkage 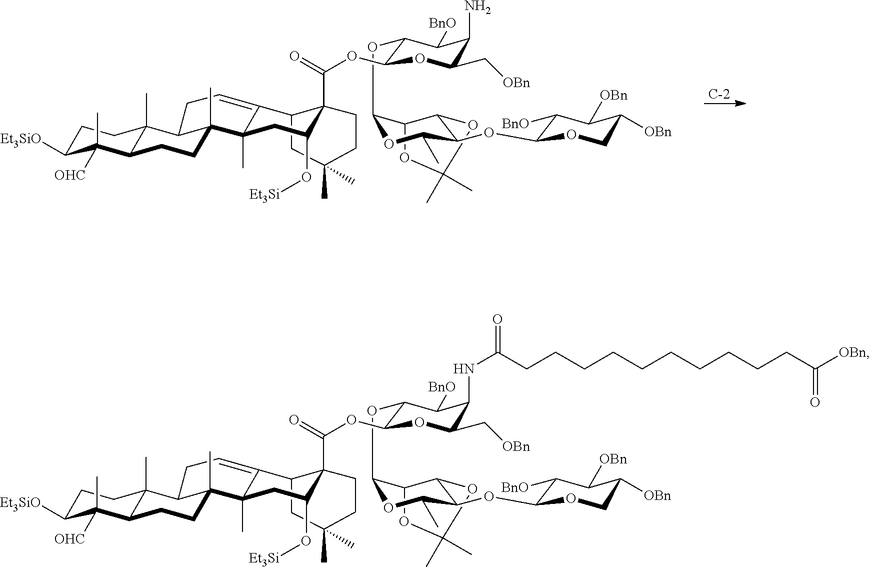
wherein C-2 is OH—C(O)—(CH₂)₁₀—C(O)—OBn;
f. deprotecting by hydrogenation 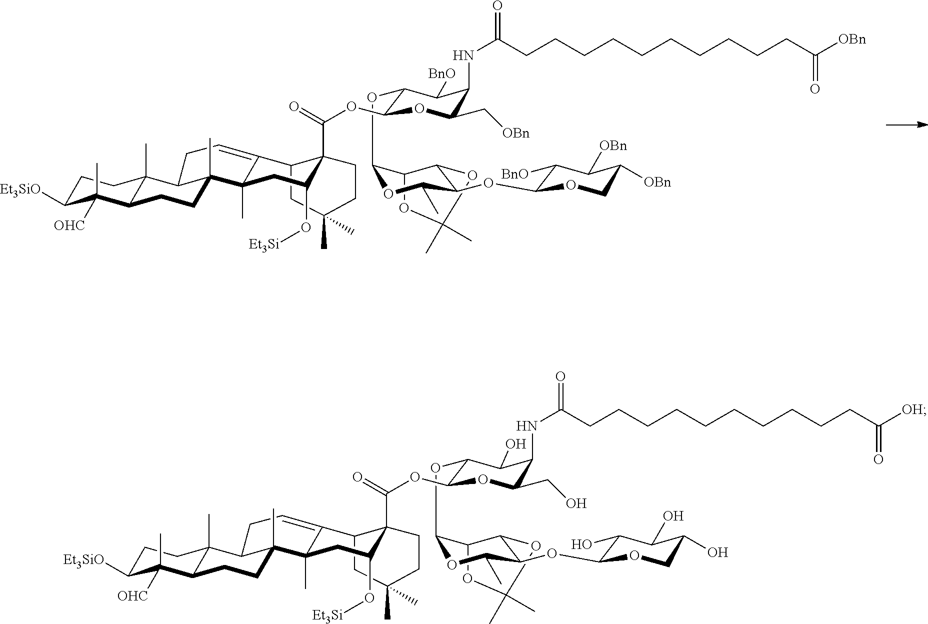
g. deprotecting with trifluoroacetic acid and isolating a compound:

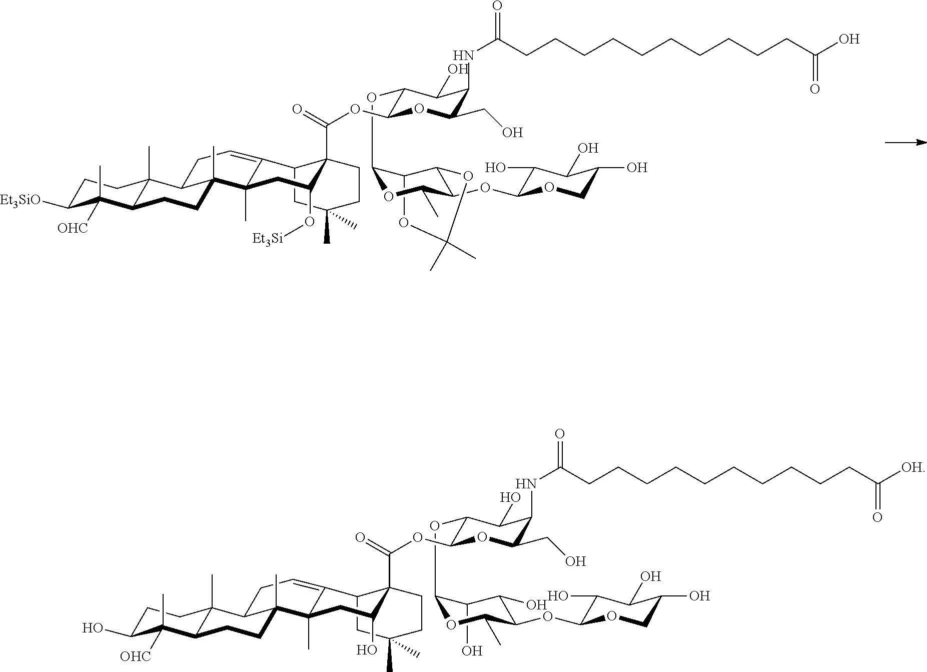

2. The method according to claim 1, wherein the compound of Formula I is:

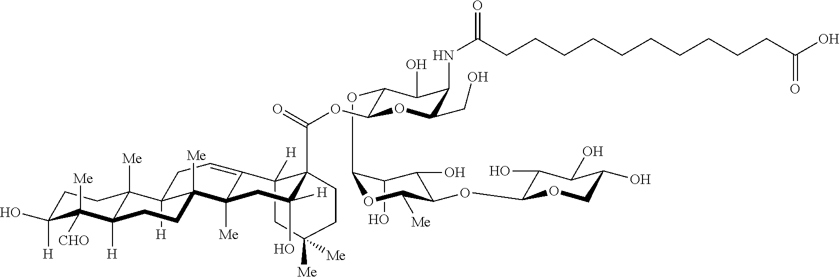

3. A pharmaceutical composition, comprising:
the compound obtained by the process according to claim 2 and
an immunologically effective amount of an antigen associated with a bacteria or virus causing a disease selected from the group consisting of Hepatitis B, pneumococcus, diphtheria, tetanus, pertussis, or Lyme disease including the closely related spirochetes of the genus Borrelia such as, B. burgdorferi, B. garinii, B. afzelli, and B. japonica.

4. A pharmaceutical composition according to claim 3, wherein the immunologically effective amount of an antigen is associated with Hepatitis B virus.

5. A pharmaceutical composition according to claim 3, wherein the immunologically effective amount of an antigen is associated with pneumococcus bacterium.

6. A pharmaceutical composition according to claim 3, wherein the immunologically effective amount of an antigen is associated with Corynebacterium diphtheria bacterium.

7. A pharmaceutical composition according to claim 3, wherein the immunologically effective amount of an antigen is associated with Clostridium tetani bacterium.

8. A pharmaceutical composition according to claim 3, wherein the immunologically effective amount of an antigen is associated with Bordetella pertussis bacterium.

9. A pharmaceutical composition according to claim 3, wherein the immunologically effective amount of an antigen is associated with a bacterium causing Lyme disease or a spirochete of the genus Borrelia selected from the group consisting of B. burgdorferi, B. garinii, B. afzelli, and B. japonica.

10. A method of synthesizing a compound of Formula II, or an intermediate thereof, comprising a reaction step selected from at least one of the following steps:

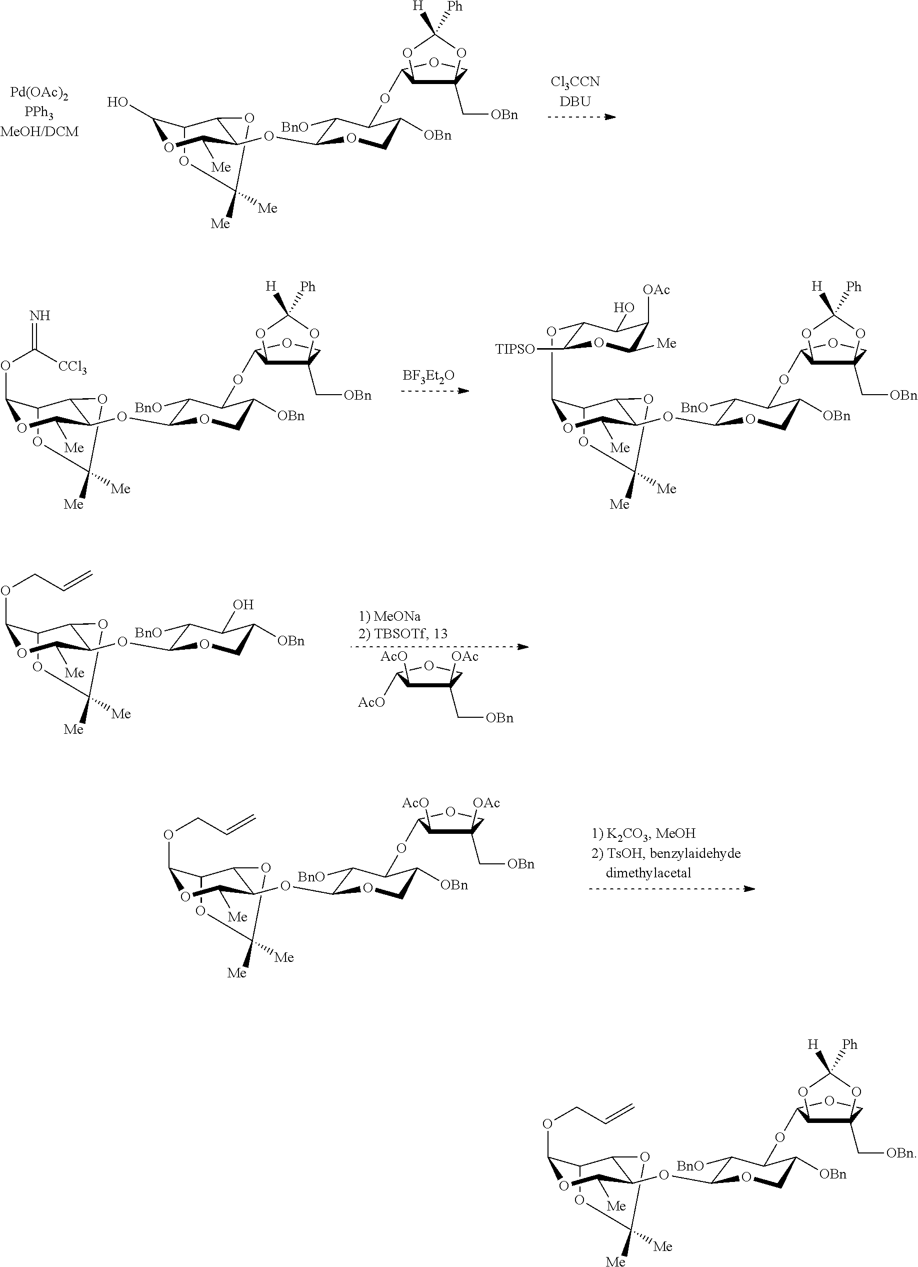

11. The method according to claim 10, wherein the compound of Formula II is II SQS-21-Api.

12. A method of synthesizing a compound of Formula II, or an intermediate thereof, comprising a reaction step selected from at least one of the following steps:

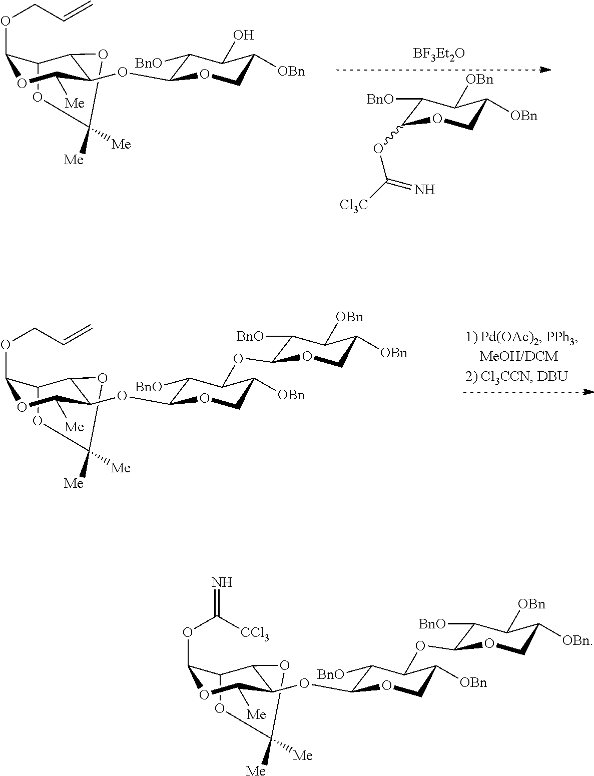

13. The method according to claim 12, wherein the compound of Formula II is SQS-21-Xyl.

14. A method of synthesizing a compound of Formula II or an intermediate thereof, comprising a reaction step selected from at least one of the following steps:

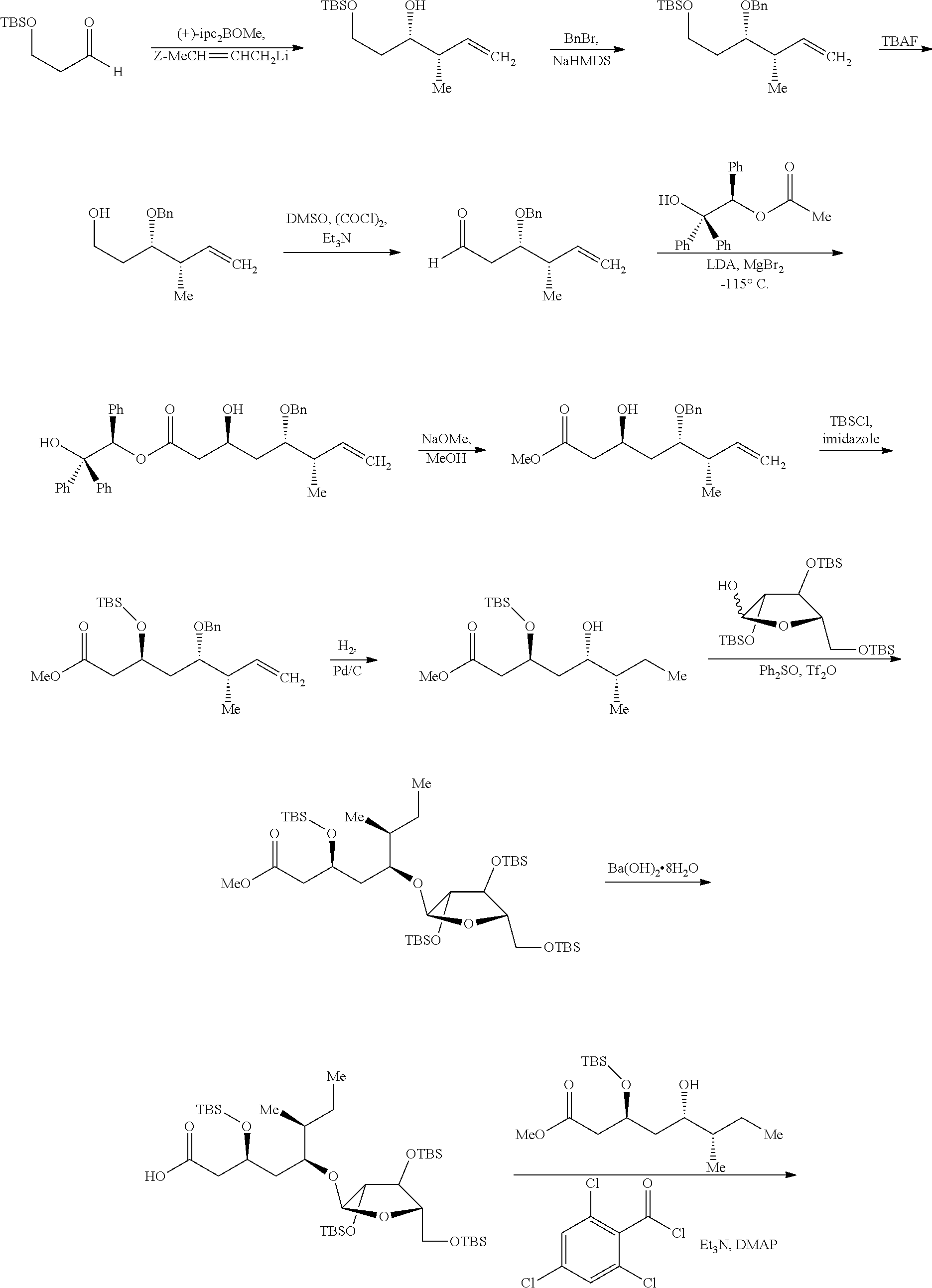

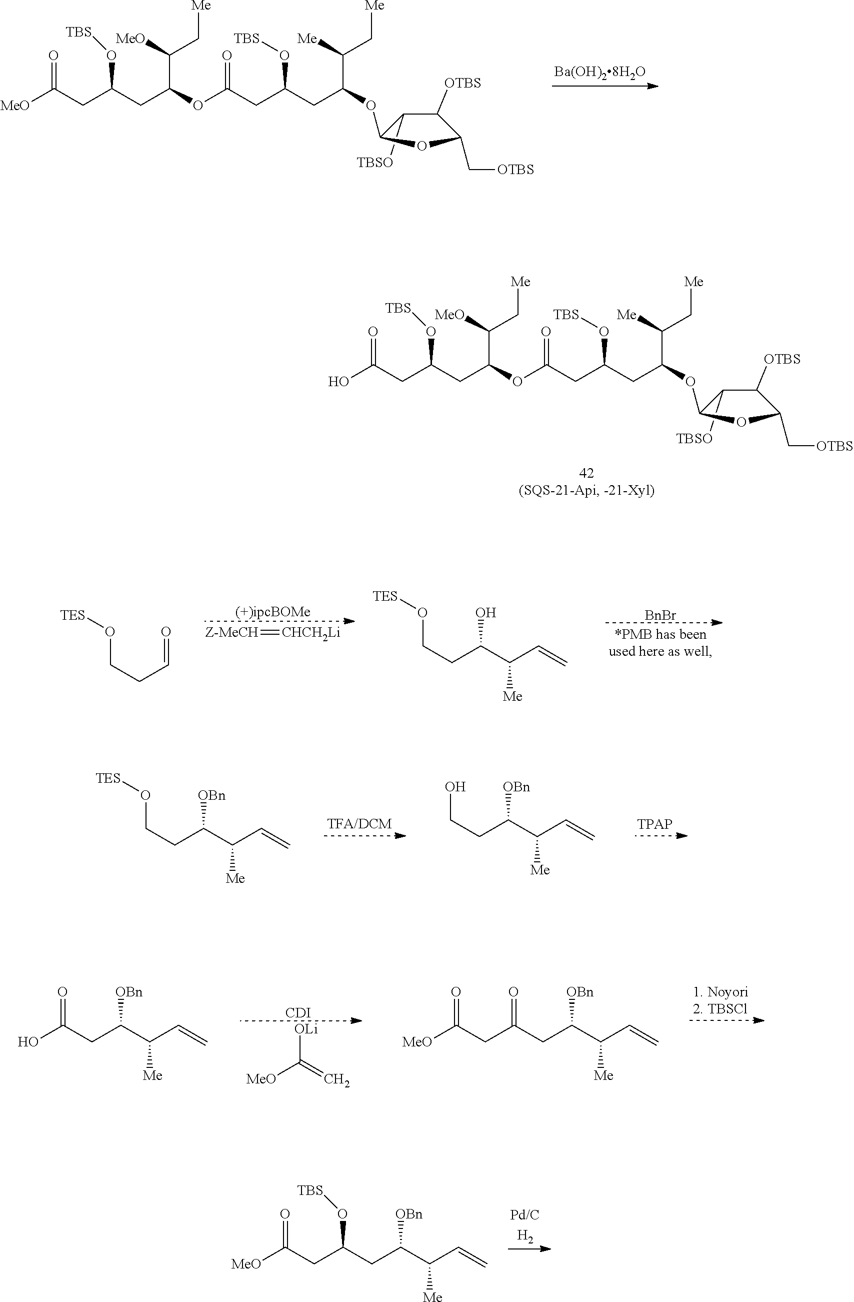

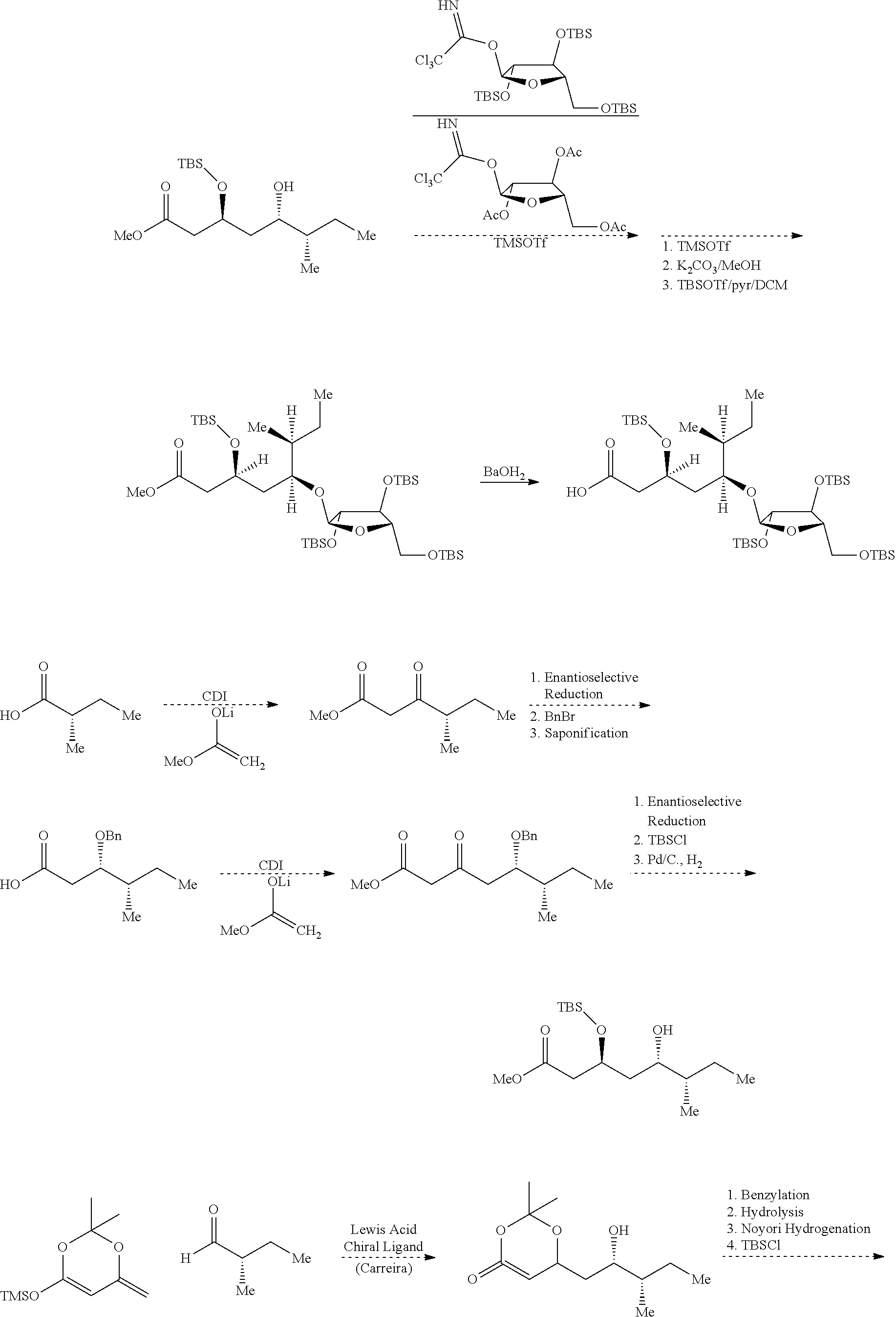

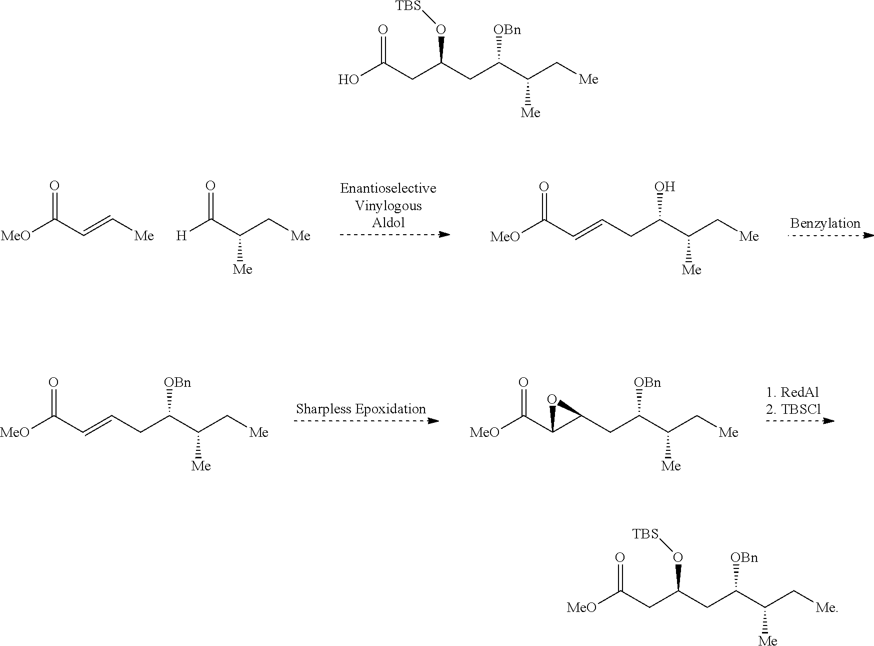

15. The method according to claim 14, wherein the compound of Formula II is SQS-21-Xyl or SQS-21-Api.

16. A pharmaceutical composition, comprising:
the compound obtained by the process according to claim 10, and
an immunologically effective amount of an antigen associated with a bacteria or virus causing a disease selected from the group consisting of Hepatitis B, pneumococcus, diphtheria, tetanus, pertussis, or Lyme disease including the closely related spirochetes of the genus Borrelia such as, B. burgdorferi, B. garinii, B. afzelli, and B. japonica.

17. A pharmaceutical composition according to claim 16, wherein the immunologically effective amount of an antigen is associated with Hepatitis B virus.

18. A pharmaceutical composition according to claim 16, wherein the immunologically effective amount of an antigen is associated with pneumococcus bacterium.

19. A pharmaceutical composition according to claim 16, wherein the immunologically effective amount of an antigen is associated with Corynebacterium diphtheria bacterium.

20. A pharmaceutical composition according to claim 16, wherein the immunologically effective amount of an antigen is associated with Clostridium tetani bacterium.

21. A pharmaceutical composition according to claim 16, wherein the immunologically effective amount of an antigen is associated with Bordetella pertussis bacterium.

22. A pharmaceutical composition according to claim 16, wherein the immunologically effective amount of an antigen is associated with a bacterium causing Lyme disease or a spirochete of the genus Borrelia selected from the group consisting of B. burgdorferi, B. garinii, B. afzelli, and B. japonica.

23. A process of isolating a compound 19: 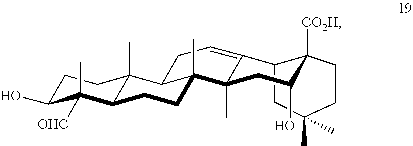
said process comprising extracting and purifying the compound 19 from Soapwort seed extract.

24. A process of isolating a mixture of Major Quillaja Prosapogenin and Minor Qillaja Prosapogenin: 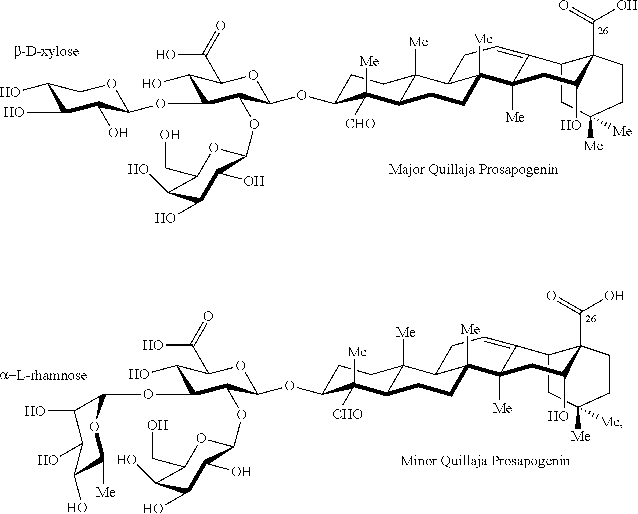
said process comprising extracting and purifying the mixture of Major Quillaja Prosapogenin and Minor Qillaja Prosapogenin from Sortwort seed extract.
